(19) United States
(12) Patent Application Publication
(10) Pub. No.: US 2024/0024289 A1
(43) Pub. Date: Jan. 25, 2024

(54) TREATMENT OF BIPOLAR DISORDERS AND PSYCHOSIS USING DEXMEDETOMIDINE HYDROCHLORIDE

(71) Applicant: BioXcel Therapeutics, Inc., New Haven, CT (US)

(72) Inventors: Frank YOCCA, New Haven, CT (US); Robert RISINGER, New Haven, CT (US); Michael DE VIVO, New Haven, CT (US); Friso POSTMA, New Haven, CT (US)

(21) Appl. No.: 18/030,405

(22) PCT Filed: Oct. 8, 2021
(86) PCT No.: PCT/US2021/054171

§ 371 Date: Apr. 5, 2023

Related U.S. Application Data
(60) Provisional application No. 63/089,135, filed on Oct. 8, 2020.

(51) Int. Cl. A61K 31/4174; A61P 25/18; A61K 9/00

(52) U.S. Cl. CPC A61K 31/4174; A61P 25/18; A61K 9/006

(57) ABSTRACT

Disclosed herein are methods and composition for treating bipolar disorders and psychosis by administering dexmedetomidine. The methods and composition alleviate mania, hypomania, psychosis, and depression in the subjects, providing improved therapeutic outcomes.

### CROSS-REFERENCE TO RELATED APPLICATIONS

This application claims priority to U.S. Provisional Application Ser. No. 63/089,135, filed Oct. 8, 2020, the contents of which is hereby incorporated by reference in its entirety for all purposes.

### FIELD

Disclosed herein are methods of treating a human subject having mania, hypomania, mixed mania, dysphoric mania, or depression associated with a bipolar illness such as bipolar disorder (e.g. bipolar I disorder and bipolar II disorder).

The disclosure also relates to methods of treating a human subject having psychosis associated with schizophrenia, schizoaffective disorder, depression, dementia and a bipolar illness such as bipolar disorder (e.g. bipolar I disorder and bipolar II disorder).

### BACKGROUND

Bipolar disorder, also known as manic depression, manic depressive disorder or bipolar affective disorder, is a psychiatric diagnosis that describes a category of mood disorders defined by the presence of one or more episodes of abnormally elevated mood clinically referred to as mania or, if milder, hypomania. Individuals who experience manic episodes also commonly experience depressive episodes or symptoms, or mixed episodes in which features of both mania and depression are present at the same time. The presence of both symptoms of depression and mania simultaneously as well as simply depressive symptoms with irritability is often termed dysphoria or dysphoric mood. A dysphoric mood state is associated with poor outcomes and risk of suicide. Typically dysphoric mood state is associated with a major depressive episode (depression) or bipolar disorder with mixed mood state (symptoms of a major depressive episode with symptoms of mania that do not rise to level that meet criterion for diagnosis of mania). Severe mood disorders, can be associated with psychotic symptoms. Major depressive disorder as well as bipolar disorders such as extreme manic episodes can impair one's normal judgement and sometimes lead to psychotic symptoms such as delusions and hallucinations.

Manic episodes typically emerge over a period of days to weeks, but onset within hours is possible, usually in the early morning hours. An untreated episode of either depression or mania can be as short as several weeks or last as long as 8 to 12 months. In rare cases, patients have an unremitting persistent course. Acute manic and mixed episodes are frequently associated with severe behavioral, physical, functional, and cognitive disturbances, all of which can have important personal and social consequences. For the most part, bipolar mania constitutes a medical emergency requiring a hospital admission to ensure the immediate safety of the patient or others and rapid symptomatic relief (Keck, British Medical Journal, 327 (7422), 1002-3, 2003).

Bipolar disorders are characterized by cyclical mood changes, ranging from manic to depressed episodes which, early in the illness, can shift suddenly over the course of days. At present, bipolar disorders are treated by maintaining patients on mood-stabilizing therapy (mainly lithium carbonate or anti-epileptics) combined with adjunctive treatment with antidepressants (tricyclic antidepressants or SSRIs) when patients relapse into a depressive episode, or combined with antipsychotics when patients relapse into a manic episode (Liebermann and Goodwin, Curr. Psychiatry Rep. 2004, 6, 459-465). However, the current therapies are associated with several limitations. For example, the use of lithium has a number of disadvantages, including the importance of establishing and maintaining an appropriate concentration of lithium in the blood, as well as being associated with a plethora of physiological conditions including hypothyroidism, tremors, dry mouth, weight gain, increased frequency of urination, nausea, impotence, decreased libido, diarrhea, kidney abnormalities, loss of appetite, visual impairment, seizures and arrhythmias. Additionally, the use of other mainstay drug, valproic acid is associated with hepatic dysfunction. There exists a need to provide an improved therapy in this condition that is devoid of any side-effects.

Psychosis is a generic psychiatric term for mental state in which the components of rational thought and perception are severely impaired. Essentially, a psychotic episode involves loss of contact with reality, often manifest as experiencing suspiciousness, anxiety, fearfulness, paranoia, hallucinations and delusions. It is particularly associated with schizophrenia, bipolar disorder (manic depression), Alzheimer's disease, dementia, Parkinson's disease, severe clinical depression and substance and/or alcohol abuse. The drugs available to treat psychosis (such as atypical antipsychotics) have limited efficacy and produce extrapyramidal symptoms.

This combination of side-effects and limited efficacy of current therapies create a vast unmet need for developing an improved therapeutic treatment for manic episodes, such as for instance manic episodes and psychosis in a subject in need thereof.

### SUMMARY

The inventors of the present disclosure have unexpectedly found that administration of alpha-2 adrenergic agonist, particularly dexmedetomidine or a salt thereof, is a particularly safe and effective intervention for the treatment of conditions such as mania and psychosis that may be associated with various neurological disorders. Further, dexmedetomidine or a salt thereof is unexpectedly safe as there are no associated side effects such as tardive dyskinesia and mania that are associated with conventional antipsychotic therapy. Advantageously, dexmedetomidine acts as a mood stabilizer and thereby producing anti-manic and anti-depressant effects in bipolar patients. The present disclosure discloses that administration of dexmedetomidine via transmucosal route provides a therapeutic utility in the treatment of said disorders.

The present disclosure relates to methods and compositions for treating mania in a subject in need thereof, comprising administering an effective amount of alpha-2 adrenergic agonist or a pharmaceutically acceptable salt thereof to the subject.

In embodiments, there is provided a method of treating mania associated with a diseased condition in a subject in need thereof, comprising administering an effective amount of dexmedetomidine or a pharmaceutically acceptable salt thereof to the subject.

In embodiments, there is provided a method of treating mania associated with neuropsychiatric disorders in a subject in need thereof, comprising administering oromucosally (e.g. sublingually or buccally) an effective amount of dexmedetomidine or a pharmaceutically acceptable salt thereof to the subject.

In embodiments, there is provided a method of treating mania in a subject in need thereof, comprising administering oromucosally (for example, sublingually or buccally) an effective amount of dexmedetomidine or a pharmaceutically acceptable salt thereof to the subject. In embodiments, dexmedetomidine is administered to the subject on a daily basis. In embodiments, dexmedetomidine is administered to the subject at night-time on a daily basis. In embodiments, dexmedetomidine is administered to the subject at night-time as a single dose on a daily basis (e.g. once a day). In embodiments, dexmedetomidine is administered to the subject on a daily basis for one to six times a day. In embodiments, the treatment is effective without causing significant sedation. In embodiments, the treatment is effective without experiencing clinically meaningful cardiovascular effects. In embodiments, the subject is in an agitated state. In embodiments, the subject is in a non-agitated state.

In embodiments, there is provided a method of treating mania in a subject in need thereof, comprising administering oromucosally (for example, sublingually or buccally) an effective amount of dexmedetomidine or a pharmaceutically acceptable salt thereof to the subject wherein said subject is in a non-agitated state. In embodiments, dexmedetomidine is administered to the subject on a daily basis. In embodiments, dexmedetomidine is administered to the subject at night-time on a daily basis. In embodiments, dexmedetomidine is administered to the subject at night-time as a single dose on a daily basis (e.g. once a day). In embodiments, dexmedetomidine is administered to the subject on a daily basis for one to six times a day. In embodiments, the treatment is effective without causing significant sedation. In embodiments, the treatment is effective without experiencing clinically significant cardiovascular effects.

In embodiments, the present disclosure provides an oromucosal composition for treating mania in a subject in need thereof, comprising an effective amount of dexmedetomidine or a pharmaceutically acceptable salt thereof, together with one or more pharmaceutically acceptable excipients and/or carriers. In embodiments, dexmedetomidine is administered to the subject on a daily basis.

In embodiments, the oromucosal composition is selected from the group consisting of a film, patch, lozenge, gel, spray, tablet, liquid drops or the like. In embodiments, the oromucosal composition is film. In embodiments, the composition is a sublingual film. In embodiments, the composition is a buccal film.

In embodiments, there is provided a method of treating mania in a subject in need thereof, comprising administering intramuscularly an effective amount of dexmedetomidine or a pharmaceutically acceptable salt thereof to the subject. In embodiments, dexmedetomidine is administered to the subject on a daily basis. In embodiments, dexmedetomidine is administered to the subject on daily basis one to six times a day. In embodiments, the treatment is effective without causing significant sedation. In embodiments, the treatment is effective without experiencing clinically significant cardiovascular effects. In embodiments, the subject is agitated. In embodiments, the subject is in a non-agitated state. In aspects the subject has dysphoria. In aspects, the subject has mixed mania, where the bipolar patients (1 or 2) experience some symptoms of mania as well as some symptoms of depression

In s embodiments, there is provided a method of treating mania in a subject in need thereof, comprising administering intramuscularly an effective amount of dexmedetomidine or a pharmaceutically acceptable salt thereof to the subject wherein said subject is in a non-agitated state. In embodiments, dexmedetomidine is administered to the subject on a daily basis. In embodiments, dexmedetomidine is administered to the subject on daily basis for one to six times a day. In embodiments, the treatment is effective without causing significant sedation. In embodiments, the treatment is effective without experiencing clinically significant cardiovascular effects. In embodiments, the present disclosure provides an intramuscular composition for treating mania in a subject in need thereof, comprising an effective amount of dexmedetomidine or a pharmaceutically acceptable salt thereof, together with one or more pharmaceutically acceptable excipients and/or carriers. In embodiments, dexmedetomidine is administered to the subject on a daily basis.

Bipolar 1 patients with mania have manic episodes that cycle with depression. In contrast, bipolar 2 patients suffer hypomanic episodes, but both Bipolar 1 and Bipolar 2 tend to cycle into depressive episodes. (Goodwin & Jameson, 2d Ed. Bipolar Disorders) A diagnostic characteristic of manic and hypomanic mood episodes is the associated sleep disruption. Patients report and experience a decreased need for sleep; often sleeping only a few hours, if at all, at night. Previous approaches have used dexmedetomidine to treat agitation associated with these disorders, and the present disclosure is believed to be the first showing that the underlying disorders can be treated in a non-agitated subject. Advantageously, the methods and compositions disclosed herein can be used as mood stabilizers. Mood stabilizers are medicines that reduce or prevent the highs (mania) and lows (depression) of bipolar disorders. Other mood stabilizers, such as lithium and valproic acid are known, and typically are co-administered with anti-psychotics, such as lurasidone. But these approaches often induce or promote mania or depression, the opposite outcome of that sought here, or they do not work at all. In other approaches, anti-depressants may be administered to bipolar patients. Again, this can enhance or produce, rather than treat mania. In addition, dexmedetomidine normalizes depression in the patient. Finally, after treatment of subjects with dexmedetomidine over extended periods such as a week, month or longer, the subjects may also exhibit improved sleep architecture, providing an additional advantageous therapeutic benefit not observed with traditional approaches to treating mania or bipolar disorders. Dexmedetomidine is thus superior to other mood stabilizers because the mania is treated rather than enhanced, depression is alleviated, and the sleep architecture is improved (including increased time spend in restorative deep sleep), which may help prevent episodes of mood alteration (mood stabilization); either elevation (mania, hypomania or mixed mania) or depression (depressive episodes).

In embodiments, there is provided a method of treating hypomania associated with bipolar disorder II, (also referred to as bipolar disorder 2) in a subject in need thereof, comprising administering oromucosally an effective amount of dexmedetomidine or a pharmaceutically acceptable salt thereof to the subject. In embodiments, dexmedetomidine is administered to the subject on a daily basis. In embodiments, dexmedetomidine is administered to the subject at night-time on a daily basis (e.g. once a day). In embodiments, dexmedetomidine is administered to the subject at night-time as a single dose on a daily basis. In embodiments, dexmedetomidine is administered to the subject on a daily basis for one to six times a day. In embodiments, the subject is in a non-agitated state.

In embodiments, there is provided a method of treating bipolar mania in a subject in need thereof, comprising administering oromucosally (for example, sublingually or buccally) an effective amount of dexmedetomidine or a pharmaceutically acceptable salt thereof to the subject. In embodiments, dexmedetomidine is administered to the subject on a daily basis. In embodiments, the treatment is effective without causing significant sedation. In embodiments, the treatment is effective without experiencing clinically significant cardiovascular effects. In embodiments, the subject is in a non-agitated state. In embodiments, the subject is in an agitated state.

The disclosure provides methods and compositions for treating psychosis in a subject in need thereof, comprising administering an effective amount of alpha-2 adrenergic agonist or a pharmaceutically acceptable salt thereof to the subject.

In embodiments, there is provided a method of treating psychosis in a subject in need thereof, comprising administering an effective amount of dexmedetomidine or a pharmaceutically acceptable salt thereof to the subject.

In embodiments, there is provided a method of treating psychosis in a subject in need thereof, comprising administering oromucosally (sublingually or buccally) an effective amount of dexmedetomidine or a pharmaceutically acceptable salt thereof to the subject. In embodiments, dexmedetomidine is administered to the subject on a daily basis. In embodiments, dexmedetomidine is administered to the subject at night-time on a daily basis. In embodiments, dexmedetomidine is administered to the subject at night-time as a single dose on a daily basis. In embodiments, dexmedetomidine is administered to the subject on a daily basis for one to six times a day. In embodiments, the treatment is effective without causing significant sedation. In embodiments, the treatment is effective without experiencing clinically significant cardiovascular effects. In embodiments, the subject is in a non-agitated state

In embodiments, the present disclosure provides an oromucosal composition for treating psychosis in a subject in need thereof, comprising an effective amount of dexmedetomidine or a pharmaceutically acceptable salt thereof, together with one or more pharmaceutically acceptable excipients and/or carriers.

In embodiments, the oromucosal composition is selected from the group consisting of a film, patch, lozenge, gel, spray, tablet, liquid drops or the like. In embodiments, the oromucosal composition is film. In embodiments, the composition is a sublingual film. In embodiments, the composition is a buccal film.

In embodiments, there is provided a method of treating psychosis in a subject in need thereof, comprising administering intramuscularly an effective amount of dexmedetomidine or a pharmaceutically acceptable salt thereof to the subject. In embodiments, dexmedetomidine is administered to the subject on a daily basis wherein the subject is in a non-agitated state.

In embodiments, dexmedetomidine is administered to the subject on a daily basis for one to six times a day. In embodiments, the treatment is effective without causing significant sedation. In embodiments, the treatment is effective without experiencing clinically significant cardiovascular effects.

In embodiments, the present disclosure provides an intramuscular composition for treating psychosis in a subject in need thereof, comprising an effective amount of dexmedetomidine or a pharmaceutically acceptable salt thereof, together with one or more pharmaceutically acceptable excipients and/or carriers.

In embodiments, the psychosis is acute. In embodiments, the psychosis is chronic. In embodiments, the psychosis is associated with a disease condition such as neuropsychiatric disease or disorder; for example, schizophrenia, schizoaffective disorder, depression, dementia and bipolar disorder (e.g. bipolar I disorder and bipolar II disorder), optionally the dementia or mood disorder in a subject with major depressive episode or another related neuropsychiatric disorder.

In embodiments, the psychosis is associated with diseased condition such as substance abuse disorders (e.g, alcohol, opioid and other substance withdrawal). In embodiments, psychosis is associated with depression. In embodiments, psychosis is associated with schizophrenia. In embodiments, psychosis is associated with bipolar disorder. In embodiments, psychosis is associated with dementia. In embodiments, psychosis is associated with Parkinson's disease. In embodiments, the subject is in an agitated state. In embodiments, the subject is in a non-agitated state.

In embodiments, there is provided a method of treating psychosis associated with neuropsychiatric disorders in a subject in need thereof, comprising administering oromucosally (sublingually or buccally) an effective amount of dexmedetomidine or a pharmaceutically acceptable salt thereof to the subject wherein said subject is in a non-agitated state.

In embodiments, there is provided a method of treating psychosis associated with neurodegenerative disorders in a subject in need thereof, comprising administering oromucosally (sublingually or buccally) an effective amount of dexmedetomidine or a pharmaceutically acceptable salt thereof to the subject wherein said subject is in a non-agitated state.

In embodiments of the disclosure, the dosage of dexmedetomidine administered oromucosally may conveniently be in the range of about 2 μg to about 300 μg. Examples of suitable dosages include: about 2 μg to about 250 μg, about 2 μg to about 200 μg, about 2 μg to about 190 μg, about 2 μg to about 180 μg, about 3 μg to about 170 μg, about 3 μg to about 160 μg, about 3 μg to about 150 μg, about 4 μg to about 140 μg, about 4 μg to about 120 μg, about 5 μg to about 100 μg, about 5 μg to about 90 μg, about 5 μg to about 85 μg, about 5 μg to about 80 μg, about 5 μg to about 75 μg, about 5 μg to about 70 μg, about 5 μg to about 65 μg, about 5 μg to about 60 μg, about 5 μg to about 55 μg, about 5 μg to about 50 μg, about 5 μg to about 45 μg, about 5 μg to about 40 μg, about 5 μg to about 35 μg, about 5 μg to about 30 μg, about 5 μg to about 25 μg, about 5 μg to about 20 μg, about 5 μg to about 15 μg, about 5 μg to about 10 μg, less than 10 micrograms (e.g. about 5, 6, 7, 8, or 9 micrograms), about 10 μg, about 12 μg, about 14 μg, about 15 μg, about 16 μg, about 18 μg, about 20 μg, about 30 μg, about 50 μg. The dose may be administered one or more times a day. The doses can be administered daily for longer period of time for at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 2 months, at least about 3 months, at least about 4 months, at least about 5 months, at least about 6 months or at least about one year. In embodiments, the dose is administered for about 2 weeks to about 4 weeks followed by conventional antipsychotic or standard-of-care (SOC).

### BRIEF DESCRIPTION OF THE DRAWINGS

FIG. 1: depicts the improvement in YMRS scale in bipolar disorder patients at 24 hours following administration of 180 μg dexmedetomidine hydrochloride sublingual film. Asterisks showed significance versus placebo at 24 hours. *p<0.05; ** p<0.01 and *** p<0.005 FIG. 2A: depicts the Smart-cube signatures of dexmedetomidine hydrochloride (at different doses) similar to antipsychotic drugs.

FIG. 2B: depicts the Smart-cube signatures of two alpha 2-adrenergic receptors agonists (guanfacine and clonidine).

### DETAILED DESCRIPTION

### Abbreviations

mcg or μg—Microgram
μg—Microgram
FTD: fronto-temporal dementia
Dex or DEX: Dexmedetomidine
DLB: dementia with Lewy bodies
DT: disintegration time
FDA: Food and Drug Administration
HPC: Hydroxypropyl cellulose
HPMC: hydroxypropyl methylcellulose
IM: intramuscular
MW: Molecular weight
mm: Millimeter
MACS: Mania Acute Rating Scale
PEO: Polyethylene oxide
PANSS: Positive and Negative Syndrome Scale
RASS: Richmond Agitation Sedation Scale
SC: Smartcube
YMRS: Young Mania Rating Scale

### Definitions

As used herein, “about” means plus or minus 10% of the indicated numerical value. Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The terms “dosage form” or “pharmaceutical composition” or “formulation” or “composition of the disclosure,” and “composition” are used interchangeably, except where otherwise clearly intended to have different meanings.

The term “a” or “an” refers to one or more of that entity. As such, the terms “a” (or “an”), “one or more” and “at least one” are used interchangeably herein. In addition, reference to “an agent” by the indefinite article “a” or “an” does not exclude the possibility that more than one of the agents are present, unless the context clearly requires that there is one and only one of the agents.

The terms “comprises”, “comprising”, “includes”, “including”, “having” means “including but not limited to”. The present invention may suitably “comprise”, “consist of”, or “consist essentially of”, the steps, elements, and/or reagents described in the claims.

As used herein, “pharmaceutically acceptable salt” refers to a salt known to be non-toxic and commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenyl substituted alkanoic acids, hydroxyalkanoic and hydroxyl alkandioic acids, aromatic acids, aliphatic and aromatic sulfonic acids may also be used. A preferred salt is hydrochloride (or dihydrochloride) salt.

The term “pharmaceutically acceptable carrier” refers to a pharmacologically inert substance to be used as a carrier. As used herein, the phrase “carrier” and “excipients” are used interchangeably, except where otherwise clearly intended to have different meanings.

The term “without significant sedation” and the like means that the patient experiences a level of sedation not greater than Level 3 on the Ramsay Sedation Scale. Level 3 means sedated but responds to commands. In some embodiments, the dexmedetomidine may be dosed to achieve a Richmond Agitation Sedation Scale (RASS) of −1 (“light sedation”).

The term “an effective amount” is interchangeable with “therapeutically effective dose,” or “therapeutically effective amount,” and refers to an amount sufficient to produce the desired effect. An effective amount is sufficient to cause an improvement in a condition of the subject.

The terms “treat”, “treating” or “treatment” in reference to a particular disease or disorder includes lessening, improving, ameliorating or abrogating the symptoms and/or pathology of the disease or disorder. The term “prevention” means preventing the occurrence of a disease, condition, or associated symptoms or preventing the recurrence of the same, for example after a period of improvement.

The term “mania” refers to a psychological condition that causes a person to experience unreasonable euphoria, very intense moods, hyperactivity, and delusions. Mania (or manic episodes) is a common symptom of bipolar disorder. A different form of mania is called “hypomania” that lasts for a short period (usually a few days). According to DSM-5 criteria, hypomania is distinct from mania in that there is no significant functional impairment; mania, by DSM-5 definition, does include significant functional impairment and may have psychotic features. A patient with mania may also be said to be having a manic episode, which reflect the cyclical nature of the disorder.

The term “psychosis” refers to a range of conditions that affect the mind, in which there has been some loss of contact with reality. Psychosis is characterized by significant changes in a person's perceptions, thoughts, beliefs, and behaviors. Symptoms may include delusions (false beliefs) and hallucinations (seeing or hearing things that others do not see or hear). Psychosis is a symptom associated with a number of health conditions including the manic phase of bipolar I disorder, as well as schizophrenia, post-traumatic stress disorder (PTSD), and schizoaffective disorder.

As used herein, the term “subject” preferably refers to a human patient. In some embodiments, the subject can be any animal, including non-human mammals, such as mice, rats, other rodents, rabbits, dogs, cats, swine, cattle, sheep, horses, or primates.

The term “oromucosal” means administration to the oral mucosa, specifically the oral cavity and/or the pharynx. Oromucosal administration includes sublingual and buccal routes.

The term “sublingual” means “under the tongue” and refers to a method of administering substances via the blood vessels under the tongue. Sublingual absorption occurs through the highly vascularized sublingual mucosa, which allows a substance direct access to the blood circulation, thereby providing for direct systemic administration independent of gastrointestinal influences and avoiding undesirable first-pass hepatic metabolism.

The term “buccal” means administration of the dosage form against the gum and the inner lip or cheek.

The term “film” herein includes thin films, in any shape, including rectangular, square, or other desired shape. The film may be of any desired thickness and size, such that it can be conveniently placed sub-lingually in the patient. For example, the film may be a thin film having a thickness of from about 20 micrometers to about 200 micrometers or may be a thick film having a thickness of from about 20 micrometers to about 1000 micrometers. In embodiments, the film may have a thickness greater than about 1000 micrometers.

The term “dissolvable” means the films herein are readily disintegrated, e.g. at least within about 20 minutes, following administration to the oral mucosa. Disintegration is achieved by saliva and/or other aqueous materials on the mucosal surface.

The term “mucoadhesion” is used herein to refer to adhesion to mucosal membranes, such as those in the oral cavity.

The term “mucoadhesive” refers to the property of adhering to a mucosal tissue surface in vivo. Such adhesion adherently localizes the dosage form onto the mucus membrane and requires the application of a force to separate the mucoadhesive material from the mucus membrane.

“Therapeutic” as used herein, refer to treatment and/or prophylaxis, depending on context.

The term “neurodegenerative disorders” means diseases that feature neurodegeneration and include, but is not limited to, Alzheimer's disease, frontotemporal dementia (or Pick's disease), Dementia (e.g., Dementia with Lewy bodies, vascular dementia), posttraumatic stress disorder (PTSD), Parkinson's disease, vascular cognitive impairment, Huntington's disease, multiple sclerosis, Creutzfeldt-Jakob disease, multiple system atrophy, progressive supranuclear palsy or Amyotrpohic Lateral Sclerosis (ALS, or Lou Gehrig's Disease).

The term “neuropsychiatric disorders” includes, but is not limited to, schizophrenia, bipolar illness (e.g., bipolar disorder 1 or 2), cyclothymia, depression (major depressive episodes in bipolar disorder and in major depressive disorder), and delirium.

“Opioid or alcohol or substance withdrawal” refers to a variety of signs and complaints appearing with the abrupt removal of, or a rapid decrease in the regular dosage of, opioids or alcohol or other drug substances. Physical manifestations may include sweating, nausea, yawning, chills, diarrhea, papillary dilation, piloerection, tachycardia, increased blood pressure, hypersensitivity to pain, stomach cramps, and muscle cramps.

As used herein, the phrase “water-soluble polymer” refers to (i) a polymer that is at least partially soluble in water, and desirably fully or predominantly soluble in water, and/or (ii) a polymer that absorbs water. Polymers that absorb water are referred to herein as water-swellable polymers.

As used herein, the phrase “disposed within a polymer matrix” means that dexmedetomidine or a pharmaceutically acceptable salt thereof is incorporated directly into the polymer solution prior to the formation of the solid polymer matrix film composition.

As used herein, the phrase “deposited on the surface of a polymer matrix” means that dexmedetomidine or a pharmaceutically acceptable salt thereof is formulated as liquid composition separate from the preparation of the solid polymer matrix, and deposited onto the solid polymer, e.g. as one or more micro-deposits, where it dries or is dried. The dried product is sometimes referred to herein as the “micro-deposited matrix film”. The drug liquid formulation may be in any form, including as a solution, emulsion, suspension, or dispersion.

The term “unit dose,” “unit dosage,” or “unit dosage form” means a physically discrete unit that contains a predetermined quantity of dexmedetomidine or a pharmaceutically acceptable salt thereof.

The term “parenteral” refers to administration of a drug by injection under one or more layer of skin or mucous membrane, and can include, for example, subcutaneous, intravenous, intraperitoneal or intramuscular injection.

The term“clinically significant cardiovascular effects” means herein a lowering in blood pressure (hypotension) and/or heart rate (bradycardia) to the extent that medical intervention is required to address the cardiovascular side effects, where the term “medical intervention” means an intervention that more serious than administering fluids, such as an energy drink.

### Active Agent

Dexmedetomidine has the IUPAC name (+) 4-(S)-[1-(2,3-dimethylphenyl)ethyl]-1H-imidazole. As the monohydrochloride salt, it is predominantly used as a medication for the sedation of patients during treatment in an intensive care setting or to sedate patients prior to and/or during surgical and other procedures. Such medication is currently sold under the registered trade name “PRECEDEX”.

Pharmaceutically acceptable salts of dexmedetomidine that may be used herein include generally any suitable salt that has been or may be approved by the US FDA or other appropriate foreign or domestic agency for administration to a human. Non-limiting examples of suitable pharmaceutically acceptable salts include salts of inorganic acids such as hydrochloric, hydrobromic, nitric, carbonic, monohydrocarbonic, phosphoric, monohydrogen phosphoric, dihydrogen phosphoric, sulfuric, hydrogen sulfuric, and hydroiodic acid. Other examples include salts derived from non-toxic organic acids, including acetic, propionic, isobutyric, maleic, malonic, benzoic, succinic, suberic, fumaric, lactic, mandelic, phthalic, benzenesulfonic, p-toluenesulfonic, citric, tartaric, and methanesulfonic acids, or combinations of these acid salts. Exemplary salts include dexmedetomidine hydrochloride, dexmedetomidine hydrobromide, dexmedetomidine sulfate, dexmedetomidine sulfonate, dexmedetomidine phosphate, dexmedetomidine nitrate, dexmedetomidine formate, dexmedetomidine citrate, dexmedetomidine tartrate, dexmedetomidine malate, dexmedetomidine benzoate, dexmedetomidine salicylate, dexmedetomidine ascorbate or the like. In other embodiments, deuterated forms of dexmedetomidine or a pharmaceutically acceptable salt thereof may be included.

### II. Dosage

In embodiments, the dosage of dexmedetomidine or a pharmaceutically acceptable salt thereof is in the range of between about 10 μg to about 405 μg, about 0.5 μg to about 300 μg. Examples of suitable dosages include: about 0.5 μg to about 280 μg, about 1 μg to about 270 μg, about 1 μg to about 260 μg, about 1 μg to about 250 μg, about 1 μg to about 240 μg, about 1 μg to about 230 μg, about 1 μg to about 220 μg, about 1 μg to about 210 μg, about 1 μg to about 200 μg, about 1 μg to about 190 μg, about 1 μg to about 180 μg, about 1 μg to about 170 μg, about 1 μg to about 160 μg, about 1 μg to about 150 μg, about 1 μg to about 140 μg, about 1 μg to about 130 μg, about 1 μg to about 120 μg, about 1 μg to about 110 μg, about 1 μg to about 100 μg, about 3 μg to about 90 μg, about 3 μg to about 80 μg, about 3 μg to 70 μg, from about 3 μg to about 60 μg, from about 3 μg to 50 μg, from about 3 μg to about 40 μg, from about 3 μg to about 35 μg, from about 5 μg to about 35 μg, about 10 μg to about 50 μg, about 10 μg to about 40 μg, about 10 μg to about 35 μg or about 15 μg to 35 μg. The dose may be administered one or more times a day including twice, three times, four times, five times or six times per day.

In embodiments, dexmedetomidine or a pharmaceutically acceptable salt thereof may be administered oromucosally (e.g. sublingually or buccally at a dose of about 10 μg to about 405 μg, about 10 μg to about 400 μg, about 10 μg to about 350 μg, about 10 μg to about 300 μg, about 10 μg to about 270 μg, about 20 μg to about 240 μg, about 30 μg to about 180 μg, about 40 μg to about 140 μg, about 50 μg to about 120 μg, about 60 μg to about 120 μg, about 70 μg to about 100 μg, about 80 μg to about 100 μg, about 100 μg to about 200 μg, about 120 μg to about 200 μg, or about 120 μg to about 180 μg. In embodiments, dexmedetomidine or a pharmaceutically acceptable salt thereof may be administered intramuscularly at a dose of about 10 μg to about 405 μg, about 10 μg to about 400 μg, about 10 μg to about 350 μg, about 10 μg to about 300 μg, about 10 μg to about 270 μg, about 20 μg to about 240 μg, about 30 μg to about 180 μg, about 40 μg to about 140 μg, about 50 μg to about 120 μg, about 60 μg to about 120 μg, about 70 μg to about 100 μg, about 80 μg to about 100 μg, about 100 μg to about 200 μg, about 120 μg to about 200 μg, or about 120 μg to about 180 μg.

In embodiments, dexmedetomidine or a pharmaceutically acceptable salt thereof may be administered oromucosally (e.g. sublingually or buccally) or intramuscularly at a dose of about (in μg): about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245 or about 250. In embodiments, the dexmedetomidine or a pharmaceutically acceptable salt thereof may be administered oromucosally (e.g. sublingually or buccally) as a film. The film may be a film described in U.S. Pat. No. 10,792,246, which is hereby incorporated by reference in its entirety for all purposes. In embodiments, the film is administered as a single unit dose comprising about 10 μg to about 405 μg or about 100 μg to about 200 μg. In embodiments, each unit contains at least one spot of micro-deposited dexmedetomidine or a pharmaceutically acceptable salt thereof. In embodiments, each unit contains two or more spots of micro-deposited dexmedetomidine or a pharmaceutically acceptable salt thereof. For example, for a film having a unit dose of 120 μg of dexmedetomidine hydrochloride, the film may have one micro-deposited spot comprising 120 μg of dexmedetomidine hydrochloride or it may have two micro-deposited spots comprising 60 μg of dexmedetomidine hydrochloride. Similarly, for a film having a unit dose of 180 μg of dexmedetomidine hydrochloride, the film may have one micro-deposited spot comprising 180 μg of dexmedetomidine hydrochloride or it may have two micro-deposited spots comprising 90 μg of dexmedetomidine hydrochloride. In embodiments, one or more unit doses are administered to deliver the total dose.

In embodiments, dexmedetomidine or a pharmaceutically acceptable salt thereof may be administered oromucosally (e.g. sublingually or buccally) at a dose of about 120 μg to about 405 μg, e.g. about 120 μg to about 270 μg, including about 120 μg and about 180 μg. In embodiments, these doses can be provided via one or more unit dosage forms to deliver the total dose. Examples of suitable doses include (in μg): about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400 and about 405.

In embodiments, dexmedetomidine or a pharmaceutically acceptable salt thereof may be administered oromucosally (e.g. sublingually or buccally) at a dose of about 10 μg to about 200 μg, e.g. about 120 μg to about 190 μg. Examples of suitable doses include (in μg): about 10, about 20, about 30, about 40, about 50, about 60, about 70, about 80, about 90, about 100, about 105, about 110, about 115, about 120, about 125, about 130, about 135, about 140, about 145, about 150, about 155, about 160, about 165, about 170, about 175, about 180, about 185, about 190, about 195 and about 200.

In embodiments, dexmedetomidine or a pharmaceutically acceptable salt thereof may be administered sublingually at a dose of about 10 μg to about 200 μg, e.g. about 120 μg to about 190 μg. Examples of suitable doses include (in μg): about 10, about 20, about 30, about 40, about 50, about 60, about 70, about 80, about 90, about 100, about 105, about 110, about 115, about 120, about 125, about 130, about 135, about 140, about 145, about 150, about 155, about 160, about 165, about 170, about 175, about 180, about 185, about 190, about 195 and about 200.

In embodiments, dexmedetomidine or a pharmaceutically acceptable salt thereof may be administered buccally at a dose of about 10 μg to about 200 μg, e.g. about 120 μg to about 190 μg. Examples of suitable doses include (in μg): about 10, about 20, about 30, about 40, about 50, about 60, about 70, about 80, about 90, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295 and about 300.

In embodiments, dexmedetomidine or a pharmaceutically acceptable salt thereof may be administered intramuscularly at a dose of about 10 μg to about 200 μg, e.g. about 120 μg to about 190 μg. Examples of suitable doses include (in μg): about 10, about 20, about 30, about 40, about 50, about 60, about 70, about 80, about 90, about 100, about 105, about 110, about 115, about 120, about 125, about 130, about 135, about 140, about 145, about 150, about 155, about 160, about 165, about 170, about 175, about 180, about 185, about 190, about 195, about 200, about 200, about 205, about 210, about 215, about 220, about 225, about 230, about 235, about 240, about 245, about 250, about 255, about 260, about 265, about 270, about 275, about 280, about 285, about 290, about 295 and about 300.

In embodiments, dexmedetomidine or a pharmaceutically acceptable salt thereof (e.g. dexmedetomidine hydrochloride) is administered in an amount of about 180 μg.

In embodiments, dexmedetomidine or a pharmaceutically acceptable salt thereof (e.g. dexmedetomidine hydrochloride) is administered in an amount of about 120 μg.

In embodiments, dexmedetomidine or a pharmaceutically acceptable salt thereof (e.g. dexmedetomidine hydrochloride) is administered in an amount of about 90 μg.

In embodiments, dexmedetomidine or a pharmaceutically acceptable salt thereof (e.g. dexmedetomidine hydrochloride) is administered in an amount of about 60 μg.

In embodiments, dexmedetomidine or a pharmaceutically acceptable salt thereof (e.g. dexmedetomidine hydrochloride) is administered in an amount of about 40 μg.

In embodiments, dexmedetomidine or a pharmaceutically acceptable salt thereof (e.g. dexmedetomidine hydrochloride) is administered in an amount of about 30 μg.

In embodiments, the dosage of dexmedetomidine or a pharmaceutically acceptable salt thereof may be administered twice a day. In embodiments, the dosages of dexmedetomidine or a pharmaceutically acceptable salt thereof are administered at a dose of about 30 μg to about 90 μg during daytime (e.g., 30 μg, 45 μg, 60 μg, or 90 μg) and about 120 μg to about 180 μg during night-time (e.g., 120 μg or 180 μg). In embodiments, the dosages of dexmedetomidine or a pharmaceutically acceptable salt thereof are administered at a dose of about 30 μg to about 90 μg during daytime and 30 μg to about 90 μg during night-time. In embodiments, the dosages of dexmedetomidine or a pharmaceutically acceptable salt thereof are administered twice a day at a dose of about 120 μg to about 180 μg during daytime and about 30 μg to about 90 μg during night-time.—In embodiments, dexmedetomidine or a pharmaceutically acceptable salt thereof is administered as 60 μg per unit dose twice a day to a total dose of 120 μg. For example, a 60 μg unit dose is taken in the morning and another 60 μg unit dose is taken in the evening or night-time.

The exemplary dosage of dexmedetomidine or a pharmaceutically acceptable salt thereof to be administered to a particular patient, will depend on the type and extent of the condition, the overall health status of the particular patient, the particular form of dexmedetomidine or a pharmaceutically acceptable salt thereof being administered, and the particular formulation used to treat the patient.

### III. Pharmaceutical Compositions

According to the present disclosure, dexmedetomidine or a pharmaceutically acceptable salt thereof can be administered to the human subject through various routes, including oromucosal (e.g. sublingual or buccal), oral, parenteral and the like. Formulations suitable for use according to the present disclosure are outlined below. Additional formulations suitable for use according to the present disclosure are described in U.S. Pat. No. 10,792,246, which is hereby incorporated by reference in its entirety for all purposes.

Oromucosal Formulations (Sublingual and/or Buccal Formulations)

Dexmedetomidine or a pharmaceutically acceptable salt thereof can be formulated, according to the present disclosure, into dosage forms suitable for oromucosal administration. Such dosage forms include tablets, powders, pills, films, capsules, liquids, gels, syrups, slurries, suspensions, and the like. In embodiments, dexmedetomidine or a pharmaceutically acceptable salt thereof is formulated as a film product.

Carriers suitable for inclusion in oromucosal (e.g. sublingual or buccal) formulations include, but are not limited to, sugars, starches, cellulose and its derivatives, malt, gelatin, talc, calcium sulphate, vegetable oils, synthetic oils, polyols, alginic acid, phosphate buffered solutions, emulsifiers, isotonic saline, pyrogen—free water and combinations thereof. Carriers which readily dissolve in saliva may be preferred.

Oromucosal (e.g. sublingual or buccal) formulations may also include other pharmaceutically acceptable carriers and/or excipients such as binders, lubricants, diluents, coatings, disintegrants, barrier layer components, glidants, colouring agents, solubility enhancers, gelling agents, fillers, proteins, co-factors, emulsifiers, solubilising agents, suspending agents and mixtures thereof. Particular excipients, which may be used according to this disclosure, are known in the art, for example as described in Handbook of Pharmaceutical Excipients, fifth edition, 2005 edited by Rowe et al., Mcgraw Hill.

Films

Suitable films for oromucosal (e.g. sublingual or buccal) administration according to the present disclosure comprise dexmedetomidine or a pharmaceutically acceptable salt thereof either (i) disposed within a polymer matrix or (ii) deposited on the surface of a polymer matrix, e.g., on the surface of a “placebo” film.

### Polymer Component of Film

The polymer component may be one or more water-soluble polymers within the film matrix and/or as part of the drug-containing deposit (e.g. one or more droplets) on the surface of the polymer. In embodiments of the disclosure, the polymer component consists of a single water-soluble polymer. In embodiments, the polymer component consists of two or more water-soluble polymers, including two or more of the same water-soluble polymers having different molecular weights.

The polymer component in the film matrix is of a suitable composition and present in a sufficient amount to ensure rapid disintegration of the film matrix in the oral mucosa. For example, the presence of the polymer component may allow the film matrix to disintegrate completely oromucosally in about 15 seconds to about 180 seconds, for example, about 30 seconds to about 180 seconds, including about 120 seconds. The polymer component in the film matrix also provides the film with sufficient strength (i.e. the film is self-supporting).

When present in one or more droplets of the dexmedetomidine composition deposited onto the surface of the polymer matrix/substrate, the polymer component may, for example, consist of the water-soluble polymer hydroxypropyl cellulose, although different water-soluble polymers are also contemplated as described hereinafter under the definition “first water-soluble polymer” and “second water soluble polymer”. For example, the polymer component may consist of one, two or three hydroxypropyl celluloses having different molecular weights. The molecular weights of the different hydroxypropyl celluloses may conveniently range from (i) less than about 60,000 daltons (e.g. about 5,000 daltons to about 49,000 daltons) (ii) about 90,000 daltons to about 200,000 daltons and (iii) about 200,000 daltons to about 500,000 daltons. The two or more hydroxypropyl celluloses may be mixed in any suitable ratio to achieve the desired droplet viscosity. The viscosity of the dexmedetomidine composition solution or suspension can be measured using a Brookfield viscometer with a small sample adapter at a temperature of 25° C. and may range from about 5 cps to about 3700 cps. For example, it may range from about 5 cps to about 500 cps, about 6 cps to about 200 cps, about 6 cps to about 100 cps or about 6 cps to about 50 cps. In embodiments of the present disclosure, the viscosity of the dexmedetomidine composition solution or suspension is from about 6 cps to about 20 cps at 25° C. and a shear rate of about 7 (1/s).

When present in a monolithic (i.e. placebo or drug-containing) film, the polymer component may, for example, consist of one water soluble polymer or two different water-soluble polymers. When two different water-soluble polymers are present, one of the water-soluble polymers may include the same polymer but present in the polymer component as a combination of different molecular weights. For example, the polymer component may consist of one, two or three hydroxypropyl celluloses having different molecular weights, although different water-soluble polymers are also contemplated as described hereinafter under the definition “first water-soluble polymer” and “second water soluble polymer” such as polyethylene oxide. The molecular weights of the different hydroxypropyl celluloses may conveniently range from (i) less than about 60,000 daltons (e.g. about 5000 daltons to about 49000 daltons) (ii) about 90000 daltons to about 200,000 daltons and (iii) about 200,000 daltons to about 500,000 daltons (e.g. about 300000 daltons to about 450000 daltons). The two or more hydroxypropyl celluloses (e.g. low and high molecular weight hydroxypropyl celluloses) may be mixed in any suitable ratio to achieve the desired film properties. When present in a monolithic (i.e. placebo or drug-containing) film or micro-deposited film matrix composition, the polymer component may conveniently consist of one or more water-soluble polymers having a molecular weight less than about 60,000 daltons (e.g. about 5,000 daltons to about 49,000 daltons), and/or from about 90000 daltons to about 200,000 daltons and/or about 200,000 daltons to about 500,000 daltons (e.g. about 300000 daltons to about 450000 daltons). When a structurally different water-soluble polymer is also present, it may conveniently have a higher molecular weight, for example a molecular weight greater than about 500,000 daltons.

In embodiments, the disclosure provides pharmaceutical film compositions, comprising: (i) dexmedetomidine or a pharmaceutically acceptable salt thereof; (ii) a polymer component consisting of a first water-soluble polymer having a molecular weight less than about 60,000 daltons (e.g. about 5,000 daltons to about 49,000 daltons), and one or more second-water soluble polymers having a molecular weight greater than about 60,000 daltons; and, optionally, (iii) one or more pharmaceutically acceptable carriers.

In embodiments, the disclosure provides pharmaceutical film compositions consisting essentially of: (i) dexmedetomidine or a pharmaceutically acceptable salt thereof, (ii) a polymer component consisting of a first water-soluble polymer having a molecular weight less than about 60,000 daltons (e.g. about 5,000 daltons to about 49,000 daltons), and one or more second-water soluble polymers having a molecular weight greater than about 60,000 daltons; and, optionally, (iii) one or more pharmaceutically acceptable carriers.

In embodiments, the disclosure provides pharmaceutical film compositions consisting of: (i) dexmedetomidine or a pharmaceutically acceptable salt thereof; (ii) a polymer component consisting of a first water-soluble polymer having a molecular weight less than about 60,000 daltons (e.g. about 5,000 daltons to about 49,000 daltons), and one or more second water-soluble polymers having a molecular weight greater than about 60,000 daltons; and, optionally, (iii) one or more pharmaceutically acceptable carriers.

Examples of one or more first water-soluble polymers are selected from the group consisting of hydroxypropyl cellulose (HPC), hydroxyethyl cellulose, hydroxypropyl methylcellulose (HPMC), carboxymethyl cellulose, methyl cellulose and mixtures thereof, including mixtures of the same polymer having different molecular weights.

Examples of one or more second water-soluble polymers are selected from the group consisting of hydroxypropyl cellulose, hydroxypropyl methylcellulose, hydroxyethyl cellulose, carboxy methylcellulose, methylcellulose and mixtures thereof, including mixtures of the same polymer having different molecular weights. Polyethylene oxide (PEO) may also be present herein as a second water-soluble polymer or may be described separately hereinafter in the pharmaceutical film compositions as an example of a pharmaceutically acceptable carrier, or more particularly, as a mucoadhesive agent.

In embodiments, the weight ratio of said first water-soluble polymer to said second water-soluble polymer(s) (including PEO when present in the film) in the entire film composition is from about 2:1 to about 1:50, for example about 1:1 to about 1:40, including about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40.

In embodiments, the weight ratio of said first water-soluble polymer to said second water-soluble polymer(s) (including PEO when present in the film) in the entire film composition is from about 1:10 to about 1:30, about 1:15 to about 1:25 or about 1:15 to about 1:20. In embodiments, a ratio of about 1:15 to about 1:20 provides beneficial functional effects.

Examples of other water-soluble polymers which may be included in the film with the first water-soluble polymer/second water-soluble polymer or replace such polymer(s) include povidone (polyvinylpyrrolidone), copovidone (copolymers of N-vinyl-2-pyrrolidone and vinyl acetate), polyvinyl alcohol, polyethylene glycol, polyacrylic acid, methylmethacrylate copolymer, carboxyvinyl copolymers, polydextrose, pullulan, carboxymethyl cellulose, sodium alginate, chitosan, xanthan gum, tragacanth gum, guar gum, acacia gum, arabic gum, starch, carrageenan, gelatin and mixtures thereof. The water-soluble polymer component, including water-soluble polymer carriers when present, may conveniently comprise about 40% to about 99.8%, about 50% to about 99.7%, about 60% to about 99.6% of the film composition, based on the weight of the film on a dry weight basis.

In embodiments, the polymer component for the film composition comprises a first water-soluble polymer present in an amount of from about 2% to about 15% on a dry weight basis of the polymer component (e.g. at about 3% to about 8% w/w of the total film weight). This water-soluble polymer may conveniently have a molecular weight from about 5,000 daltons to about 49,000 daltons. Examples of suitable such water-soluble polymers include those selected from the group consisting of hydroxypropyl cellulose, hydroxyethyl cellulose, hydroxypropyl methylcellulose, carboxymethyl cellulose, methyl cellulose, and mixtures thereof.

In embodiments, low molecular weight hydroxypropyl cellulose may be present in the film at about 3% to about 8% w/w of the total film weight.

In embodiments, the one or more second water-soluble polymers (including water-soluble polymer carriers such as polyethylene oxide) may, for example, be present in an amount of from about 50 to about 98 weight percent on dry weight basis of the polymer component. The one or more second water-soluble polymers each has a molecular weight greater than 60,000 daltons; for example, from about 90,000 daltons to about 1,500,000 daltons, especially when the polymer is selected from the group consisting of polyethylene oxide, hydroxypropyl cellulose, hydroxypropyl methylcellulose, hydroxyethyl cellulose, carboxy methylcellulose, methylcellulose, and mixtures thereof.

In embodiments, the one or more second water-soluble polymers may together be present in the film at about 25% to about 40% w/w of the total film weight when the one or more second water-soluble polymers each has a molecular weight from about 90,000 daltons to about 200,000 daltons and/or from about 200,000 daltons to about 500,000 daltons, and the polymer is selected from the group consisting of hydroxypropyl cellulose, hydroxypropyl methylcellulose, hydroxyethyl cellulose, carboxy methylcellulose, methylcellulose, and mixtures thereof.

In embodiments, polyethylene oxide may be present in the film at about 50% to about 60% w/w of the total film weight.

In embodiments, the polymer component for the film composition consists of a low molecular weight, water-soluble polymer (e.g., having a molecular weight less than about 60,000 daltons) and one or more high molecular weight polymers (e.g., having a molecular weight greater about 60,000, up to about 1,500,000 daltons when a polyethylene oxide is included in the polymer mixture or up to about 500,000 daltons when a polyethylene oxide is not included in the polymer mixture). This polymer combination, especially when the polymers are a combination of hydroxypropyl cellulose and polyethylene oxide, lends certain advantages to the tensile strength and pharmacokinetics of the film composition.

In embodiments, the present disclosure provides a film composition comprising (e.g. consisting essentially of):
(i) a therapeutically effective amount of dexmedetomidine or a pharmaceutically acceptable salt thereof,
(ii) a polymer component consisting of one or more water-soluble polymers: and
(iii) one or more pharmaceutically acceptable carriers.

In embodiments, the present disclosure provides a film composition comprising (e.g. consisting essentially of):
(i) therapeutically effective amount of dexmedetomidine or a pharmaceutically acceptable salt thereof;
(ii) a polymer component consisting of: (a) one or more first water-soluble polymer (e.g. hydroxypropyl cellulose, hydroxypropyl methylcellulose, hydroxyethyl cellulose, carboxy methylcellulose, methylcellulose, and mixtures thereof) having a molecular weight from about 5,000 daltons to about 49,000 daltons, for example, in about 2 to about 15 weight percent on dry weight basis of the total polymer component; and (b) one or more second water-soluble polymers (e.g. polyethylene oxide, hydroxypropyl cellulose, hydroxypropyl methylcellulose, hydroxyethyl cellulose, carboxy methylcellulose, methylcellulose, and mixtures thereof) having a molecular weight greater than 60,000 daltons, such as greater than 100,000 daltons, for example in about 50 to about 98 weight percent on dry weight basis of the total polymer component; and
(iii) one or more pharmaceutically acceptable carriers.

The molecular weight of hydroxypropyl cellulose, when present in the film of the present disclosure, may be varied, and may be present as both a low molecular weight, water-soluble polymer and as one or more high molecular weight, water-soluble polymers. In embodiments, the molecular weight may be less than about 60,000 daltons (e.g. about 5,000 daltons to about 49,000 daltons). In embodiments the molecular weight may be in the range from about 90,000 daltons to about 200,000 daltons. In embodiments, the molecular weight may be in the range from about 200,000 daltons to about 500,000 daltons.

Hydroxypropyl cellulose, when part of the film composition including polyethylene oxide, may conveniently be present in the range from about 10% to about 90% by weight on a dry weight basis of the polymer component, e.g. about 20% to about 80% by weight on dry weight basis of the polymer component, e.g. about 20% to about 50% by weight on dry weight basis of the polymer component, e.g. about 25% to about 45% by weight on dry weight basis of the polymer component.

The molecular weight of polyethylene oxide, when present in the film of the present disclosure, may also be varied. In some embodiments, a water-soluble, high molecular weight polyethylene oxide may be used, for example, to increase muco-adhesivity of the film. In certain embodiments, the molecular weight may range from about 100,000 daltons to about 1,500,000 daltons, including about 100,000, 200,000, 300,000, 600,000, 900,000 or 1,000,000 daltons. In embodiments, it may be desirable to use a combination of polyethylene oxide having a molecular weight of about 600,000 daltons to about 900,000 daltons with polyethylene oxide having a molecular weight of about 100,000 daltons to about 300,000 daltons in the polymer component.

Polyethylene oxide, when part of the film composition, may conveniently be present in range from about 30% to about 90% by weight on a dry weight basis of the total polymer component, e.g. about 40% to about 85% by weight on a dry weight basis of the polymer component, e.g. about 55% to about 80% by weight on a dry weight basis of the polymer component.

Such film compositions may contain the drug dispersed within the film, or micro-deposited onto a surface of the film. When micro-deposited on the surface of a “placebo” film, the drug may conveniently be added as part of a dexmedetomidine composition as one or more droplets in a liquid carrier, such as a solvent (e.g. an alcohol such as ethanol), optionally together with one or more (e.g. two) water-soluble polymers and/or pharmaceutically acceptable carriers. Suitable water-soluble polymers include (1) a low molecular weight, water-soluble polymer, for example a low molecular weight, water-soluble polymer having a molecular weight of less than about 60,000 daltons (e.g. a molecular weight of about 5,000 daltons to about 49,000 daltons and optionally (2) one or more (e.g. one or two) high molecular weight, water-soluble polymers, for example a high molecular weight, water-soluble polymer having a molecular weight of greater than about 60,000 daltons (e.g. a molecular weight of from about 60,000 daltons to about 150,000 daltons such as hydroxypropyl cellulose (77,000 MW), hydroxypropyl cellulose (80,000 MW), hydroxypropyl cellulose (90,000 MW), or hydroxypropyl cellulose (140,000 MW)) and/or a high molecular weight, water-soluble polymer having a molecular weight of greater than about 60,000 daltons (e.g. a molecular weight of from about 200,000 daltons to about 900,000 daltons such as hydroxypropyl cellulose (340,000 MW), hydroxypropyl cellulose (370,000 MW), polyethylene oxide (200,000 MW) or polyethylene oxide (600,000 MW)). Each water-soluble polymer may independently be selected from the group consisting of hydroxypropyl cellulose, hydroxypropyl methylcellulose, hydroxyethyl cellulose, carboxymethyl cellulose, polyethylene oxide and methyl cellulose, e.g. hydroxypropyl cellulose and/or polyethylene oxide.

In embodiments, the dexmedetomidine composition comprises dexmedetomidine hydrochloride, a low molecular weight polymer which is hydroxypropyl cellulose and one or two high molecular weight polymers each of which are hydroxypropyl cellulose in an ethanol solvent.

In embodiments, the dexmedetomidine composition comprises dexmedetomidine or a pharmaceutically acceptable salt thereof (e.g. dexmedetomidine hydrochloride), hydroxypropyl cellulose (40,000 MW) and one or both of hydroxypropyl cellulose (140,000 MW) and hydroxypropyl cellulose (370,000 MW).

In embodiments, the dexmedetomidine composition comprises dexmedetomidine or a pharmaceutically acceptable salt thereof (e.g. dexmedetomidine hydrochloride), and only two hydroxypropyl celluloses, namely hydroxypropyl cellulose (40,000 MW) and hydroxypropyl cellulose (140,000 MW).

In embodiments, the deposition composition may be in any form, including as a solution, emulsion, suspension or dispersion. For example, the dexmedetomidine composition may be added as one or more droplets in an ethanol-based solution, optionally containing a pH-neutralizing agent such as sodium hydroxide. In embodiment, the film substrate surface contains two or more micro-deposited spots of dexmedetomidine hydrochloride (e.g. two micro-deposited spots) in a polymer matrix. The viscosity of deposition solution/suspension may range from about 6 cps to about 3700 cps as measured at 25° C. using a Brookfield viscometer with a small sample adapter. As an example, it may range from about 5 cps to about 500 cps, about 6 cps to about 200 cps, about 6 cps to about 100 cps or about 6 cps to about 50 cps.

In embodiment of the present disclosure, the viscosity of the dexmedetomidine composition is from about 6 cps to about 20 cps at 25° C. and a shear rate of about 7 (1/s).

Following drying to remove the solvent, the film comprises a film substrate (e.g. a placebo) with the dexmedetomidine composition as previously described but absent the solvent deposited (e.g. micro-deposited) on the surface of the film substrate. The dried composition containing dexmedetomidine or a pharmaceutically acceptable salt thereof (e.g. dexmedetomidine hydrochloride) may cover the whole of the film substrate surface or only part of the film substrate surface.

In embodiments, the dried dexmedetomidine composition appears as one or more discrete drug-containing droplets on the film substrate surface. Alternatively, stenciling may be used to achieve a one or more defined and discrete regions of drug-containing composition on the surface of the film substrate.

In embodiments, the disclosure provides a dry film product comprising a film substrate with one or more discrete drug-containing droplets on the film substrate surface, wherein each such drug-containing droplet comprises dexmedetomidine or a pharmaceutically acceptable salt thereof, and hydroxypropyl cellulose of two molecular weights: hydroxypropyl cellulose (40,000 MW) available as HPC-SSL, and hydroxypropyl cellulose (140,000 MW) marketed under the tradename of Klucel™ Type JF NF, and wherein the film substrate comprises hydroxypropyl cellulose of three molecular weights: hydroxypropyl cellulose (40,000 MW), hydroxypropyl cellulose (140,000 MW), and hydroxypropyl cellulose (370,000 MW) marketed under the tradename of Klucel™ Type GF NF. In embodiments, the film substrate also comprises polyethylene oxide (600,000 MW) available under the name of Sentry Polyox WSR 205 LEO NF.

In embodiments, the dry film product comprises a deposition composition (also referred to herein as a “dexmedetomidine composition”) comprising: (i) dexmedetomidine hydrochloride, present at about 9% to about 50% w/w of the deposition composition, e.g. about 15% to about 25% w/w of the deposition composition; (ii) hydroxypropyl cellulose (40,000 MW), present at about 5% to about 85% w/w of the deposition composition; (iii) hydroxypropyl cellulose (140,000 MW) present at about 5% to 85% w/w of the deposition composition; and (iv) hydroxypropyl cellulose (370,000 MW) present at about 0% to about 65% w/w of the deposition composition. The film also comprises a polymer matrix, wherein the polymer matrix comprises: (i) hydroxypropyl cellulose (40,000 MW) present at about 3% to about 40% w/w of the polymer matrix; (ii) hydroxypropyl cellulose (140,000 MW) present at about 3% to about 40% w/w of the polymer matrix; (iii) hydroxypropyl cellulose (370,000 MW) present at about 0% to about 30% w/w of the polymer matrix, and (iv) polyethylene oxide (600,000 MW) present at about 55% to about 75% w/w of the polymer matrix.

In embodiments, the dry film product (e.g. a micro-deposited film product) comprises(i) dexmedetomidine hydrochloride, present at about 1% to about 50% w/w of the total film weight; (ii) hydroxypropyl cellulose (40,000 MW), present at about 2% to about 30% w/w of the total film weight; (iii) hydroxypropyl cellulose (140,000 MW) present at about 2% to about 30% w/w of the total film weight; (iv) hydroxypropyl cellulose (370,000 MW) present at about 10% to about 50% w/w of the total film weight, (v) polyethylene oxide (600,000 MW) present at about 40% to about 75% w/w of the total film weight and (vi) optionally other pharmaceutically acceptable carriers.

In embodiments, the films disclosed herein combine several types of hydroxypropyl cellulose (HPC) to provide a film with advantageous properties. For example, the film composition may contain two or three of hydroxypropyl cellulose (40,000 MW), hydroxypropyl cellulose (140,000 MW) and hydroxypropyl cellulose (370,000 MW) in combination. In certain embodiments, polyethylene oxide (600,000 MW) is included with these types of HPC when part of a monolithic film.

In certain film compositions of the present disclosure, a low molecular weight hydroxypropyl cellulose (e.g. 40,000 MW) is present at about 3% to about 8% (e.g. about 5%) w/w of the total film weight, a high molecular weight hydroxypropyl cellulose (e.g. 140,000 MW) is present at about 3% to about 8% (e.g. about 5%) w/w of the total film weight, a high molecular weight hydroxypropyl cellulose (e.g. 370,000 MW) is present at about 20% to about 40% w/w of the total film weight, and polyethylene oxide (e.g. 600,000 MW) is present at about 40% to about 70%, (e.g. about 50% to about 60%) w/w of the total film weight. In embodiments, the two high molecular weight, water-soluble polymers are together present at about 25% to about 40% w/w of the total film weight.

The selection and ratio of water-soluble polymers can be made to effect complete dissolution of the film composition in oral mucosal fluids within seconds to minutes, e.g. in about 0.25 minutes to about 15 minutes, thus ensuring delivery of a therapeutically effective amount of dexmedetomidine or a pharmaceutically acceptable salt thereof via the oral mucosa. For example, the film compositions may reside in the sublingual or buccal region of the mouth up to about 15 minutes, up to about 10 minutes, or up to about 5 minutes, including for a period of from about 30 seconds to about 15 minutes, about 1 minute to about 10 minutes, or about 1 minute to about 5 minutes.

The standard basket or paddle apparatus described in any pharmacopoeia can be used for in vitro dissolution testing. The selection of dissolution medium will essentially depend as per the sink conditions and highest dose of drug. The temperature of dissolution medium should be maintained at 37±0.5° C. and rpm at 50 (see Bala et al., in Int J Pharm Investigation, vol. 3(2), pages 67-76).

Films disclosed herein have several functional advantages to promote rapid onset of drug effect. In embodiments, thin films compositions of the disclosure have a disintegration time (DT) of about 15 seconds to about 180 seconds, about 15 seconds to about 160 seconds, about 25 seconds to about 150 seconds, about 15 seconds to about 140 seconds, about 15 seconds to about 120 seconds, about 40 seconds to about 120 seconds, about 50 seconds to about 120 seconds, for example about 120 seconds, when applied oromucosally (e.g. sublingually or buccally). A disintegration time in this time-frame provides optimal onset of drug effects.

In embodiments, thin film compositions of the disclosure have mucoadhesion properties that provide practical benefits of localizing the film to the sublingual location and reducing, or preventing, effective removal prior to dissolution. This quality is particularly advantageous in a clinical setting with an agitated subject. Thus, in embodiments, thin film compositions have a mucoadhesion force (the mucoadhesion strength or shear strength) of about 50 g or above, about 100 g or above, about 200 g or above, about 300 g or above, about 400 g or above, about 500 g or above, about 600 g or above, about 700 g or above, about 800 g or above, about 900 g or above, about 1000 g or above. In embodiments, the mucoadhesion force is in a range of about 300 g to about 4000 g, about 500 g to about 3000 g, or about 1000 g to about 2000 g.

Burst strength of the film also contributes to drug delivery. Certain thin film compositions of the disclosure have a burst strength at or above 50 g, 100 g, 200 g, 300 g, 400 g, 500 g, 600 g, 700 g, 800 g, 900 g, 1000 g, 1100 g, 1200 g, 1300 g, 1400 g, 1500 g, 1600 g, 1700 g, 1800 g, 1900 g, 2,000 g, 2,500 g, 3,000 g, 3,500 g, 4,000 g, 4,500 g, 5,000 g, 5,500 g, 6,000 g, 6,500 g, 7,000 g, 7,500 g, 8,000 g, 8,500 g, 9,000 g, 9,500 g, 10,000 g or 15,000 g. For example, the burst strength may be in a range of about 200 g to about 15000 g, about 300 g to about 10,000 g, or about 400 g to about 5,000 g.

Pharmaceutically Acceptable Carriers

The film compositions may further comprise one or more pharmaceutically acceptable carriers that includes, but is not limited to, liquid carriers, flavours, sweeteners, refreshing agents, antioxidants, pH adjusting agents, permeation enhancers, mucoadhesive agents, plasticizers, bulking agents, surfactants/non-ionic solubilizers, stabilizers, anti-foam agents, colors or the like. In certain embodiments, the film compositions are substantially free of acidic buffer or other acidic agents.

Liquid Carriers

According to embodiments, the pharmaceutically acceptable carrier includes a liquid carrier. The liquid carrier comprises one or more solvents useful in the preparation of the polymer matrix (drug containing or placebo) and deposition composition on the polymer matrix. In some embodiments, the solvent may be water. In embodiments, the solvent may a polar organic solvent including, but are not limited to, ethanol, isopropanol, acetone, butanol, benzyl alcohol and mixtures thereof. In embodiments, the solvent may be a non-polar organic solvent, such as methylene chloride, toluene, ethyl acetate and mixtures thereof. Certain solvents are alcohols, especially ethanol, water and mixtures thereof. Desirably, the solvent content in the wet polymer matrix is at least about 30% by weight of the total wet weight of the total film composition prior to drying. The subsequent dried film composition will desirably contain less than about 10% by weight of solvent, more desirably less than about 8% by weight of solvent, even more desirably less than about 6% by weight of solvent and most desirably less than about 2% by weight of solvent.

Flavors/Sweeteners/Refreshing Agents

It may be beneficial to add a sweetener, flavoring agent, refreshing agent, taste-masking agent or a combination thereof to the film compositions to improve the film composition taste. Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. Non-limiting flavor oils include: spearmint oil, cinnamon oil, peppermint oil, clove oil, bay oil, thyme oil, cedar leaf oil, oil of nutmeg, oil of sage, and oil of bitter almonds. In embodiments, the flavor is a peppermint oil flavour available as peppermint oil, NF.

The amount may be varied in order to obtain the result desired in the final product. Such variations are within the capabilities of those skilled in the art without the need for undue experimentation. In general, amounts of about 0.1% to about 30 wt % may be used in the films to supply flavoring. Suitable sweeteners include both natural and artificial sweeteners. Non-limiting examples of suitable sweeteners include, e.g.: water-soluble sweetening agents such as monosaccharides, disaccharides and polysaccharides such as xylose, ribose, glucose (dextrose), mannose, galactose, fructose (levulose), sucrose (sugar), high fructose corn syrup, maltose, invert sugar (a mixture of fructose and glucose derived from sucrose), partially hydrolyzed starch, corn syrup solids, and dihydrochalcones; water-soluble artificial sweeteners such as the soluble saccharin salts, i.e., sodium or calcium saccharin salts, cyclamate salts and water-soluble sweeteners derived from naturally occurring water-soluble sweeteners, such as a chlorinated derivatives of ordinary sugar (sucrose), known, for example, as sucralose. In one embodiment, the sweetener is sucralose.

Flavoring agents, sweeteners and refreshing agents can be added in conventional quantities, generally up to a total amount of about 0.01% to about 10% of the weight of the film on a dry weight basis, e.g. from about 0.1% to about 7% of the weight of the film on a dry weight basis, e.g. about 0.1% to about 5% based on the weight of the film on a dry weight basis.

Other taste-masking agents include, for example polymers, oils, or waxes. In one embodiment, dexmedetomidine or a pharmaceutically acceptable salt thereof is coated with a taste-masking agent prior to formulation of the film compositions. In embodiments, if a taste-masking agent is used to coat the active ingredient, it may be present in an amount of from about 5% to about 80% by weight of the particle or granule containing the active ingredient. In embodiment, the taste-masking agent is present in an amount from about 25% to about 35% by weight of the particle or granule containing the active ingredient.

Antioxidants

Examples of oxygen scavengers or antioxidants that substantially improve long-term stability of the film composition against oxidative degradation include sulfite salts, such as sodium sulfite, sodium bisulfite, sodium metabisulfite and analogous salts of potassium and calcium. A suitable amount of the sulfite salt (e.g., sodium sulfite) is up to about 5%, e.g. about 0.001% to about 2% based on the weight of the film composition on a dry weight basis.

pH-Adjusting Agents/pH-Neutralizing Agents

The absorption of dexmedetomidine or a pharmaceutical acceptable salt thereof through the oral mucosa may increase in an alkaline microenvironment. As an example, this may be achieved when the film compositions are maintained at a pH of above 6, from about 6 to about 9, or about 6.5 to about 8. In embodiments, the film may include an alkaline substance that increases the pH of the film product. Non-limiting examples of pH-adjusting/pH-neutralizing agents include bicarbonates (e.g., sodium bicarbonate), citrates (e.g., potassium citrate), carbonates (e.g., calcium carbonate), lactates (e.g., sodium lactate), acetates (e.g., calcium acetate), alkaline buffer (e.g. glycine), sodium hydroxide, sodium chloride or the like. An alkaline buffer, such as glycine, is one example of a pH-neutralizing agent. A suitable amount of pH-adjusting/pH-neutralizing agent present in the film composition includes, for example, up to about 10%, e.g. about 1% to about 5% based on the weight of the film composition on a dry weight basis

Permeation Enhancer Agents

Certain effective penetration enhancers that promote absorption of dexmedetomidine or a pharmaceutically acceptable salt thereof across the oral mucosa include alcohols. An alcohol penetration enhancer, such as butanol, can conveniently be added to the film composition in an amount of up to about 10%, e.g. about 0.1% to about 5%, e.g. about 1% to about 3% based on the weight of the film composition on a dry weight basis.

Mucoadhesive Agents

Examples of mucoadhesive agents that can be added to the film composition include, but are not limited to, sodium alginate, sodium carboxymethyl cellulose, guar gum, polyethylene oxide, hydroxyethyl cellulose, hydroxypropyl cellulose, hydroxypropylmethyl cellulose, karaya gum, methylcellulose, retene, tragacanth and the like. One mucoadhesive agent is polyethylene oxide, which may conveniently be added to the film composition in an amount of from about 20% to about 90%, e.g. about 40% to about 70% based on the total weight of the film composition on a dry weight basis.

Plasticizers

Plasticizers that can be effectively employed herein include polyethylene glycol, propylene glycol, tributyl citrate, triethyl citrate and glycerol. Depending on the selected film-forming polymer(s) and other components of the film formulation, a suitable amount of plasticizer included in the film composition may typically be up to about 10%, e.g. about 0.1% to about 5%, e.g. about 0.5% to about 5% based on the weight of the film on a dry weight basis. For certain applications, higher molecular weight polyethylene glycols may be utilized, including polyethylene oxide.

Fillers:

Suitable fillers that can be added to a film composition include starch, calcium salts, such as calcium carbonate, and sugars, such as lactose, glucose, sucrose, mannose, sorbitol, mannitol, galactitol, sucralose, trehalose and combinations thereof. The amount of filler that can conveniently be added to the film formulation is typically up to about 25%, e.g. about 0.5% to about 20%, e.g. about 1% to about 15%, e.g. about 2% to about 10%, based on the weight of the film composition on a dry weight basis.

Surfactants/Non-Ionic Solubilizers

The film typically incorporates at least one surfactant/non-ionic solubilizer including, for example, but are not limited to, a poloxamer, polyoxyl hydrogenated castor oil, glyceryl polyethylene glycol oxystearates, fatty acid glyceryl polyglyceryl esters, polyglyceryl esters, and combinations thereof. The amount of surfactant(s) that can be added to the film composition is typically up to about 5%, e.g. about 0.5% to about 3%, e.g. about 1% to about 3% based on the weight of the film composition on a dry weight basis.

Anti-Foaming Components

Simethicone is an example of a useful anti-foaming and/or de-foaming agent, although other anti-foaming and/or de-foaming agents may suitable be used. An anti-foaming and/or de-foaming agent such as simethicone may be added to the film composition in an amount from about 0.01% to about 5.0%, more desirably from about 0.05% to about 2.5%, and most desirably from about 0.1% to about 1.0% based on the weight of the film composition on a dry weight basis.

Colorants

Color additives that may be included in a film composition include food, drug and cosmetic colors (FD&C), drug and cosmetic colors (D&C), or external drug and cosmetic colors (Ext. D&C). These colors are dyes, their corresponding lakes, and certain natural and derived colorants. Certain examples of color additives are inorganic pigments, such as oxides of iron or titanium, added in concentrations ranging from about 0.001% to about 10%, e.g. about 0.01% to about 3%, based on the weight of the film composition on a dry weight basis. In embodiments, the color used for the dexmedetomidine composition (i.e. the deposit composition) is different from the color used for the film substrate (e.g. the placebo film). One color of the monolithic film and the film substrate of the micro-deposited film is emerald green, and available as Fast Emerald Green Shade (06507). One color of the dexmedetomidine composition (i.e. the deposit composition) is a different from the color of the film substrate, e.g. blue (available as FD&C Blue No. 1). In embodiments of the film embodiments of the present disclosure, for example, as described in aspects and embodiments hereinabove, is a film comprising about 180 μg of dexmedetomidine or a pharmaceutically acceptable salt thereof containing two blue color micro-deposited spots of dexmedetomidine hydrochloride on the green color film substrate.

In embodiments of the film embodiments of the present disclosure, for example, as described in aspects and embodiments hereinabove, is a film comprising about 120 μg of dexmedetomidine or a pharmaceutically acceptable salt thereof.

In embodiment (A), there is provided a self-supporting, dissolvable, film, comprising:
(i) about 180 μg of dexmedetomidine or a pharmaceutically acceptable salt thereof (e.g. the hydrochloride salt);
(ii) one or more water-soluble polymers;
(iii) polyethylene oxide and, optionally,
(iv) one or more pharmaceutically acceptable carriers.

In embodiment (B), there is provided a self-supporting, dissolvable, film, comprising:
(i) about 120 μg of dexmedetomidine or a pharmaceutically acceptable salt thereof (e.g. the hydrochloride salt);
(ii) one or more water-soluble polymers;
(iii) polyethylene oxide and, optionally,
(iv) one or more pharmaceutically acceptable carriers.

In embodiments, the just-mentioned one or more water-soluble polymers (ii) of embodiment (A) or (B) above comprises a low molecular weight, water-soluble polymer and two high molecular weight, water-soluble polymers, for example wherein the low molecular weight, water-soluble polymer has a molecular weight from about 5,000 daltons to about 49,000 daltons (e.g. about 40,000 daltons), and each high molecular weight, water-soluble polymer has a molecular weight of greater than about 60,000 daltons (e.g. where one of the two high molecular weight, water-soluble polymers has a molecular weight of about 140,000 daltons, and the other high molecular weight, water-soluble polymer has a molecular weight of about 370,000 daltons). Each water-soluble polymer is, in some embodiments, hydroxypropyl cellulose. The polyethylene oxide, in some embodiments, has a molecular weight of about 600,000 daltons.

In embodiments, there is provided a pharmaceutical film composition comprising or consisting essentially of therapeutically effective amount of dexmedetomidine or pharmaceutically acceptable salt thereof and one or more excipients selected from polyethylene oxide, hydroxypropyl cellulose, sucralose, peppermint oil, emerald green colorant, and FD&C blue colorant.

In embodiment (C), there is provided a self-supporting, dissolvable, film, comprising:
(i) about 180 μg of dexmedetomidine or a pharmaceutically acceptable salt thereof (e.g. the hydrochloride salt);
(ii) a low molecular weight, water-soluble polymer having a molecular weight of about 40,000 daltons;
(iii) a high molecular weight, water-soluble polymer having a molecular weight from about 140,000 daltons;
(iv) a high molecular weight, water-soluble polymer having a molecular weight from about 370,000 daltons; and
(v) a water-soluble polyethylene oxide having a molecular weight of about 600,000 daltons.

In embodiment (D), there is provided a self-supporting, dissolvable, film, comprising:
(i) about 120 μg of dexmedetomidine or a pharmaceutically acceptable salt thereof (e.g. the hydrochloride salt);
(ii) a low molecular weight, water-soluble polymer having a molecular weight of about 40,000 daltons;
(iii) a high molecular weight, water-soluble polymer having a molecular weight from about 140,000 daltons;
(iv) a high molecular weight, water-soluble polymer having a molecular weight from about 370,000 daltons; and
(v) a water-soluble polyethylene oxide having a molecular weight of about 600,000 daltons.

In embodiment of the just-mentioned films of embodiments (C) and (D), the film components excluding dexmedetomidine or a pharmaceutically acceptable salt thereof form a single layer film substrate, and dexmedetomidine or a pharmaceutically acceptable salt thereof is present on the surface of the film substrate (e.g. within a composition comprising dexmedetomidine or a pharmaceutically acceptable salt thereof, a low molecular weight, water-soluble polymer having a molecular weight of about 40,000 daltons, and a high molecular weight, water-soluble polymer having a molecular weight of about 140,000 daltons). Each water-soluble polymer is, in some embodiments, hydroxypropyl cellulose.

In embodiment (E), there is provided a self-supporting, dissolvable, film, comprising:
(a) a composition consisting essentially of:
   (i) about 180 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);
      wherein the composition of part (a) is present on the surface of the film substrate (b).

In embodiment (F), there is provided a self-supporting, dissolvable, film, comprising:
(a) a composition consisting essentially of:
   (i) about 120 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);
      wherein the composition of part (a) is present on the surface of the film substrate (b).

In embodiment of the just-mentioned films of embodiments (E) and (F), dexmedetomidine hydrochloride is present at about 0.1% to about 2% w/w of the total film weight, hydroxypropyl cellulose (40,000 MW) is present at about 4% to about 8% w/w of the total film weight, hydroxypropyl cellulose (140,000 MW) is present at about 4% to about 8% w/w of the total film weight, hydroxypropyl cellulose (370,000 MW) is present at about 25% to about 30% w/w of the total film weight, and polyethylene oxide (600,000 MW) is present at about 50% to about 60% w/w of the total film weight.

In embodiments, the present disclosure provides pharmaceutical buccal film compositions comprising or consisting essentially of therapeutically effective amount of dexmedetomidine or pharmaceutically acceptable salt thereof, one or more mucoadhesive polymers and optional excipients selected from one or more of plasticizers, penetration enhancers, coloring agents, sweetening agents, flavoring agents, taste-making agents or salivary stimulants. Mucoadhesive polymers may be selected from hydrophilic polymers and hydrogels. Examples of hydrophilic polymers include polyvinyl alcohol [PVA], sodium carboxy methylcellulose [NaCMC], hydroxyl propyl methyl cellulose [HPMC], hydroxyl ethyl cellulose and hydroxypropyl cellulose [HPC]. Examples of hydrogels include anionic polymers like carbopol, polyacrylates, cationic polymers like chitosan and non-ionic polymers like Eudragit analogues.

Sprays, Drops or Gels

In embodiments, the present disclosure provides pharmaceutical spray compositions or drop compositions suitable for oromucosal (e.g. sublingual or buccal) administration comprising or consisting essentially of a therapeutically effective amount of dexmedetomidine or pharmaceutically acceptable salt thereof and one or more pharmaceutically acceptable liquids (from about 1% to about 99.995% by weight). Such liquids may be solvents, co-solvents, or non-solvents for dexmedetomidine or a pharmaceutically acceptable salt thereof. Examples of pharmaceutically acceptable liquids include water, ethanol, dimethyl sulfoxide, propylene glycol, polyethylene glycol, propylene carbonate, glycerine, N-methylpyrrolidone, pharmaceutically acceptable oils (e.g., soybean, sunflower, peanut, etc.) or the like. The pharmaceutically acceptable liquid is selected either to dissolve dexmedetomidine or pharmaceutically acceptable salt thereof, to produce a stable, homogenous suspension of it, or to form any combination of a suspension or solution. In addition to these ingredients, spray or drop formulations of dexmedetomidine or pharmaceutically acceptable salt thereof may include one or more excipients such as viscosity modulating materials (e.g. polymers, sugars, sugar alcohols, gums, clays, silicas, and the like, such as polyvinylpyrrolidone (PVP)); preservatives (e.g., ethanol, benzyl alcohol, propylparaben and methylparaben); flavoring agents (e.g. peppermint oil), sweeteners (e.g., sugars such as sucrose, glucose, dextrose, maltose, fructose, etc.), artificial sweeteners (e.g. saccharin, aspartame, acesulfame, sucralose), or sugar alcohols (e.g. mannitol, xylitol, lactitol, maltitol syrup); buffers and pH-adjusting agent (e.g., sodium hydroxide, citrate, and citric acid); coloring agents; fragrances, chelating agents (e.g., EDTA); UV absorbers and antifoam agents (e.g., low molecular weight alcohols, dimethicone). In addition to one or more of the aforementioned ingredients suitable for oromucosal (e.g. sublingual or buccal) sprays or drops, oromucosal gel formulations of dexmedetomidine or pharmaceutically acceptable salt thereof may include one or more excipients such as viscosity modulating materials (e.g. water soluble or water swellable polymers such as carbopol, hydroxypropyl cellulose, hydroxypropyl methyl cellulose, carboxymethyl cellulose).

Sprays, drops, and gels may be made by mixing appropriate quantities of the foregoing ingredients in accordance with standard good manufacturing practices. Such excipients may be included in the formulation to improve patient or subject acceptance or taste, to improve bioavailability, to increase shelf-life, to reduce manufacturing and packaging costs, to comply with requirements of governmental regulatory agencies, and for other purposes. The relative amounts of each ingredient should not interfere with the desirable pharmacological and pharmacokinetic properties of the resulting formulation.

In embodiments, there is provided an oromucosal spray composition comprising or consisting essentially of therapeutically effective amount of dexmedetomidine or pharmaceutically acceptable salt thereof and one or more pharmaceutically acceptable carrier or excipients.

A patient may, in one embodiment, be treated by administering sublingually or buccally 1 to 2 actuations from a spray pump. An advantage of spray delivery is the ability to easily titrate patients by 1 or 2 doses as required by a single actuation.

Pump action sprays are characterized in requiring the application of external pressure for actuation, for example, external manual, mechanical or electrically initiated pressure. This is in contrast to pressurized systems, e.g., propellant-driven aerosol sprays, where actuation is typically achieved by controlled release of pressure e.g., by controlled opening of a valve.

Various sublingual spray formulations comprising dexmedetomidine hydrochloride at doses of 20 μg, 30 μg, 60 μg, 90 μg, 120 μg and 180 μg and excipients as described in table 1.

TABLE 1
Sublingual spray formulation embodiments
according to the disclosure

| | Sublingual Spray Formulation Embodiment No. | | | |
|---|---|---|---|---|
| Ingredients | 1 | 2 | 3 | 4 |
| N-methylpyrrolidone | ✓ | | | |
| Propylene Glycol | | ✓ | | |
| Polyethylene Glycol | | | ✓ | |
| Glycerine | | | | ✓ |
| Ethanol | ✓ | ✓ | ✓ | ✓ |
| Sucralose | ✓ | ✓ | ✓ | ✓ |
| Peppermint Oil | ✓ | ✓ | ✓ | ✓ |
| Purified water | ✓ | ✓ | ✓ | ✓ |
| Optionally other | ✓ | ✓ | ✓ | ✓ |
| pharmaceutically | | | | |
| acceptable excipients | | | | |

Various sublingual drop compositions comprising dexmedetomidine hydrochloride at doses of 20 μg, 30 μg, 60 μg, 90 μg, 120 μg and 180 μg and excipients as described in table 2.

TABLE 2
Sublingual drop formulations embodiments according to the disclosure

| | Sublingual Drop Formulation Embodiment No. | | | | | | | | | | | | | |
|---|---|---|---|---|---|---|---|---|---|---|---|---|---|---|
| Ingredients | 1 | 2 | 3 | 4 | 5 | 6 | 7 | 8 | 9 | 10 | 11 | 12 | 13 | 14 |
| Povidone | ✓ | ✓ | ✓ | ✓ | ✓ | | | | | | | | | |
| N-methylpyrrolidone | ✓ | | | | | ✓ | | | | ✓ | | | | |
| Hydroxypropyl | | | | | | ✓ | ✓ | ✓ | ✓ | | | | | |
| methylcellulose | | | | | | | | | | | | | | |
| Carbopol | | | | | | | | | | ✓ | ✓ | ✓ | ✓ | ✓ |
| Polyethylene glycol | | | ✓ | | | | | | | | | ✓ | | |
| Propylene glycol | | ✓ | | | | | ✓ | | | | ✓ | | | |
| Glycerine | | | | ✓ | | | | | ✓ | | | | ✓ | |
| Ethanol | | | | | ✓ | | | ✓ | | | | | | ✓ |
| Sucralose | ✓ | ✓ | ✓ | ✓ | ✓ | ✓ | ✓ | ✓ | ✓ | ✓ | ✓ | ✓ | ✓ | ✓ |
| Peppermint Oil | ✓ | ✓ | ✓ | ✓ | ✓ | ✓ | ✓ | ✓ | ✓ | ✓ | ✓ | ✓ | ✓ | ✓ |
| Purified water | ✓ | ✓ | ✓ | ✓ | ✓ | ✓ | ✓ | ✓ | ✓ | ✓ | ✓ | ✓ | ✓ | ✓ |
| Optionally other | ✓ | ✓ | ✓ | ✓ | ✓ | ✓ | ✓ | ✓ | ✓ | ✓ | ✓ | ✓ | ✓ | ✓ |
| pharmaceutically | | | | | | | | | | | | | | |
| acceptable excipients | | | | | | | | | | | | | | |

Various sublingual gel compositions comprising dexmedetomidine hydrochloride at doses of 20 μg, 30 μg, 60 μg, 90 μg, 120 μg and 180 μg and excipients as described in table 3.

TABLE 3
Sublingual gel formulations embodiments according to the disclosure.

| | Sublingual Gel Formulation Embodiment Nos. | | | | | | | | | | | | | | |
|---|---|---|---|---|---|---|---|---|---|---|---|---|---|---|---|
| Ingredients | 1 | 2 | 3 | 4 | 5 | 6 | 7 | 8 | 9 | 10 | 11 | 12 | 13 | 14 | 15 |
| Carbopol | ✓ | | | ✓ | | | ✓ | | ✓ | | | | ✓ | | |
| Hydroxypropyl | | ✓ | | | ✓ | | | ✓ | | | ✓ | | | ✓ | |
| methylcellulose | | | | | | | | | | | | | | | |
| Hydroxypropyl cellulose | | | | | | | | | | | | | | | |
| Carboxymethyl cellulose | | | ✓ | | | ✓ | | | | ✓ | | ✓ | | | ✓ |
| N-Methylpyrrolidone | | | | ✓ | ✓ | ✓ | | | | | | | | | |
| Propylene glycol | | | | | | | ✓ | ✓ | | ✓ | | | | | |
| Polyethylene glycol | | | | | | | | | ✓ | | ✓ | ✓ | | | |
| Glycerine | | | | | | | | | | | | | ✓ | ✓ | ✓ |
| Ethanol | ✓ | ✓ | ✓ | | | | | | | | | | | | |
| Sucralose | ✓ | ✓ | ✓ | ✓ | ✓ | ✓ | ✓ | ✓ | ✓ | ✓ | ✓ | | ✓ | ✓ | ✓ |
| Peppermint oil | | ✓ | ✓ | ✓ | ✓ | ✓ | ✓ | ✓ | ✓ | ✓ | ✓ | | ✓ | ✓ | ✓ |
| Purified water | ✓ | ✓ | ✓ | ✓ | ✓ | ✓ | ✓ | ✓ | ✓ | ✓ | ✓ | ✓ | ✓ | ✓ | ✓ |
| Optionally other | ✓ | ✓ | ✓ | ✓ | ✓ | ✓ | ✓ | ✓ | ✓ | ✓ | ✓ | ✓ | ✓ | ✓ | ✓ |
| pharmaceutically | | | | | | | | | | | | | | | |
| acceptable excipients | | | | | | | | | | | | | | | |

Tablets

In embodiments, the present disclosure provides tablet formulations suitable for oromucosal administration (e.g. sublingual or buccal administration) comprising or consisting essentially of therapeutically effective amount of dexmedetomidine or pharmaceutically acceptable salt thereof and one or more pharmaceutically acceptable carrier (from about 1% to about 99.995% by weight). Such carriers may be taste masking agents, diluents, disintegrants, binders, lubricants, glidants, flavouring agents or liquid solvents. Examples of pharmaceutically acceptable liquids include water, ethanol, dimethyl sulfoxide, propylene glycol, polyethylene glycol, propylene carbonate, glycerine, N-methylpyrrolidone, pharmaceutically acceptable oils (e.g., soybean, sunflower, peanut, etc.) or the like. Taste masking agents include, for example, amberlite, Opadry® AMB TAN, polymethacrylates (especially Eudragit© L100), sodium starch glycolate (Primojel), carbopol polymers, PEG-5M, sodium acetate, ethylcellulose, betacyclodextrin. Flavouring agents may be, for example, mint powder, menthol, vanillin, aspartame, acesulfame potassium, saccharin. Disintegrants include, for example, sodium starch glycolate, low-substituted hydroxy propyl cellulose, alginic acid, carbon dioxide, carboxymethylcellulose calcium, carboxymethylcellulose sodium, croscarmellose sodium, guar gum, methylcellulose, polacrilin potassium, poloxamer, sodium alginate. Diluents may be, for example, microcrystalline cellulose, dextrates, dextrose, fructose, mannitol, sucralose, sorbitol, starch, pregelatinized starch, sucrose, xylitol, maltose, maltodextrin, maltitol. Binders may be, for example, alginic acid, carbomer, ethyl cellulose, gelatine, liquid glucose, guar gum, hydroxyethyl cellulose, methylcellulose, polydextrose, polyethylene oxide, hydroxypropyl methylcellulose, hydroxypropyl cellulose, sodium alginate. At least one lubricant may conveniently be incorporated into the formulation to prevent the powder from adhering to tablet punches during the compression procedure. Lubricants may be, for example, talc, magnesium stearate, calcium stearate, glyceryl behenate, hydrogenated castor oil, stearic acid, sodium lauryl sulphate. Glidants are used to promote powder flow by reducing interparticle friction and cohesion. These are used in combination with lubricants as they have no ability to reduce die wall friction. Glidants, may be, for example, colloidal silicon dioxide, calcium silicate, calcium phosphate tribasic.

Various buccal tablet formulations comprising dexmedetomidine hydrochloride at doses of 20 μg, 30 μg, 60 μg, 90 μg, 120 μg and 180 μg and excipients as described in table 4.

TABLE 4
Buccal tablet formulation embodiments according to the disclosure.

| | Buccal Tablet Formulation Embodiment No. | | | | |
|---|---|---|---|---|---|
| Ingredients | 1 | 2 | 3 | 4 | 5 |
| Lactose monohydrate | ✓ | ✓ | ✓ | ✓ | ✓ |
| Polyethylene oxide | ✓ | | | | |
| Hydroxypropyl cellulose | | ✓ | | | |
| Hydroxypropyl | | | | | ✓ |
| methylcellulose | | | | | |
| Sodium alginate | | | | ✓ | |
| Xanthan gum | | | ✓ | | |
| Sucralose | ✓ | ✓ | ✓ | ✓ | ✓ |
| Magnesium stearate | ✓ | ✓ | ✓ | ✓ | ✓ |
| Talc | | ✓ | ✓ | ✓ | ✓ |
| Optionally other | ✓ | ✓ | ✓ | ✓ | ✓ |
| pharmaceutically | | | | | |
| acceptable excipients | | | | | |

Various sublingual tablet compositions comprising dexmedetomidine hydrochloride at doses of 20 μg, 30 μg, 60 μg, 90 μg, 120 g and 180 μg and excipients as described in table 5.

TABLE 5
Sublingual tablet formulation embodiments according to the disclosure.

| | Sublingual Tablet Formulation Embodiment No. | | | | | | | | | |
|---|---|---|---|---|---|---|---|---|---|---|
| Ingredients | 1 | 2 | 3 | 4 | 5 | 6 | 7 | 8 | 9 | 10 |
| Lactose Monohydrate | ✓ | ✓ | ✓ | ✓ | ✓ | ✓ | ✓ | ✓ | ✓ | ✓ |
| Hydroxypropyl | ✓ | ✓ | | | | | | | | |
| methylcellulose | | | | | | | | | | |
| Hydroxypropyl cellulose | | | ✓ | ✓ | | | | | | |
| Croscarmellose Sodium | ✓ | | ✓ | | ✓ | | ✓ | | ✓ | |
| Sodium starch glycolate | | ✓ | | ✓ | | ✓ | | ✓ | | ✓ |
| Polyethylene oxide | | | | | ✓ | ✓ | | | | |
| Xanthan gum | | | | | | | ✓ | ✓ | | |
| Sodium alginate | | | | | | | | | ✓ | ✓ |
| Sucralose | ✓ | ✓ | ✓ | ✓ | ✓ | ✓ | ✓ | ✓ | ✓ | ✓ |
| Magnesium stearate | ✓ | ✓ | ✓ | ✓ | ✓ | ✓ | ✓ | ✓ | ✓ | ✓ |
| Optionally other | ✓ | ✓ | ✓ | ✓ | ✓ | ✓ | ✓ | ✓ | ✓ | ✓ |
| pharmaceutically | | | | | | | | | | |
| acceptable excipients | | | | | | | | | | |

Parenteral Formulations:

Liquid pharmaceutical compositions for parenteral administration may be formulated for administration by injection or continuous infusion. Routes of administration by injection or infusion can include, but are not limited to, intravenous, intraperitoneal, intramuscular, intrathecal, and subcutaneous. In embodiments, parenteral formulations can include prefilled syringes, vials, powder for infusion for reconstitution, concentrate for infusion to be diluted before delivery (ready to dilute) or solutions (ready to use).

Injectable pharmaceutical compositions can be aqueous isotonic solutions or suspensions, and suppositories can be prepared from fatty emulsions or suspensions.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Injectable formulation may further contain liquid vehicles (oily or aqueous), suspending agent, stabilizing and/or dispersing agents, solubilizing agents or solubilizers or surfactants, preservative, pH adjusters, tonicity adjusters or the like. Alternatively, dry powder form injection or sterile solid lyophilized powder(s) of the active ingredient(s) with suitable vehicle, such as sterile, pyrogen-free water may also be used for injection compositions. The parenteral compositions may be supplied in various delivery forms, e.g. ampoules, pre-filled syringes, needle or needle free auto-injectors, as a small volume infusion or in multi-dose containers with an added preservative.

In embodiments, the pharmaceutical compositions of the present disclosure include biodegradable subcutaneous implant, osmotically controlled device, subcutaneous implant, subcutaneous sustained release injection, lipid nanoparticles, liposomes, and the like. Liquid preparations can include, but are not limited to, solutions, suspensions and emulsions. Such preparations are exemplified by water or water/propylene glycol solutions for parenteral injection. Liquid preparations may also include solutions for intranasal administration.

For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled to render the preparation isotonic.

The liquid vehicle used for the preparation of the intramuscular injection may be, for example, water, a saline solution, another aqueous liquid (aqueous solvent) or non-aqueous liquid (non-aqueous solvent).

The parenteral formulations of the present disclosure can be sterilized. Non-limiting examples of sterilization techniques include filtration through a bacterial-retaining filter, terminal sterilization, incorporation of sterilizing agents, irradiation, and heating.

Administration of the above-described parenteral formulations may be by periodic injections of a bolus of the preparation, or may be administered by intravenous or intraperitoneal administration from a reservoir which is external (e.g., an intravenous bag) or internal (e.g., a bioerodable implant, a bioartificial or organ). See, e.g., U.S. Pat. Nos. 4,407,957 and 5,798,113, each incorporated herein by reference in their entireties. Intrapulmonary delivery methods and apparatus are described, for example, in U.S. Pat. Nos. 5,654,007, 5,780,014, and 5,814,607, each incorporated herein by reference in their entireties. Other useful parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aerosolizer, electroporation, and transdermal patch. Needle-less injector devices are described in U.S. Pat. Nos. 5,879,327; 5,520,639; 5,846,233 and 5,704,911, the specifications of which are herein incorporated herein by reference in their entireties. Any of the formulations described herein can be administered in these methods. Further injectable formulations of dexmedetomidine are disclosed in U.S. Pat. Nos. 8,242,158, 9,649,296, JP. Patent No. 5,921, 928, JP. Pat. Appl. No. 2016154598, CN Pat. Appl. No. 103284945, CN Pat. Appl. No. 104161760, CN Pat. Appl. No. 105168122, CN Pat. Appl. No. 105534891, CN Pat. Appl. No. 106038538, U.S. Pat. Appl. No. 20170128421, CN Pat. Appl. No. 107028880, CN Pat. Appl. No. 107412152, CN Pat. Appl. No. 108498469, EP Patent. No. 2252290, JP. Pat. Appl. No. 2019048091 and U.S. Pat. Appl. No. 20190183729.

In certain non-limiting embodiments, the intramuscular composition of the present disclosure comprises dexmedetomidine, or a pharmaceutically acceptable salt thereof, at a concentration of between about 0.05 μg/mL and about 15 μg/mL, sodium chloride at a concentration of between about 0.01 and about 2.0 weight percent and pH in the range of about 1 to about 10.

The intramuscular compositions of the present disclosure can be manufactured by the skilled person by use of standard methods and conventional techniques appropriate to the desired formulation. The formulation for intramuscular administration of the present disclosure can be packed and/or stored in a suitable container, including, without limitation, syringes, ampoules, vials, including sealed vials such as vials the openings of which are sealed with syringe pierceable septa or sure-seals caps, and the like. In embodiment, the formulation is pre-filled in disposable syringes for self-administration by patients, with or without an auto-injector.

Oral Formulations:

The present disclosure includes oral formulations that can be used for delivering dexmedetomidine. Examples of oral formulations includes tablets, orally disintegrating tablets, mouth dissolving tablets, wafers, solution, suspension, emulsions, and capsules.

The disclosure encompasses oral disintegrating tablets comprising dexmedetomidine or a pharmaceutically acceptable salt thereof and at least one orally disintegrating carrier, wherein the oral disintegrating tablet disintegrates in about 0.5 to about 120 seconds and/or a therapeutically effective amount of the dexmedetomidine is absorbed into the bloodstream within about 1 to about 5 minutes. In embodiments, a therapeutically effective amount of the dexmedetomidine is absorbed into the bloodstream within about 3 minutes.

### Methods and Administration

In embodiments, there is provided a method of treating mania associated with a diseased condition in a subject in need thereof, comprising administering oromucosally (for example, sublingually or buccally) an effective amount of dexmedetomidine or a pharmaceutically acceptable salt thereof to the subject. In embodiments, the treatment is effective without causing significant sedation. In embodiments, the treatment is effective without experiencing clinically significant cardiovascular effects. In embodiments, the subject having a manic episode is in an agitated state. In embodiments, the subject having a manic episode is in a non-agitated state.

In embodiments, the diseased condition is neuropsychiatric disorder such as bipolar disorder (such as bipolar I disorder and bipolar II disorder). Bipolar disorders can be diagnosed by the clinical evaluation of patients using the criteria specified in the Diagnostic and Statistical Manual (DSM-IV) of the American Psychiatric Association. This disorder is distinct from the more common form of depression, called Major Depressive Disorder, in which patients only experience recurrent episodes of depression but no mania. Episodes of mania occur in patients who suffer from bipolar disorder which is an illness characterized by alternating cycles of depression and mania.

In embodiments, there is provided a method of treating bipolar mania in a subject in need thereof, comprising administering oromucosally (for example, sublingually or buccally) an effective amount of dexmedetomidine or a pharmaceutically acceptable salt thereof to the subject. In embodiments, the treatment is effective without causing significant sedation. In embodiments, the treatment is effective without experiencing clinically significant cardiovascular effects.

In embodiments, the present disclosure provides an oromucosal composition for treating mania in a subject in need thereof, comprising an effective amount of dexmedetomidine or a pharmaceutically acceptable salt thereof, together with one or more pharmaceutically acceptable excipients and/or carriers wherein the mania is associated with a neuropsychiatric disease. In embodiments, the present disclosure provides an oromucosal composition for treating mania in a subject in need thereof, wherein said mania is associated with bipolar disorder (bipolar I disorder and bipolar II disorder).

In embodiments, the present disclosure provides an oromucosal composition for treating mania in a subject in need thereof, comprising an effective amount of dexmedetomidine or a pharmaceutically acceptable salt thereof, together with one or more pharmaceutically acceptable excipients and/or carriers wherein the mania is associated with bipolar disorder.

In embodiments, the mania is acute. In embodiments, the mania is recurring. In embodiments, the mania is a single episode. In embodiments, the acute mania is associated with acute manic and/or mixed episodes. In embodiments, mania includes hypomania. In embodiments, the mania is mixed mania. In embodiments, the mania is dysphoric mania. In embodiments, the mania is mild. In embodiments, mania is severe. Signs of mania include anxiety with depression, restlessness, affective lability, prominent irritability and emotional reactivity.

In embodiments, there is provided a method of treating mania associated with neuropsychiatric disorders in a subject in need thereof, comprising administering oromucosally (sublingually or buccally) an effective amount of dexmedetomidine or a pharmaceutically acceptable salt thereof to the subject on a daily basis for at least one month wherein said subject is in a non-agitated state. In embodiments, dexmedetomidine or a pharmaceutically acceptable salt thereof is administered for at least 2 months, at least 3 months, at least 4 months, at least 5 months, at least 6 months, at least 7 months, at least 8 months, at least 9 months, at least 10 months, at least 11 months, at least 12 months or at least one year. In embodiments, mania is associated with depression. In embodiments, the mania is associated with bipolar disorder.

In embodiments, there is provided a method of treating mania associated with neuropsychiatric disorders in a subject in need thereof, comprising administering oromucosally (sublingually or buccally) an effective amount of dexmedetomidine or a pharmaceutically acceptable salt thereof to the subject on a daily basis for at least one month wherein said subject is in an agitated state. In embodiments, dexmedetomidine or a pharmaceutically acceptable salt thereof is administered for at least 2 months, at least 3 months, at least 4 months, at least 5 months, at least 6 months, at least 7 months, at least 8 months, at least 9 months, at least 10 months, at least 11 months, at least 12 months or at least one year. In embodiments, mania is associated with depression. In embodiments, the mania is associated with bipolar disorder.

In embodiments, there is provided a method of treating mania associated with neurodegenerative disorders (such as Lewy Body or Parkinson's, dementia etc) in a subject in need thereof, comprising administering oromucosally (sublingually or buccally) an effective amount of dexmedetomidine or a pharmaceutically acceptable salt thereof to the subject on a daily basis for at least one month. In embodiments, dexmedetomidine or a pharmaceutically acceptable salt is administered for at least 2 months, at least 3 months, at least 4 months, at least 5 months, at least 6 months, at least 7 months, at least 8 months, at least 9 months, at least 10 months, at least 11 months, at least 12 months or at least one year.

In embodiments, dexmedetomidine or a pharmaceutically acceptable salt thereof is administered at a dose of about 2 μg to about 405 μg, such as about 120 μg to about 270 μg, or at a dose of about 180 μg to about 405 μg, such as about 180 μg to about 270 μg, including administering doses of about 120 μg or about 180 μg, to treat mania in a human subject. In a embodiments, the present disclosure provides methods of treating mania in a human subject with diseased condition, without also inducing significant sedation, comprising administering one or more doses of dexmedetomidine or a pharmaceutically acceptable salt thereof in a day wherein the dose of dexmedetomidine or a pharmaceutically acceptable salt is about 30 μg to about 180 μg.

In embodiments, dexmedetomidine or a pharmaceutically acceptable salt thereof is administered oromucosally at a dose of about 2 μg to about 300 μg, such as about 10 μg to about 250 μg, or at a dose of about 10 μg to about 200 μg, such as about 30 μg to about 180 μg, including administering doses of about 30 μg, 60 μg, 90 μg, 120 μg or about 180 μg, to treat mania in a human subject, without also inducing significant sedation wherein dexmedetomidine or a pharmaceutically acceptable salt thereof is administered on a daily basis for at least one month. In embodiments, dexmedetomidine or a pharmaceutically acceptable salt thereof is administered once a day. In embodiments, dexmedetomidine or a pharmaceutically acceptable salt thereof is administered at night-time on a daily basis. In embodiments, dexmedetomidine or a pharmaceutically acceptable salt thereof is administered at a dose of 120 μg at night-time on a daily basis. In embodiments, dexmedetomidine or a pharmaceutically acceptable salt thereof is administered at a dose of 180 μg at night-time on a daily basis. In embodiments, dexmedetomidine or a pharmaceutically acceptable salt thereof is also administered at day-time on an as needed basis.

In embodiments, the dosage of dexmedetomidine or a pharmaceutically acceptable salt thereof may be administered twice a day. In embodiments, the dosages of dexmedetomidine or a pharmaceutically acceptable salt thereof are administered at a dose of about 30 μg to about 90 μg during daytime (e.g., 30 μg, 45 μg, 60 μg, or 90 μg) and about 120 μg to about 180 μg during night-time (e.g., 120 μg or 180 μg). In embodiments, the dosages of dexmedetomidine or a pharmaceutically acceptable salt thereof are administered at a dose of about 30 μg to about 90 μg during daytime and 30 μg to about 90 μg during night-time. In embodiments, the dosages of dexmedetomidine or a pharmaceutically acceptable salt thereof are administered twice a day at a dose of about 120 μg to about 180 μg during daytime and about 30 μg to about 90 μg during night-time.

In embodiments, dexmedetomidine or a pharmaceutically acceptable salt thereof is administered as 60 μg per unit dose twice a day to a total dose of 120 μg. For example, a 60 μg unit dose is taken in the morning and another 60 μg unit dose is taken in the evening or night-time.

In embodiments, dexmedetomidine or a pharmaceutically acceptable salt thereof is administered oromucosally at a daily dose of about 2 μg to about 300 μg, such as about 10 μg to about 250 μg, or at a dose of about 10 μg to about 200 μg, such as about 30 μg to about 180 μg, including administering doses of about 30 μg, 60 μg, 90 μg, 120 μg or about 180 μg, to treat mania in a human subject, without also inducing significant sedation wherein dexmedetomidine or a pharmaceutically acceptable salt thereof is administered on a daily basis for at least one month. In embodiments, dexmedetomidine or a pharmaceutically acceptable salt thereof is administered once a day. In embodiments, dexmedetomidine or a pharmaceutically acceptable salt thereof is administered at night-time once a day. In embodiments, dexmedetomidine or a pharmaceutically acceptable salt thereof is administered at a dose of 120 μg at night-time once a day. In embodiments, dexmedetomidine or a pharmaceutically acceptable salt thereof is administered at a dose of 180 μg at night-time once a day. In embodiments, dexmedetomidine or a pharmaceutically acceptable salt thereof is also administered at day-time on as needed basis. In embodiments, dexmedetomidine or a pharmaceutically acceptable salt thereof is administered on an as-needed basis at a different dose than the night-time dose.

In embodiments, the present disclosure provides a method of treating mania in a human subject with the diseased condition, without also inducing significant sedation, comprising administering dexmedetomidine or a pharmaceutically acceptable salt thereof (e.g. the hydrochloride salt) as a single dose of about 30 μg, about 60 μg, about 90 μg, about 120 μg or about 180 μg, each dose administered one to five times a day. In embodiments, the treatment is effective without causing clinically significant cardiovascular effects.

In embodiments, the present disclosure provides a method of treating an acute manic episode associated with the diseased condition in a human subject, comprising oromucosally administering a film composition comprising dexmedetomidine or a pharmaceutically acceptable salt thereof (e.g. hydrochloride salt) as a single dose of 30 μg, 60 μg, 90 μg, 120 μg or 180 μg. In embodiments, an additional dose (e.g. 30 μg, 60 μg or 90 μg,) may be taken after a suitable period of time (e.g. 2-hours) in the event of persistent or recurrent mania on a daily basis for one to six times a day. In embodiments, the dexmedetomidine or a pharmaceutically acceptable salt thereof (e.g. hydrochloride salt) is administered at night-time once a day.

In embodiments, the present disclosure provides a method of treating recurring mania associated with the diseased condition in a human subject, comprising oromucosally administering a film composition comprising dexmedetomidine or a pharmaceutically acceptable salt thereof (e.g. hydrochloride salt) as a single dose of 30 μg, 60 μg, 90 μg, 120 μg or 180 μg. In embodiments, an additional dose (e.g. 30 μg, 60 μg or 90 μg) may be taken after a suitable period of time (e.g. 2-hours) in the event of persistent or recurrent mania. In embodiments, the dexmedetomidine or a pharmaceutically acceptable salt thereof (e.g. hydrochloride salt) is administered at night-time once a day. In embodiments, the dexmedetomidine or a pharmaceutically acceptable salt thereof (e.g. hydrochloride salt) is administered at day-time on as needed basis.

In embodiments, dexmedetomidine or a pharmaceutically acceptable salt thereof is administered sublingually in the form of a film. In embodiments, the film is placed under the tongue, close to the base of the tongue, on the left or right side.

In embodiments, dexmedetomidine or a pharmaceutically acceptable salt thereof is administered buccally in the form of a film, patch or tablet, particularly a film. In embodiments, the film is placed against the inner lip or cheek, close to the jaw line.

In embodiments, the present disclosure provides an oromucosal film composition for treating mania in a subject in need thereof, comprising an effective amount of dexmedetomidine or a pharmaceutically acceptable salt thereof, one or more water-soluble polymers and one or more pharmaceutically acceptable excipients and/or carriers. In embodiments, the film is mucoadhesive. In embodiments, the film has a disintegration time of about 10 seconds to about 60 seconds.

In embodiments, the present compositions are in the form of an oromucosal tablet for treating mania in a subject in need thereof, comprising an effective amount of dexmedetomidine or a pharmaceutically acceptable salt thereof, together with one or more pharmaceutically acceptable excipients and/or carriers.

In embodiments, the present compositions are in the form of an oromucosal spray formulation for treating mania in a subject in need thereof, comprising an effective amount of dexmedetomidine or a pharmaceutically acceptable salt thereof, together with one or more pharmaceutically acceptable excipients and/or carriers.

In embodiments, the present compositions are in the form of an oromucosal drop formulation for treating mania in a subject in need thereof, comprising an effective amount of dexmedetomidine or a pharmaceutically acceptable salt thereof, together with one or more pharmaceutically acceptable excipients and/or carriers.

In embodiments, the compositions are in the form of an oromucosal gel formulation for treating mania in a subject in need thereof, comprising an effective amount of dexmedetomidine or a pharmaceutically acceptable salt thereof, together with one or more pharmaceutically acceptable excipients and/or carriers.

In embodiments, dexmedetomidine or a pharmaceutically acceptable salt thereof is administered to the subject by oral route. In embodiments, the present compositions are in the form of oral tablets, orally disintegrating tablets (ODTs), effervescent tablets, capsules, pellets, pills, lozenges or troches, powders, dispersible granules, catchets, aqueous solutions, syrups, emulsions, suspensions, solutions, soft gels, dispersions and the like. In embodiments, dexmedetomidine or a pharmaceutically acceptable salt thereof is administered orally to the subject in the form of an orally disintegrating tablet.

In embodiments, dexmedetomidine or a pharmaceutically acceptable salt thereof is administered in the form of an intramuscular injection.

In embodiments, there is provided a method of treating mania in a subject in need thereof without also inducing significant sedation, comprising administering intramuscularly a therapeutically effective amount of dexmedetomidine or a pharmaceutically acceptable salt thereof to the subject on a daily basis wherein said subject is in a non-agitated state. In embodiments, mania is associated with depression. In embodiments, the mania is associated with bipolar disorder. In embodiments, mania is associated with other neuropsychiatric disorders. In embodiments, dexmedetomidine or a pharmaceutically acceptable salt is administered daily for at least 1 month, at least 2 months, at least 3 months, at least 4 months, at least 5 months, at least 6 months, at least 7 months, at least 8 months, at least 9 months, at least 10 months, at least 11 months, at least 12 months or at least one year.

In embodiments, dexmedetomidine or a pharmaceutically acceptable salt thereof is administered intramuscularly at a dose of about 10 μg to about 200 μg, e.g. about 20 μg to about 190 μg, about 30 μg to about 190 μg, about 40 μg to about 190 μg, about 50 μg to about 190 μg, about 60 μg to about 190 μg, about 70 μg to about 190 μg, about 80 μg to about 190 μg, about 90 μg to about 190 μg, about 100 μg to about 190 μg, about 110 μg to about 190 μg, about 120 μg to about 190 μg, about 130 μg to about 190 μg, about 140 μg to about 190 μg, about 150 μg to about 190 μg, about 160 μg to about 190 μg, about 170 μg to about 190 μg, about 180 μg to about 190 μg.

In embodiments, there is provided a method of treating an acute manic episode in a human subject with the diseased condition, without also inducing significant sedation, comprising administering intramuscularly an effective amount of dexmedetomidine or a pharmaceutically acceptable salt thereof to the subject. In embodiments, dexmedetomidine or a pharmaceutically acceptable salt thereof may be administered intramuscularly at a dose of about 10 μg to about 300 μg, (e.g. about 120 μg to about 190 μg). In embodiments, the diseased condition is neuropsychiatric disorder such as bipolar disorder (such as bipolar I disorder and bipolar II disorder). In embodiments, the neuropsychiatric disorder may be delirium, depression, schizophrenia; optionally the dementia or mood disorder may be in a subject with major depression or another related neuropsychiatric disorder. In embodiments, dexmedetomidine is administered to the subject on a daily basis. In embodiments, dexmedetomidine is administered to the subject on daily basis for one to six times a day. In embodiments, the treatment is effective without causing significant sedation. In embodiments, the treatment is effective without experiencing clinically significant cardiovascular effects. In embodiments, the subject is agitated. In embodiments, the subject is in a non-agitated state.

In embodiments, there is provided a method of treating a recurring manic episode in a human subject with the diseased condition, without also inducing significant sedation, comprising administering intramuscularly an effective amount of dexmedetomidine or a pharmaceutically acceptable salt thereof to the subject. In embodiments, dexmedetomidine or a pharmaceutically acceptable salt thereof may be administered intramuscularly at a dose of about 10 μg to about 200 μg, (e.g. about 120 μg to about 190 μg). In embodiments, the diseased condition is neuropsychiatric disorder such as bipolar disorder (such as bipolar I disorder and bipolar II disorder). In embodiments, the neuropsychiatric disorder may be delirium, depression, schizophrenia; optionally the dementia or mood disorder may be in a subject with major depression or another related neuropsychiatric disorder. In embodiments, dexmedetomidine is administered to the subject on a daily basis. In embodiments, dexmedetomidine is administered to the subject on daily basis for one to six times a day. In embodiments, the treatment is effective without causing significant sedation. In embodiments, the treatment is effective without experiencing clinically significant cardiovascular effects. In embodiments, the subject is agitated. In embodiments, the subject is in a non-agitated state.

In embodiments, the present disclosure provides an intramuscular injectable composition for treating mania in a subject in need thereof, comprising an effective amount of dexmedetomidine or a pharmaceutically acceptable salt thereof, together with one or more pharmaceutically acceptable excipients and/or carriers.

Young Mania Rating Scale (YMRS)—The YMRS is an 11-item, clinician-administered rating scale to assess the severity of manic symptoms before, during and after treatment. There are four items that are graded on a 0 to 8 scale (irritability, speech, thought content, and disruptive/aggressive behavior), while the remaining seven items are graded on a 0 to 4 scale. A score of 0 indicates the behavior is absent, whereas a score of 4 or 8 indicates the behavior is present and severe.

The present disclosure also provides a method of achieving a YMRS score reduction in mania for a sustained period of time in a subject with bipolar disorder or other neurological disorders (e.g. neuropsychiatric disorders, neurodegenerative disorders or so on) comprising administering to the subject a pharmaceutical composition comprising dexmedetomidine or a pharmaceutically acceptable salt thereof at a dose of about 40 μg to about 180 μg on a daily basis for at least one month. In embodiments, the mean YMRS score reduction is at least about 30%. In embodiments, the mean YMRS score reduction is about 35%. In embodiments, the mean YMRS total score reduction is about 40%. In embodiments, YMRS score reduction is about 45%. In embodiments, YMRS score reduction is about 50% from baseline. In embodiments, YMRS score reduction is more than 50%. In embodiments, the dosage may be administered for at least 2 weeks. In embodiments, the administration is followed by conventional mood stabilizer, antipsychotic or standard of care.

In embodiments, the dosage of dexmedetomidine or a pharmaceutically acceptable salt thereof may be administered twice a day. In embodiments, the dosages of dexmedetomidine or a pharmaceutically acceptable salt thereof are administered at a dose of about 30 μg to about 90 μg during daytime (e.g., 30 μg, 45 μg, 60 μg, or 90 μg) and about 120 μg to about 180 μg during night-time (e.g., 120 μg or 180 μg). In embodiments, the dosages of dexmedetomidine or a pharmaceutically acceptable salt thereof are administered at a dose of about 30 μg to about 90 μg during daytime and 30 μg to about 90 μg during night-time. In embodiments, the dosages of dexmedetomidine or a pharmaceutically acceptable salt thereof are administered twice a day at a dose of about 120 μg to about 180 μg during daytime and about 30 μg to about 90 μg during night-time.

In embodiments, dexmedetomidine or a pharmaceutically acceptable salt thereof is administered as 60 μg per unit dose twice a day to a total dose of 120 μg. For example, a 60 μg unit dose is taken in the morning and another 60 μg unit dose is taken in the evening or night-time.

In embodiments, the dose of dexmedetomidine or a pharmaceutically acceptable salt thereof is administered at night-time. In embodiments, the dose of dexmedetomidine or a pharmaceutically acceptable salt thereof is administered at day-time. In embodiments, the dose of dexmedetomidine or a pharmaceutically acceptable salt thereof is administered at night time and day-time. In embodiments, the composition comprises dexmedetomidine hydrochloride. In embodiments, dexmedetomidine or a pharmaceutically acceptable salt thereof is administered at a dose of about 120 μg on a daily basis for at least one month. In embodiments, dexmedetomidine or a pharmaceutically acceptable salt thereof is administered at a dose of about 180 μg on a daily basis for at least one month. In embodiments, the dose of dexmedetomidine or a pharmaceutically acceptable salt thereof is administered at a night-time once a day. In embodiments, the sustained period is about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In embodiments, there is provided a method of treating psychosis associated with a diseased condition in a subject in need thereof, comprising administering oromucosally (for example, sublingually or buccally) an effective amount of dexmedetomidine or a pharmaceutically acceptable salt thereof to the subject. In embodiments, the treatment is effective without causing significant sedation. In embodiments, the treatment is effective without experiencing clinically significant cardiovascular effects.

In embodiments, the psychosis is associated with a neuropsychiatric disorder selected from the group consisting of schizophrenia, schizoaffective disorder, depression, dementia and bipolar disorder optionally the dementia or mood disorder in a subject with major depressive episode or another related neuropsychiatric disorder.

In some embodiments, the psychosis is associated with diseased condition such as substance abuse disorders (e.g, alcohol, opioid and other substance withdrawal). In embodiments, the subject is in an agitated state. In embodiments, the subject is in a non-agitated state.

In embodiments, there is provided a method of treating psychosis associated with neuropsychiatric disorders in a subject in need thereof, comprising administering oromucosally (sublingually or buccally) an effective amount of dexmedetomidine or a pharmaceutically acceptable salt thereof to the subject on a daily basis for at least one month wherein said subject is in a non-agitated state. In embodiments, dexmedetomidine or a pharmaceutically acceptable salt thereof is administered for at least 2 months, at least 3 months, at least 4 months, at least 5 months, at least 6 months, at least 7 months, at least 8 months, at least 9 months, at least 10 months, at least 11 months, at least 12 months or at least one year. In embodiments, psychosis is associated with schizophrenia. In embodiments, psychosis is associated with bipolar disorder. In embodiments, psychosis is associated with schizoaffective disorder. In embodiments, psychosis is associated with depression. In embodiments, psychosis is associated with dementia. In embodiments, psychosis is associated with Parkinson's disease.

In embodiments, there is provided a method of treating psychosis associated with neurodegenerative disorders in a subject in need thereof, comprising administering oromucosally (sublingually or buccally) an effective amount of dexmedetomidine or a pharmaceutically acceptable salt thereof to the subject on a daily basis for at least one month. In embodiments, dexmedetomidine or a pharmaceutically acceptable salt is administered for at least 2 months, at least 3 months, at least 4 months, at least 5 months, at least 6 months, at least 7 months, at least 8 months, at least 9 months, at least 10 months, at least 11 months, at least 12 months or at least one year.

In embodiments, the psychosis is acute. In embodiments, the psychosis is chronic. In embodiments, the psychosis is a single episode. In embodiments, the psychosis is recurring or includes recurrent episodes. In embodiments, the acute psychosis is associated with acute psychotic episodes and/or mixed episodes.

In embodiments, dexmedetomidine or a pharmaceutically acceptable salt thereof is administered at a dose of about 2 μg to about 405 μg, such as about 120 μg to about 270 μg, or at a dose of about 180 μg to about 405 μg, such as about 180 μg to about 270 μg, including administering doses of about 120 μg or about 180 μg, to treat psychosis in a human subject.

In embodiments, dexmedetomidine or a pharmaceutically acceptable salt thereof is administered oromucosally at a dose of about 2 μg to about 300 μg, such as about 10 μg to about 250 μg, or at a dose of about 10 μg to about 200 μg, such as about 30 μg to about 180 μg, including administering doses of about 30 μg, 60 μg, 90 μg, 120 μg or about 180 μg, to treat psychosis in a human subject, without also inducing significant sedation.

In embodiments, dexmedetomidine or a pharmaceutically acceptable salt thereof is administered oromucosally at a dose of about 2 μg to about 300 μg, such as about 10 μg to about 250 μg, or at a dose of about 10 μg to about 200 μg, such as about 30 μg to about 180 μg, including administering doses of about 30 μg, 60 μg, 90 μg, 120 μg or about 180 μg, to treat psychosis in a human subject, without also inducing significant sedation wherein dexmedetomidine or a pharmaceutically acceptable salt thereof is administered on daily basis for at least one month. In embodiments, dexmedetomidine or a pharmaceutically acceptable salt thereof is administered once a day. In embodiments, dexmedetomidine or a pharmaceutically acceptable salt thereof is administered at night-time once a day. In embodiments, dexmedetomidine or a pharmaceutically acceptable salt thereof is administered at a dose of 120 μg at night-time on daily basis (e.g. once a day). In embodiments, dexmedetomidine or a pharmaceutically acceptable salt thereof is administered at a dose of 180 μg at night-time once a day. In embodiments, dexmedetomidine or a pharmaceutically acceptable salt thereof is also administered at day-time on an as needed basis. In embodiments, dexmedetomidine or a pharmaceutically acceptable administered on an as-needed basis is at a different dose than the night-time dose.

In embodiments, the present disclosure provides methods of treating psychosis in a human subject with diseased condition, without also inducing significant sedation, comprising administering oromucosally (e.g. sublingually or buccally) from about 30 μg to about 300 μg of dexmedetomidine or a pharmaceutically acceptable salt thereof on a daily basis for at least one month. In embodiments, the dose of dexmedetomidine or a pharmaceutically acceptable salt thereof is administered at a night-time.

In embodiments, the present disclosure provides a method of treating psychosis in a human subject with the diseased condition, without also inducing significant sedation, comprising administering oromucosally (e.g. sublingually or buccally) dexmedetomidine or a pharmaceutically acceptable salt thereof (e.g. the hydrochloride salt) as a single dose of about 30 μg, about 60 μg, about 90 μg, about 120 μg, about 180 μg or about 240 μg. In embodiments, the treatment is effective without causing clinically significant cardiovascular effects.

In embodiments, the present disclosure provides a method of treating psychosis associated with the diseased condition in a human subject, comprising administering oromucosally a film composition comprising dexmedetomidine or a pharmaceutically acceptable salt thereof (e.g. hydrochloride salt) as a single dose of 120 μg or 180 μg. In embodiments, an additional dose (e.g. 90 μg or 60 μg) may be taken after a suitable period of time (e.g. 2-hours) in the event of persistent or recurrent psychosis (e.g. by cutting a 180 μg or 120 μg film in half) on daily basis for one to six times a day. In a particular aspect, the treatment is effective without causing significant sedation.

In embodiments, the present disclosure provides a method of treating psychosis in a human subject with the diseased condition, without also inducing significant sedation, comprising administering oromucosally (e.g. sublingually or buccally) dexmedetomidine or a pharmaceutically acceptable salt thereof (e.g. the hydrochloride salt) as a single dose of about 30 μg, about 60 μg, about 90 μg, about 120 μg or about 180 μg on daily basis at night-time. In embodiments, the treatment is effective without causing clinically significant cardiovascular effects. In embodiments, wherein said subject is in a non-agitated state.

In embodiments, the present disclosure provides a method of treating acute psychotic episode associated with the diseased condition in a human subject, comprising oromucosally administering a film composition comprising dexmedetomidine or a pharmaceutically acceptable salt thereof (e.g. hydrochloride salt) as a single dose of 30 μg, 60 μg, 90 μg, 120 μg or 180 μg. In embodiments, an additional dose (e.g. 30 μg, 60 μg or 90 μg,) may be taken after a suitable period of time (e.g. 2-hours) in the event of persistent or recurrent psychosis on a daily basis for one to six times a day.

In embodiments, the present disclosure provides a method of treating chronic psychosis associated with the diseased condition in a human subject, comprising oromucosally administering a film composition comprising dexmedetomidine or a pharmaceutically acceptable salt thereof (e.g. hydrochloride salt) as a single dose of 30 μg, 60 μg, 90 μg, 120 μg, 180 μg or 240 μg. In embodiments, an additional dose (e.g. 30 μg, 60 μg or 90 μg) may be taken after a suitable period of time (e.g. 2-hours) in the event of persistent or recurrent psychosis on a daily basis for one to six times a day. In embodiments, wherein said subject is in a non-agitated state.

In embodiments, the disclosure provides methods of treating psychosis in a schizophrenia patient in need thereof, comprising administering oromucosally (sublingually or buccally) an effective amount of dexmedetomidine or a pharmaceutically acceptable salt thereof to the subject wherein said subject is in a non-agitated state. In embodiments, the present disclosure provides an oromucosal film composition for treating psychosis in a subject in need thereof, comprising an effective amount of dexmedetomidine or a pharmaceutically acceptable salt thereof, one or more water-soluble polymers and one or more pharmaceutically acceptable excipients and/or carriers. In embodiments, the film is mucoadhesive. In embodiments, the film has a disintegration time of about 10 seconds to about 60 seconds.

In embodiments, the present compositions are in the form of an oromucosal tablet for treating psychosis in a subject in need thereof, comprising an effective amount of dexmedetomidine or a pharmaceutically acceptable salt thereof, together with one or more pharmaceutically acceptable excipients and/or carriers.

In embodiments, the present compositions are in the form of an oromucosal spray formulation for treating psychosis in a subject in need thereof, comprising an effective amount of dexmedetomidine or a pharmaceutically acceptable salt thereof, together with one or more pharmaceutically acceptable excipients and/or carriers.

In embodiments, the present compositions are in the form of an oromucosal drop formulation for treating psychosis in a subject in need thereof, comprising an effective amount of dexmedetomidine or a pharmaceutically acceptable salt thereof, together with one or more pharmaceutically acceptable excipients and/or carriers.

In embodiments, the compositions are in the form of an oromucosal gel formulation for treating psychosis in a subject in need thereof, comprising an effective amount of dexmedetomidine or a pharmaceutically acceptable salt thereof, together with one or more pharmaceutically acceptable excipients and/or carriers.

In embodiments, the disclosure provides methods of treating psychosis in a schizophrenia patient in need thereof, comprising administering intramuscularly an effective amount of dexmedetomidine or a pharmaceutically acceptable salt thereof to the subject wherein said subject is in a non-agitated state.

In embodiments, there is provided a method of treating psychosis associated with a diseased condition in a subject in need thereof, comprising administering intramuscularly an effective amount of dexmedetomidine or a pharmaceutically acceptable salt thereof to the subject. In embodiments, the treatment is effective without causing significant sedation. In embodiments, the treatment is effective without experiencing clinically significant cardiovascular effects.

In embodiments, the psychosis is associated a neuropsychiatric disorder selected from the group consisting of schizophrenia, schizoaffective disorder, depression, dementia and bipolar disorders, optionally the dementia or mood disorder in a subject with a major depressive episode, in major mood disorder or another related neuropsychiatric disorder.

In embodiments, the psychosis is associated with diseased conditions such as substance abuse disorders (e.g, alcohol, opioid and other substance withdrawal). In embodiments, the psychosis may not be associated with substance abuse disorders. In embodiments, the subject is in an agitated state. In embodiments, the subject is in a non-agitated state.

In embodiments, there is provided a method of treating psychosis associated with neuropsychiatric disorders in a subject in need thereof, comprising administering intramuscularly an effective amount of dexmedetomidine or a pharmaceutically acceptable salt thereof to the subject on a daily basis for at least one month wherein said subject is in a non-agitated state. In embodiments, dexmedetomidine or a pharmaceutically acceptable salt thereof is administered for at least 2 months, at least 3 months, at least 4 months, at least 5 months, at least 6 months, at least 7 months, at least 8 months, at least 9 months, at least 10 months, at least 11 months, at least 12 months or at least one year. In embodiments, psychosis is associated with schizophrenia. In embodiments, psychosis is associated with bipolar disorder. In embodiments, psychosis is associated with schizoaffective disorder. In embodiments, psychosis is associated with depression. In embodiments, psychosis is associated with dementia.

In embodiments, there is provided a method of treating psychosis associated with neurodegenerative disorders in a subject in need thereof, comprising administering intramuscularly an effective amount of dexmedetomidine or a pharmaceutically acceptable salt thereof to the subject on a daily basis for at least one month. In embodiments dexmedetomidine or a pharmaceutically acceptable salt is administered for at least 2 months, at least 3 months, at least 4 months, at least 5 months, at least 6 months, at least 7 months, at least 8 months, at least 9 months, at least 10 months, at least 11 months, at least 12 months or at least one year.

In embodiments, the psychosis is acute. In embodiments, the psychosis is chronic. In embodiments, the psychosis is a single episode. In embodiments, the psychosis is recurring or includes recurrent episodes. In embodiments, the acute psychosis is associated with acute and/or mixed episodes.

In embodiments, dexmedetomidine or a pharmaceutically acceptable salt thereof is administered intramuscularly at a dose of about 10 μg to about 200 μg to treat psychosis in a human subject, without also inducing significant sedation. In embodiments, dexmedetomidine or a pharmaceutically acceptable salt thereof is administered on daily basis for at least one month. In embodiments, dexmedetomidine or a pharmaceutically acceptable salt thereof is administered once a day. In embodiments, dexmedetomidine or a pharmaceutically acceptable salt thereof is also administered at day-time on as needed basis. In embodiments, dexmedetomidine or a pharmaceutically acceptable is administered on as-needed basis at a different dose than the night-time dose.

In embodiments, the present disclosure provides methods of treating psychosis in a human subject with diseased condition, without also inducing significant sedation, comprising administering intramuscularly from about 10 μg to about 200 μg of dexmedetomidine or a pharmaceutically acceptable salt thereof on a daily basis for at least one month.

In embodiments, the present disclosure provides a method of treating psychosis in a human subject with the diseased condition, without also inducing significant sedation, comprising administering intramuscularly dexmedetomidine or a pharmaceutically acceptable salt thereof (e.g. the hydrochloride salt) as a single dose of about 10 μg to about 200 μg. In embodiments, the treatment is effective without causing clinically significant cardiovascular effects.

In embodiments, the present disclosure provides a method of treating acute psychotic episode associated with the diseased condition in a human subject, comprising administering an intramuscular composition comprising dexmedetomidine or a pharmaceutically acceptable salt thereof (e.g. hydrochloride salt) as a single dose of 10 μg, about 20 μg, about 30 μg, about 40 μg, about 60 μg, about 90 μg, about 120 μg, about 140 μg, about 160 μg, about 180 μg, about 200 μg or about 240 μg.

In embodiments, the present disclosure provides a method of treating chronic psychosis associated with the diseased condition in a human subject, comprising intramuscularly administering a composition comprising dexmedetomidine or a pharmaceutically acceptable salt thereof (e.g. hydrochloride salt) as a single dose of 10 μg, about 20 μg, about 30 μg, about 40 μg, about 60 μg, about 90 μg, about 120 μg, about 140 μg, about 160 μg, about 180 μg or about 200 μg. In some embodiments, said subject is in a non-agitated state.

In embodiments, the present compositions are in the form of an intramuscular composition for treating psychosis in a subject in need thereof, comprising an effective amount of dexmedetomidine or a pharmaceutically acceptable salt thereof, together with one or more pharmaceutically acceptable excipients and/or carriers.

In embodiments, the present compositions are in the form of an oral composition for treating psychosis in a subject in need thereof, comprising an effective amount of dexmedetomidine or a pharmaceutically acceptable salt thereof, together with one or more pharmaceutically acceptable excipients and/or carriers. In embodiments, the oral compositions are in the form of oral tablets, orally disintegrating tablets (ODTs), effervescent tablets, capsules, pellets, pills, lozenges or troches, powders, dispersible granules, sachets, aqueous solutions, syrups, emulsions, suspensions, solutions, soft gels, dispersions and the like

The Positive and Negative Syndrome Scale (PANSS) standard has been widely used in clinical trials of schizophrenia and other disorders and is considered the “gold standard” for assessment of antipsychotic treatment efficacy. To assess a patient using PANSS, an approximately 45-minute clinical interview is conducted. The patient is rated from 1 to 7 on 30 different symptoms based on the interview as well as reports of family members or primary care hospital workers. Scores are often given separately for the positive items, negative items, and general psychopathology

The present disclosure provides methods of achieving a PANSS score reduction in psychosis for a sustained period of time in a subject with schizophrenia or other neurological disorders (e.g. neuropsychiatric disorders, neurodegenerative disorders or so on) comprising administering to the subject a pharmaceutical composition comprising dexmedetomidine or a pharmaceutically acceptable salt thereof at a dose of about 120 μg to about 180 μg on a daily basis for at least one month wherein said subject is in a non-agitated state. In embodiments, the PANSS score reduction is at least about 20% to about 50% from baseline score prior to treatment with dexmedetomidine. In embodiments, the PANSS score reduction is about 25% from baseline score. In embodiments, the PANSS total score reduction is about 30% from baseline score. In embodiments, the PANSS total score reduction is about 35% points from baseline score. In embodiments, the PANSS total score reduction is about 40% points from baseline score. In embodiments, the PANSS total score reduction is about 45% points from baseline score. In embodiments, the PANSS total score reduction is about 50% points from baseline score. In embodiments, the dose of dexmedetomidine or a pharmaceutically acceptable salt thereof is administered at a night-time. In embodiments, the composition comprises dexmedetomidine hydrochloride. In embodiments, dexmedetomidine or a pharmaceutically acceptable salt thereof is administered at a dose of about 120 μg on a daily basis for at least one month. In embodiments, the dose of dexmedetomidine or a pharmaceutically acceptable salt thereof is administered at a night-time. In embodiments, dexmedetomidine or a pharmaceutically acceptable salt thereof is administered at a dose of about 180 μg on a daily basis for at least one month. In embodiments, the dose of dexmedetomidine or a pharmaceutically acceptable salt thereof is administered at a night-time. In embodiments, the sustained period is about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. The dose may be administered one or more times a day. The doses can be administered daily for longer period of time for at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 30 days, at least about 2 months, at least about 3 months, at least 4 months, at least 5 months, at least 6 months or so on.

In embodiments, there is provided a method of treating anxiety in a subject in need thereof, comprising administering oromucosally (sublingually or buccally) an effective amount of dexmedetomidine or a pharmaceutically acceptable salt thereof to the subject wherein said subject is in a non-agitated state. In some embodiments, the subject does not experience agitation. A person of ordinary skill in the art will realize that agitation and anxiety can present differently. For example, a person that is agitated is quick to frustration or anger, often feeling bothered. Agitation is characterized by feeling of restlessness that manifest in an outward, physical manner via certain behaviors, such as pacing, verbalizations, and fidgeting. Typically, these physical manifestations are not directed against anything in particular. In contrast, a person with anxiety tends to experience a fear response first, with a variety of symptoms, such as nervousness, rapid heartbeat, and sweating. Thus, anxiety may be defined as subjective experience of nervousness, worry, apprehension or restlessness, ranging from excessive concern about the present or future to feelings of panic. Compared to anxiety, agitation often manifests with a more physical component that can be seen by an observer.

In embodiments, the disclosure provides methods of treating anxiety in a schizophrenia patient in need thereof, comprising administering oromucosally (sublingually or buccally) an effective amount of dexmedetomidine or a pharmaceutically acceptable salt thereof to the subject. In embodiments, the disclosure provides methods of treating anxiety in a schizophrenia patient in need thereof, comprising administering intramuscularly an effective amount of dexmedetomidine or a pharmaceutically acceptable salt thereof to the subject.

### Combination Therapy

In embodiments, the present disclosure provides methods as disclosed herein, wherein the methods comprises one or more additional therapeutic agents. Such combination therapy may be particularly useful in the treatment of mania in various diseased conditions. The combination therapy may be useful in the treatment of psychosis in various diseased conditions.

Examples of suitable additional therapeutic agents include antidepressants such as selective serotonin reuptake inhibitors (SSRIs) that include sertraline (Zoloft), fluoxetine (Prozac, Sarafem), citalopram (Celexa); escitalopram (Lexapro), paroxetine (Paxil, Pexeva, Brisdelle), fluvoxamine (Luvox); serotonin and norepinephrine reuptake inhibitors (SNRIs) such as desvenlafaxine (Pristiq, Khedezla), duloxetine (Cymbalta), levomilnacipran (Fetzima), venlafaxine (Effexor XR); tricyclic antidepressants such as amitriptyline, amoxapine, clomipramine (Anafranil), desipramine (Norpramin), doxepin, imipramine (Tofranil), nortriptyline (Pamelor), protriptyline, trimipramine (Surmontil); tetracyclic antidepressants like maprotiline and dopamine reuptake blocker such as bupropion (Wellbutrin, Forfivo, Aplenzin); 5-HT1A or 5-HT2 or 5-HT3 receptor antagonist such as vilazodone (Viibryd); nefazodone and trazodone (Oleptro); vortioxetine (Brintellix); noradrenergic antagonist like mirtazapine (Remeron) and monoamine oxidase inhibitors such as isocarboxazid (Marplan), phenelzine (Nard), selegiline (Emsam) and tranylcypromine (Parnate).

In embodiments, the present disclosure provides a film as disclosed herein, wherein the film comprises dexmedetomidine or a pharmaceutically acceptable salt thereof together with one or more additional therapeutic agents.

The drug combinations herein may be included in a monolithic film of the present disclosure or a micro-deposition film of the present disclosure. If in a monolithic film, the present disclosure provides for the presence of all drugs in a single matrix film layer. The drugs may also be present in separate monolithic films which are then combined to provide a multi-layer film.

In embodiments, and more conveniently, the drugs are included in a micro-deposition film of this disclosure. Thus, for example, individual drug compositions may be added as discrete droplets to the surface of the film substrate (i.e. placebo film) according to the general process used and described herein to add the dexmedetomidine composition to a film substrate. The droplets may be added in any pattern to suit the desired unit dose requirements. The droplets may each include a colorant which may be the same or different for each drug composition. It may be convenient to use different colors to distinguish the different drugs on the surface of the film substrate.

In embodiments, the present disclosure provides an intramuscular injectable formulation as disclosed herein, wherein the formulation comprises dexmedetomidine or a pharmaceutically acceptable salt thereof together with one or more additional therapeutic agents.

In embodiments, both dexmedetomidine and additional active agent(s) are present as part of a single pharmaceutical composition for administration to the subject. In embodiments, the active agents are present in separate pharmaceutical compositions, e.g. for concurrent and/or sequential administration to the subject.

SmartCube® (general details of methods or systems is disclosed in for example U.S. Pat. No. 7,580,798 incorporated herein by reference in its entirety)

### Specific Embodiments

Embodiment 1. A method of treating mania in a subject in need thereof, comprising administering oromucosally a therapeutically effective amount of dexmedetomidine or a pharmaceutically acceptable salt thereof to the subject on a daily basis wherein said subject is in a non-agitated state.

Embodiment 2. A method of treating psychosis in a subject in need thereof, comprising oromucosally administering a therapeutically effective amount of dexmedetomidine or a pharmaceutically acceptable salt thereof to the subject on a daily basis wherein said subject is in a non-agitated state.

Embodiment 3. A method of treating mania in a subject in need thereof, comprising administering intramuscularly a therapeutically effective amount of dexmedetomidine or a pharmaceutically acceptable salt thereof to the subject on a daily basis wherein said subject is in a non-agitated state.

Embodiment 4. A method of treating psychosis in a subject in need thereof, comprising administering intramuscularly a therapeutically effective amount of dexmedetomidine or a pharmaceutically acceptable salt thereof to the subject on a daily basis wherein said subject is in a non-agitated state.

Embodiment 5. The method of embodiments 1 to 4, wherein dexmedetomidine or a pharmaceutically acceptable salt thereof is dexmedetomidine hydrochloride.

Embodiment 6. The method according to embodiments 1 to 5, wherein the therapeutically effective amount of dexmedetomidine hydrochloride is about 2 μg to about 300 μg.

Embodiment 7. The method according to embodiments 1 to 5, wherein the therapeutically effective amount of dexmedetomidine hydrochloride is about 10 μg to about 200 μg.

Embodiment 8. The method according to embodiments 1 to 5, wherein the therapeutically effective amount of dexmedetomidine hydrochloride is about 30 μg to about 180 μg.

Embodiment 9. The method according to embodiment 1 or embodiment 3, wherein the mania is associated with neuropsychiatric disorder selected from the group comprising bipolar illness such as bipolar disorder (e.g. bipolar I disorder and bipolar II disorder), optionally the dementia or mood disorder in a subject with major depressive episode or another related neuropsychiatric disorder.

Embodiment 10. The method according to embodiment 2 or embodiment 4, wherein the psychosis is associated with neuropsychiatric disorder selected from the group comprising schizophrenia, schizoaffective disorder, depression, dementia and bipolar disorder (e.g. bipolar I disorder and bipolar II disorder), optionally the dementia or mood disorder in a subject with major depressive episode or another related neuropsychiatric disorder.

Embodiment 11. The method according to embodiment 2 or embodiment 4, wherein the psychosis is associated with substance abuse withdrawal (e.g. alcohol, opioid or other substance abuse withdrawal).

Embodiment 12. The method according to any one of embodiments 1, 3, 5 to 9, wherein the subjects suffers from episodes of acute mania, recurring mania, or both.

Embodiment 13. The method according to any one of embodiments 1, 3, 5 to 9, wherein the subjects suffer from single episode of mania.

Embodiment 14. The method according to any one of embodiments 1, 3, 5 to 9, wherein the subjects suffer from recurrent episodes of mania.

Embodiment 15. The method according to any one of embodiments 1, 3, 5 to 9, wherein the mania is mild or severe.

Embodiment 16. The method according to any of embodiments 1, 3, 5 to 9, wherein the reduction in mania is measured using YMRS scale.

Embodiment 17. A method of achieving YMRS score reduction in mania for a sustained period of time in a subject with bipolar disorder or other neuropsychiatric disorders, comprising administering to the subject a pharmaceutical composition comprising dexmedetomidine or a pharmaceutically acceptable salt thereof at a dose of about 120 μg to about 180 μg on a daily basis for at least one month, wherein the YMRS score reduction is at least about 30% to about 50%.

Embodiment 18. The method according to any one of embodiments 2, 4, 10 and 11, wherein subject suffers from episodes of acute psychosis, chronic psychosis, or both.

Embodiment 19. The method according to any one of embodiments 2, 4, 10 and 11, wherein subject suffers from single episode or mixed episodes of psychosis.

Embodiment 20. The method according to any one of embodiments 2, 4, 10 and 11, wherein subject suffers from recurrent episodes of psychosis.

Embodiment 21. The method according to any one of embodiments 2, 4, 10 and 11, wherein severity of psychosis in the subject is assessed using PANSS scale.

Embodiment 22. A method of achieving a PANSS score reduction in psychosis for a sustained period of time in a subject with schizophrenia or other neurological disorders (e.g. neuropsychiatric disorders, neurodegenerative disorders or so on) comprising administering to the subject a pharmaceutical composition comprising dexmedetomidine or a pharmaceutically acceptable salt thereof at a dose of about 120 μg to about 180 μg on a daily basis for at least one month wherein said subject is in a non-agitated state and the PANSS score reduction is at least about 20% to about 50% from baseline score.

Embodiment 23. The method according to embodiment 17 and 22, wherein the sustained period is about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

Embodiment 24. The method according to any one of embodiments 1 to 9 and 12, wherein the subject suffers from anxiety with depression, hypomania, dysphoric mania, mixed mania, depressive episodes or combination thereof.

Embodiment 25. The method according to embodiment 1 to 4, wherein the subject is a human.

Embodiment 26. The method according to embodiment 1 to 4, wherein the dexmedetomidine or a pharmaceutically acceptable salt thereof is administered oromucosally (e.g., sublingually or buccally).

Embodiment 27. The method according to embodiment 26, wherein dexmedetomidine or a pharmaceutically acceptable salt thereof is administered sublingually in the form of a tablet, film, spray, gel or drops.

Embodiment 28. The method according to embodiment 27, wherein dexmedetomidine or a pharmaceutically acceptable salt thereof is administered in the form of a film.

Embodiment 29. The method according to embodiment 27, wherein dexmedetomidine or a pharmaceutically acceptable salt thereof is administered in the form of a spray.

Embodiment 30. The method according to embodiment 27, wherein dexmedetomidine or a pharmaceutically acceptable salt thereof is administered in the form of a tablet.

Embodiment 31. The method according to embodiment 27, wherein dexmedetomidine or a pharmaceutically acceptable salt thereof is administered in the form of a gel.

Embodiment 32. The method according to embodiment 27, wherein dexmedetomidine or a pharmaceutically acceptable salt thereof is administered in the form of a drop.

Embodiment 33. The method according to embodiment 26, wherein the dexmedetomidine or a pharmaceutically acceptable salt thereof is administered buccally in the form of a tablet, film, spray, gel or drops.

Embodiment 34. The method according to embodiment 33, wherein the dexmedetomidine or a pharmaceutically acceptable salt thereof is administered in the form of a film.

Embodiment 35. The method according to embodiments 1 to 4, wherein the subject is treated without causing significant sedation.

Embodiment 36. The method according to embodiments 1 to 4, wherein the subject is treated without experiencing clinically significant cardiovascular effects.

Embodiment 37. The method according to embodiments 1 to 4, wherein dexmedetomidine or a pharmaceutically acceptable salt thereof is administered one to six times a day.

Embodiment 38. The method according to embodiment 37, wherein dexmedetomidine or a pharmaceutically acceptable salt thereof is administered once daily.

Embodiment 39. The method according to embodiment 1 to 4, wherein dexmedetomidine or a pharmaceutically acceptable salt thereof is administered as a single dose.

Embodiment 40. The method according to embodiments 1 to 4, wherein dexmedetomidine or a pharmaceutically acceptable salt thereof is administered for at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 2 months, at least 3 months, at least 4 months, at least 5 months, at least 6 months or at least 1 year.

Embodiment 41. The method according to the preceding embodiments, wherein dexmedetomidine or a pharmaceutically acceptable salt thereof is administered at night-time once a day.

Embodiment 42. The method according to embodiment 41, further comprising administering dexmedetomidine or a pharmaceutically acceptable salt thereof in the day-time on an as-needed basis.

Embodiment 43. The method of embodiment 42, wherein the dexmedetomidine or a pharmaceutically acceptable administered on an as-needed basis is at a different dose than the night-time dose.

Embodiment 44. The method according to embodiment 43, wherein the dexmedetomidine or a pharmaceutically acceptable salt thereof is administered at a dose of 120 μg at night-time once a day.

Embodiment 45. The method according to embodiment 43, wherein the dexmedetomidine or a pharmaceutically acceptable salt thereof is administered at a dose of 180 μg at night-time once a day.

Embodiment 46. The method according to any of preceding embodiments, wherein an additional dose of dexmedetomidine or a pharmaceutically acceptable salt thereof may be taken after a suitable period of time (e.g. 2-hours) in the event of persistent or recurrent mania on a daily basis for one to six times a day

Embodiment 47. The method according to embodiments 1 to 4, wherein the subject is agitated or non-agitated.

Embodiment 48. A pharmaceutical composition for the treatment of mania in a subject in need thereof, comprising effective amount of dexmedetomidine or a pharmaceutically acceptable salt thereof, together with one or more pharmaceutically acceptable excipients and/or carriers wherein said composition is administered on a daily basis wherein said subject is in a non-agitated state.

Embodiment 49. A pharmaceutical composition for the treatment of psychosis in a subject in need thereof, comprising effective amount of dexmedetomidine or a pharmaceutically acceptable salt thereof, together with one or more pharmaceutically acceptable excipients and/or carriers wherein said composition is administered on a daily basis wherein said subject is in a non-agitated state.

Embodiment 50. The pharmaceutical composition according to embodiment 48 or embodiment 49, wherein dexmedetomidine is present as dexmedetomidine hydrochloride.

Embodiment 51. The pharmaceutical composition according to embodiment 48 or embodiment 49, wherein the composition is formulated for oromucosal (sublingual or buccal) administration.

Embodiment 52. The pharmaceutical composition according to embodiment 51, wherein the composition is formulated for sublingual administration.

Embodiment 53. The pharmaceutical composition according to embodiment 52, wherein the composition is formulated for sublingual administration in the form of a tablet, film, spray, gel or drops.

Embodiment 54. The pharmaceutical composition according to embodiment 51, wherein the composition is formulated for buccal administration in the form of a film, patch or tablet.

Embodiment 55. The pharmaceutical composition according to embodiment 53 or embodiment 54, wherein the composition is a film.

Embodiment 56. The pharmaceutical composition according to embodiment 48, wherein the mania is associated with a neuropsychiatric disorder selected from the group comprising bipolar illness such as bipolar disorder (e.g. bipolar I disorder and bipolar II disorder), optionally the dementia or mood disorder in a subject with major depressive episode or another related neuropsychiatric disorder.

Embodiment 57. The pharmaceutical composition according to embodiment 49, wherein the psychosis is associated with a neuropsychiatric disorder selected from the group comprising schizophrenia, schizoaffective disorder, depression, dementia and bipolar disorder (e.g. bipolar I disorder and bipolar II disorder) optionally the dementia or mood disorder in a subject with major depressive episode or another related neuropsychiatric disorder.

Embodiment 58. The method according to embodiment 49, wherein the psychosis is associated with substance abuse withdrawal (e.g. alcohol, opioid or other substance abuse withdrawal).

Embodiment 59. The methods/pharmaceutical composition according to any of preceding embodiments, wherein the dexmedetomidine or a pharmaceutically acceptable salt thereof is administered by intramuscular route.

Embodiment 60. A sublingual film composition for treating mania, comprising:
i. a therapeutically effective amount of dexmedetomidine or a pharmaceutically acceptable salt thereof;
ii. one or more water-soluble polymers and
iii. one or more pharmaceutically acceptable excipients and/or carriers.
   wherein said composition is administered on a daily basis and said subject is in a non-agitated state.

Embodiment 61. A sublingual film composition for treating psychosis, comprising:
i. a therapeutically effective amount of dexmedetomidine or a pharmaceutically acceptable salt thereof;
ii. one or more water-soluble polymers and
iii. one or more pharmaceutically acceptable excipients and/or carriers.
   wherein said composition is administered on a daily basis and said subject is in a non-agitated state.

Embodiment 62. The film composition according to embodiment 60 or embodiment 61, wherein dexmedetomidine is present as dexmedetomidine hydrochloride.

Embodiment 63. The film composition according to embodiment 60 or embodiment 61, in the form of dosage unit, wherein amount of dexmedetomidine or a pharmaceutically acceptable salt thereof present per unit is about 0.5 μg to about 300 μg.

Embodiment 64. The film composition according to embodiment 63, wherein said dosage is about 2 μg to about 200 μg.

Embodiment 65. The film composition according to embodiment 60 or embodiment 61, wherein the film comprises dexmedetomidine or a pharmaceutically acceptable salt thereof together with one or more additional therapeutic agents.

Embodiment 66. The film composition according to embodiment 65, wherein said additional therapeutic agents are administered simultaneously, sequentially or separated by an appropriate period of time.

Embodiment 67. A kit comprising (a) a therapeutically effective amount of dexmedetomidine or a pharmaceutically acceptable salt thereof in one or more unit dosages; (b) a finished container containing said unit doses; and (c) a label instructions stating that said dosages can be administered to treat mania and/or psychosis wherein said subject is in a non-agitated state.

Embodiment 68. The method according to embodiments 3 to 5 wherein the therapeutically effective amount of dexmedetomidine hydrochloride is about 2 μg to about 200 μg.

Embodiment 69. The method according to embodiments 3 to 5, wherein the therapeutically effective amount of dexmedetomidine hydrochloride is about 10 μg to about 180 μg.

Embodiment 70. The method according to embodiments 3 to 5, wherein the therapeutically effective amount of dexmedetomidine hydrochloride is about 30 μg to about 100 μg.

Embodiment 71. The method or the pharmaceutical composition according to any of preceding embodiments, wherein the subject is agitated.

Embodiment 72. A method of treating anxiety in need thereof, comprising administering oromucosally (sublingually or buccally) an effective amount of dexmedetomidine or a pharmaceutically acceptable salt thereof to the subject.

Embodiment 73. A method of treating anxiety in need thereof, comprising administering intramuscularly an effective amount of dexmedetomidine or a pharmaceutically acceptable salt thereof to the subject.

Embodiment 74. The method according to embodiment 72 or embodiment 73, wherein the subject is suffering from schizophrenia.

### Example 1: Dexmedetomidine Sublingual Film Formulation

TABLE 6
Dexmedetomidine deposited on the surface
of a polymer matrix film composition

| | | | |
|---|---|---|---|
| | Concen- | Concen- | |
| | tration | tration | |
| | g/100 g | g/100 g | |
| | (10 μg | (20 μg | |
| Ingredients | film) | film) | Function |

Drug-containing composition

| | | | |
|---|---|---|---|
| Dexmedetomidine | 0.136 | 0.267 | Active agent |
| hydrochloride | | | |
| Hydroxypropyl cellulose, | 0.301 | 0.593 | Film former |
| HPC-SSL (MW = 40,000) | | | |
| Hydroxypropyl cellulose | 0.301 | 0.593 | Film former |
| (MW = 140,000) | | | |
| FD&C Blue #1 Granular | 0.002 | 0.004 | Color |
| Ethyl Alcohol as a solvent | qs | qs | Solvent |

Polymer matrix composition

| | | | |
|---|---|---|---|
| Hydroxypropyl cellulose | 4.803 | 4.768 | Film former |
| (MW = 140,000) | | | |
| Hydroxypropyl cellulose, | 4.803 | 4.768 | Film former |
| HPC-SSL | | | |
| (MW = 40,000) | | | |
| Hydroxypropyl cellulose | 28.809 | 28.601 | Film former |
| (MW = 370,000) | | | |
| Fast Emerald Green Shade | 0.129 | 0.128 | Color |
| (NO. 06507) | | | |
| Sucralose, USP-NF Grade | 0.993 | 0.985 | Sweetener |
| Peppermint Oil, NF | 2.104 | 2.089 | Flavor |
| Polyethylene oxide | 57.618 | 57.202 | Film former & |
| (Sentry Polyox WSR 205 | | | Mucoadhesive |
| LEO NF) (MW = 600,000) | | | |
| Water as a solvent | qs | qs | Solvent |

(A) Process for the Preparation of Polymer Matrix:

Polymer mixture: Polyethylene oxide and fast emerald green shade were mixed in water for at least 180 minutes at about 1400 rpm to about 2000 rpm. Sucralose, hydroxypropyl cellulose (molecular weight 140K), hydroxypropyl cellulose, HPC-SSL (molecular weight 40K) and hydroxypropyl cellulose (molecular weight 370K) were added and mixed for at least 120 minutes at about 1600 rpm to 2000 rpm. Peppermint Oil was added to water and the resultant dispersion was then added to the polymer mixture and mixed for at least 30 minutes. The resultant mixture was further mixed under vacuum (248 torr) for at least for 30 minutes at a speed of 350 rpm and at temperature of 22.9° C.

Coating station: A roll was placed on an unwind stand and the leading edge was thread through guide bars and coating bars. The silicone-coated side of the liner was placed faced up. A gap of 40 millimeters was maintained between the coating bars. The oven set point was adjusted to 70° C. and the final drying temperature was adjusted to 85° C.

Coating/drying process: The polymer mixture was poured onto the liner between the guide bars and the coating bars. The liner was pulled slowly through the coating bar at a constant speed by hand until no liquid was remained on the coating bars. The liner was cut to approximately 12-inch length hand sheets using a safety knife. Each hand sheet was placed on a drying board and was tapped on the corners to prevent curl during drying. The hand sheets were dried in the oven until the moisture content was less than 5% (approximately 30 minutes) and then removed from the drying board. The coating weights were checked against the acceptance criteria, and if met, the hand sheets were then stacked and placed in a 34 inch×40 inch foil bag that was lined with PET release liner.

(B) Process for the Preparation of Deposition Solution:

FDC blue was dissolved in ethyl alcohol for at least 180 minutes. Dexmedetomidine hydrochloride was added to the ethyl alcohol solution with continuous stirring for 10 minutes at about 400 rpm to about 800 rpm. Hydroxypropyl cellulose (40K) and hydroxypropyl cellulose (140K) were added to the mixture, and stirred for at least 30 minutes until all the materials were dissolved.

(C) Process for the Preparation of Micro-Deposited Matrix:

The deposition solution obtained in Step (B) above was filled into a pipette to the required volume (determined according to the specific drug product strength of the final product). An appropriate amount (1.5 microliters=approximately 5 μg) of the deposition solution were deposited (e.g. as droplets) onto the polymer matrix obtained in Step (A), and repeated to a total of 10 times (i.e. 10 deposits/droplets) with space between each deposit to prevent merging of the deposits/droplets and allow subsequent cutting of the film into individual drug-containing units.

The film was initially die cut in individual units with dimensions of 22 mm×8.8 mm containing a single deposit of the drug-containing composition. The die cut micro-deposited matrixes were then dried in an oven for 70° C. for 10 minutes and further die cut into 10 units with each unit containing a single deposit of the drug-containing composition.

(D) Packaging:

Each defect-free unit was sealed individually into a foil pouch, which was then heat sealed. If the heat seal was acceptable the package was considered as an acceptable unit for commercial use.

Other unit strengths (e.g. 40 μg and 60 μg films) were similarly prepared by varying the concentrations of drug, polymers and colorant within the drug-containing composition. For example, the 40 μg and 60 μg, films were prepared from drug-containing compositions containing, respectively, approximately 2× and 3×, the amounts of drug, polymers and colorant that appear in the 20 μg drug-containing composition described in table 6 above.

### Example 2

TABLE 7
Dexmedetomidine deposited on the surface
of a polymer matrix film composition

| | | | | |
|---|---|---|---|---|
| | Concen- | Concen- | Concen- | |
| | tration | tration | tration | |
| | mg/unit | mg/unit | mg/unit | |
| | (80 μg | (120 μg | (180 μg | |
| Ingredients | film) | film) | film) | Function |

Drug-containing composition

| | | | | |
|---|---|---|---|---|
| Dexmedetomidine | 0.0945 | 0.142 | 0.213 | Active agent |
| hydrochloride | | | | |
| Hydroxypropyl | 0.0812 | 0.122 | 0.183 | Film former |
| cellulose, HPC-SSL | | | | |
| (MW = 40,000) | | | | |
| Hydroxypropyl cellulose | 0.0812 | 0.122 | 0.183 | Film former |
| (MW = 140,000) | | | | |
| FD&C Blue #1 Granular | 0.0008 | 0.001 | 0.002 | Color |
| Ethyl Alcohol as a | q.s | q.s. | q.s. | Solvent |
| solvent | | | | |

Polymer matrix composition

| | | | | |
|---|---|---|---|---|
| Hydroxypropyl cellulose | 0.627 | 0.627 | 0.627 | Film former |
| (MW = 140,000) | | | | |
| Hydroxypropyl | 0.627 | 0.627 | 0.627 | Film former |
| cellulose, HPC-SSL | | | | |
| (MW = 40,000) | | | | |
| Hydroxypropyl cellulose | 3.763 | 3.763 | 3.763 | Film former |
| (MW = 370,000) | | | | |
| Fast Emerald Green | 0.017 | 0.017 | 0.017 | Color |
| Shade (NO. 06507) | | | | |
| Sucralose, USP-NF | 0.130 | 0.130 | 0.130 | Sweetener |
| Grade | | | | |
| Peppermint Oil, NF | 0.275 | 0.275 | 0.275 | Flavor |
| Polyethylene oxide | 7.526 | 7.526 | 7.526 | Film former & |
| (Sentry Polyox WSR | | | | Mucoadhesive |
| 205 LEO NF) (MW = | | | | |
| 600,000) | | | | |
| Water as a solvent | qs | qs | qs | Solvent |

The formulations (80 μg, 120 μg and 180 μg) in table 7 were prepared using the same manufacturing process as described above in Example 1.

### Example 3: Efficacy and Safety of Dexmedetomidine Hydrochloride Sublingual Film in Subjects with Bipolar Mania

### Study Design:

The study enrolled approximately 382 subjects randomized 1:1:1 to dose regimens of 180 μg, 120 μg dexmedetomidine hydrochloride, or placebo stratified by age <65 and age ≥65.

Male and female adults with acute agitation associated with bipolar I or II disorder were enrolled. Subjects were domiciled in a clinical research setting or hospitalized to remain under medical supervision while undergoing screening procedures to assessed eligibility.

Subjects were randomized to 180 μg dexmedetomidine hydrochloride sublingual film or 120 μg dexmedetomidine hydrochloride sublingual film or matching placebo. Efficacy and safety assessments were conducted periodically before and after dosing.

Vital signs, pulse oximetry and ECG with rhythm strip were measured as per schedule of assessments, prior to any PK assessments. Participants were allowed water as desired 15 minutes after completion of dosing. Safety and tolerability assessments were conducted at various timepoints. Please refer to the Table 8 for Schedule of events.

Any abnormal vital sign measurement, clinical laboratory test, physical examination finding, or ECG parameter deemed clinically significant by the investigator were repeated, including test results obtained on the final study day or upon early termination. For any test abnormality deemed clinically significant, repeat analysis performed during the follow-up period and until the value returns to baseline (or within normal limits) or the investigator deemed the abnormality to be stable and no longer of clinical concern.

Approximately 4 mL of venous blood (to obtain a minimum of 1.2 mL plasma) was taken into K2-EDTA tubes at set time intervals for the determination of plasma concentrations of study drug (or placebo). The PK plasma samples were collected within 10 min of the scheduled sampling time on Day 1. Blood samples were collected (Table 8).

Placebo was chosen as a comparator to more accurately assess efficacy as well as safety and tolerability. The randomized, double-blind parallel-group design ensures the sponsor, all subjects, and study staff involved were shielded from treatment assignment and outcomes and therefore minimized any potential bias. The randomization ratio provided an additional element that ensured blinding by decreasing the odds of guessing treatment arms.

### Diagnosis and Main Criteria for Eligibility:

Inclusion Criteria
1. Male and female patients between the ages of 18 to 75 years, inclusive.
2. Patients who had met DSM-5 criteria for bipolar I or II disorder, generally hypomanic, manic or mixed episodes.
3. Patients who were judged to be clinically agitated at Screening and Baseline with a total score of ≥14 on the 5 items (poor impulse control, tension, hostility, uncooperativeness, and excitement) comprising the PANSS Excited Component (PEC).
4. Patients who had a score of ≥4 on at least 1 of the 5 items on the PEC at Baseline.
5. Patients who read, understand and provided written informed consent.
6. Patients who were in good general health prior to study participation as determined by a detailed medical history, physical examination, 12-lead ECG with rhythm strip, blood chemistry profile, hematology, urinalysis, and in the opinion of the Principal Investigator.
7. Female participants, if of child-bearing potential and sexually active, and male participants, if sexually active with a partner of child-bearing potential, who agreed to use a medically acceptable and effective birth control method throughout the study and for one week following the end of the study. Medically acceptable methods of contraception that might be used by the participant and/or his/her partner include abstinence, birth control pills or patches, diaphragm with spermicide, intrauterine device (IUD), condom with foam or spermicide, vaginal spermicidal suppository, surgical sterilization, and progestin implant or injection. Prohibited methods include: the rhythm method, withdrawal, condoms alone, or diaphragm alone.

Exclusion Criteria
1. Patients with agitation caused by acute intoxication, including positive identification of alcohol by breathalyzer or drugs of abuse (with the exception of THC) during urine screening.
2. Use of benzodiazepines or other hypnotics or antipsychotic drugs in the 4 hours before study treatment.
3. Treatment with alpha-1 noradrenergic blockers (terazosin, doxazosin, tamsulosin, alfuzosin, or prazosin) or other prohibited medications.
4. Patients judged to be at serious risk of suicide must be excluded.
5. Female patients who had a positive pregnancy test at screening or are breastfeeding.
6. Patients who had hydrocephalus, seizure disorder, or history of significant head trauma, stroke, transient ischemic attack, subarachnoid bleeding, brain tumor, encephalopathy, meningitis, Parkinson's disease or focal neurological findings.
7. History of syncope or other syncopal attacks, current evidence of hypovolemia, orthostatic hypotension (average of 1, 3 and 5 min measurements), a screening and baseline heart rate of <55 beats per minutes or systolic blood pressure <110 mmHg or diastolic BP<70 mmHg.
8. Patients with laboratory or ECG abnormalities considered clinically significant by the investigator or qualified designee [Advanced heart block (second-degree or above atrioventricular block without pacemaker), diagnosis of Sick sinus syndrome] that would had clinical implications for the patient's participation in the study.
9. Patients with serious or unstable medical illnesses. These include current hepatic (moderate-severe hepatic impairment), renal, gastroenterologic, respiratory, cardiovascular (including ischemic heart disease, congestive heart failure), endocrinologic, or hematologic disease.
10. Patients who had received an investigational drug within 30 days prior to the current agitation episode.
11. Patients who were considered by the investigator, for any reason, to be an unsuitable candidate for receiving dexmedetomidine hydrochloride, e.g. patients with a history of allergic reactions to dexmedetomidine hydrochloride.

### Study Treatments

### Method of Assigning Subjects to Treatment Groups

Upon confirmation of eligibility, subjects were randomized to 180 μg dexmedetomidine hydrochloride film or 120 μg dexmedetomidine hydrochloride film or placebo. Randomization was 1:1:1 (180 μg or 120 μg dexmedetomidine hydrochloride or placebo and stratified by age <65, age ≥65) with 125 patients assigned to each arm by a permuted block design.

Test Product, Dose, and Mode of Administration:

Dexmedetomidine hydrochloride was in a film formulation for sublingual (SL) administration. Dosing delivered 180 μg or 120 μg of dexmedetomidine hydrochloride sublingually. The product was a small, solid-dose film formulation, approximately 193.6 mm² in area and 0.7 mm thick, that dissolved in the oromucosal space within about 1-3 minutes.

### Administration

At the time of dosing, patients were instructed on how to take dexmedetomidine hydrochloride film sublingually, and that they should retained the dexmedetomidine hydrochloride film in the sublingual cavity until dissolved. The patient self-administered under the supervision of a trained staff member. If the patient was unable to self-administer, the event was recorded, and the subject's participation was concluded.

### Study Procedures

Subjects provided written informed consent before any study-related procedures were initiated, including the cessation of concomitant therapy.

The schedule of events performed during the study was provided in Table 8.

TABLE 8
Schedule of Events

| | | | Treatment Evaluation Day 1 | | | | | | | | | | | | |
|---|---|---|---|---|---|---|---|---|---|---|---|---|---|---|---|
| | Activity | | | | | | | | | | | | | | |
| | | | | | | | | | | | | | Day 2 | | |
| | | Pre- | | | | | | | | | | | Follow-Up | Day 3 | Day 7 |
| | Screening | Dose¹ | Post Dose Time¹ | | | | | | | | | | (+1) | Discharge | (+2) |
| | Time point | | | | | | | | | | | | | | |
| | Pre- | −1 hr to | 10 | 20 | 30 | 45 | 1 | 1.5 | 2 | 4 | 6 | 8 | 24 hr | | End of |
| | treatment | time 0 | min | min | min | min | hr | hr | hr | hr | hr | hr | (−9/+12 hr) | | Study |
| Informed Consent | X | | | | | | | | | | | | | | |
| Medical History | X | | | | | | | | | | | | | | |
| Demographics | X | | | | | | | | | | | | | | |
| Weight | X | | | | | | | | | | | | X | | |
| Height | X | | | | | | | | | | | | | | |
| BMI | X | | | | | | | | | | | | | | |
| Alcohol Breathalyzer | X | | | | | | | | | | | | | | |
| MINI | X | | | | | | | | | | | | | | |
| Physical Exam | X | | | | | | | | | | | | X | | |
| Safety Labs² | X | | | | | | | | | | | | | X | X |
| ECG with rhythm | X | X | | | | | | | X | | | | X | | |
| strip ³ | | | | | | | | | | | | | | | |
| Pulse oximetry | | X | | | X | | X | | X | X | | X | | | |
| Resting vital signs⁴ | X | X | | | X | | X | | X | X | X | X | X | X | X |
| Orthostatic vital | X | X | | | | | | | X | X | | X | X | X | X |
| signs⁴ | | | | | | | | | | | | | | | |
| Admit to Unit | X | | | | | | | | | | | | | | |
| Inclusion/Exclusion | X | X | | | | | | | | | | | | | |
| criteria | | | | | | | | | | | | | | | |
| Randomization | | X | | | | | | | | | | | | | |
| Study drug | | X | | | | | | | | | | | | | |
| administration⁹ | | | | | | | | | | | | | | | |
| YMRS | | X | | | | | | | | | | | X | | |
| PCRS⁵ | X | X | | | | | | | X | | | | X | | |
| PEC⁵ | X | X | X | X | X | X | X | X | X | X | X | X | X | | |
| ACES⁵ | | X | | | | | | | X | X | | X | | | |
| CGI-Severity⁶ | X | X | | | | | | | | | | | | | |
| CGI-Improvement⁶ | | | | | X | | X | | X | X | | | | | |
| C-SSRS | X | X | | | | | | | | | | | X | X | |
| Buccal (SL) | | | | | X | | | | X | X | | | X | | |
| assessment for local | | | | | | | | | | | | | | | |
| irritation⁷ | | | | | | | | | | | | | | | |
| Likert Scales | | | | X | | | | | | | | | | | |
| Likability Questions | | | | X | | | | | | | | | | | |
| Pharmacokinetic | | | | | | | X | | | X | | X | | | |
| Sampling⁸ | | | | | | | | | | | | | | | |

| | | | | | | |
|---|---|---|---|---|---|---|
| Concomitant Meds | X | X | X | X | X | X |
| Adverse Events | X | X | X | X | X | X |

Notes to the Schedule of Events:
¹Pre-dose assessments had a window of 60 minutes prior to dose with the exception of PEC and ACES which were performed within 15 minutes of dosing (15 to 0 min). All post-dose assessments had a window of −5/+15 minutes through the 1.5 hour assessments, −5/+25 minutes for the 2 hour assessments (with the exception of the PEC which had a +/−5 minute window) and ±30 minutes for the 4, 6 and 8 hour assessments and YMRS could be performed at any time.
²Safety Labs included chemistry, hematology, urinalysis, UDS (local lab, only conducted at screening), alcohol breathalyzer (only conducted at screening), and urine pregnancy (only conducted at screening) Screening/enrollment labs: local labs drawn within 7 days prior to screening might suffice with the exception of urine drug screen. If results not available on the same day, a ‘desktop’ or non-CLIA test might be performed; to confirm, results from a CLIA-certified laboratory should be recorded once available. Central Labs should be performed on Screening, Day 3 and Day 7.
³ ECG for pre-dose does not need to be repeated if screening ECG was conducted on the day of dosing. ECGs collected following treatment were performed prior to PK assessments.
⁴Resting (recumbent) vital signs (SBP, DBP and HR) were taken upon having the subject recumbent for 5 min at Screening, Pre-dose and at 30 min, 1, 2, 4, 6, 8 and 24 hours post dose, as well as Day 3 and Day 7. Triplicate measurements were performed in case of Systolic BP <90 mmHg, Diastolic BP <60 mmHg or Pulse <60 bpm. Orthostatic measurements (SBP, DBP, HR, respiratory rate) were taken upon having the subject stand, with measurements taken after 1, 3 and 5 minutes and temperature were taken at Screening, Pre-dose, 2, 4, 8 and 24 hours post first dose, as well as Day 3 and Day 7.
⁵PEC was performed at Screening, Pre-dose (within 15 min prior to dose) and at 10, 20, 30, 45 min; 1, 1.5, 2, 4, 6, 8 and 24 hours post dose. The PCRS must be performed prior to PEC rating, when required. ACES was performed at Pre-dose (within 15 min of dose), 2, 4 and 8 hrs post dose.
⁶CGI-Severity was performed at Screening and pre-dose. CGI-Improvement was performed at 30 minutes, 1, 2 and 4 hours post dose.
⁷Buccal examined at 30 min, 2, 4 and 24 hr post-dose for local irritation.
⁸PK blood samples were collected 1, 4, and 8 hr (while awake) after dose. A sample might not be collected if the Physician indicated in source documents that the patient was in a mental state that was not conducive to PK sample collection. Non-compliance or refusal of all or any PK draw was not exclusionary nor result in ET. Vital signs were to be done prior to PK sample draws, when performed at the same timepoints.
⁹The investigator might chose to re-dose the patient after the 2 hour post-dose assessments are performed if the PEC change from baseline is ≤40%. Patients could re-dosed after completing the 2 hour post first dose assessments. Repeat dosing administers half of a film. Patients could redosed twice in the 12 hour period post first dose. All assessments listed in this Schedule of Events at the 2 hour post first dose timepoint should be repeated at 2 hours post every re-dose. Assessments at 4, 6, or 8 hour post first dose that occur within 1 hour of a post re-dose assessment were not required to be performed

### Study Assessments

### Efficacy

The effect of study drug was evaluated using several validated instruments as described below.

### PANSS—Excitatory Component (PEC)

### Young Mania Rating Scale (YMRS)

The YMRS is an 11-item scale evaluating mania symptoms based on the patient's subjective report of their clinical condition. It was used to characterize the patient population enrolled in the study.

### Safety

Safety was assessed during the study by the monitoring and recording of AEs, clinical laboratory test results (hematology, biochemistry, and urinalysis), vital sign measurements (systolic and diastolic blood pressures, heart rate measured as pulse, respiratory rate, and temperature), ECG, and physical examination findings. Should a known safety issue be identified (e.g. a high incidence of severe hypotension or bradycardia in the active 180 μg dose arm or the 120 μg arm), the DSMB notified the sponsor. Should this occur, sponsor notified FDA, and sponsor might chose to continue dosing the patients at a lower dose.

### Pharmacokinetics

Blood samples (4 ml) were collected per Table 8—Schedule of Events. For each subject, up to 3 blood samples (12 mL of blood) were collected during the study for PK analysis. In addition, approximately 30 mL of blood was collected at screening, approximately 15 mL of blood was collected at Day 3 Discharge, and approximately 15 mL of blood was collected at Day 7(+2) for clinical laboratory testing. The total volume of blood collected during the study was expected to be approximately 72 mL. For each subject, up to 3 blood samples (12 mL of blood) were collected during the study for PK analysis. In addition, approximately 30 mL of blood was collected at screening, approximately 15 mL of blood was collected at Day 3 Discharge, and approximately 15 mL of blood was collected at Day 7(+2) for clinical laboratory testing. The total volume of blood collected during the study was expected to be approximately 72 mL.

### Statistical Analyses

### Pharmacokinetic Analyses

Plasma concentrations and concentration-time data for dexmedetomidine were used to calculate PK parameters; these data and results were reported separately. Details regarding the analyses of PK data were described in a separate PK SAP. The separate SAP for the PK analyses was prepared and finalized prior to database lock.

### Safety Analyses

All safety analyses were performed using the Safety Population. All subjects who received at least one dose of study drug were included in the population for safety analysis. Adverse events (AEs) were characterized by type, severity, seriousness, and relationship to treatment. Adverse events were coded by preferred term and system organ class using MedDRA version 20.0.

### Efficacy Analyses

The primary efficacy endpoint of the study was the absolute change from baseline in the PEC total score at 120 min. The intent to treat population was analyzed and consist of all patients who took any study medication and who had both baseline and at least 1 efficacy assessment after dosing.

### Results Summary:

### Demographics

The demographics and baseline characteristics are shown below in Table 9.

TABLE 9
Demographics

| | Dexmedetomidine | | | |
|---|---|---|---|---|
| | sublingual film | | | |
| | 180 μg | 120 μg | Placebo | Overall |
| | (N = 126) | (N = 129) | (N = 126) | (N = 381) |
| Mean age (years) | 46.0 | 45.7 | 45.1 | 45.6 |
| | (11.91) | (11.32) | (11.13) | (11.43) |
| Female N | 44 | 52 | 44 | 140 |
| (%) | (34.9) | (40.3) | (34.9) | (36.7) |
| Race (% white/% | 38.9/61.1 | 44.4/55.6 | 39.7/60.3 | 41.0/59 |
| non-white) | | | | |
| BMI | 32.53 | 31.24 | 32.56 | 32.10 |
| | (7.8) | (7.6) | (7.4) | (7.6) |
| Diagnosis: Depressed | 22% | 16% | 21% | 20% |
| Diagnosis: Hypomania | 4% | 11% | 8% | 8% |
| Diagnosis: Mania | 47% | 46% | 50% | 47% |
| Diagnosis: Mixed | 24% | 21% | 17% | 21% |
| Episodes | | | | |
| Diagnosis: Unspecified | 3% | 6% | 4% | 4% |
| Baseline PEC means | 18 | 18 | 17.9 | NA |

3. Efficacy

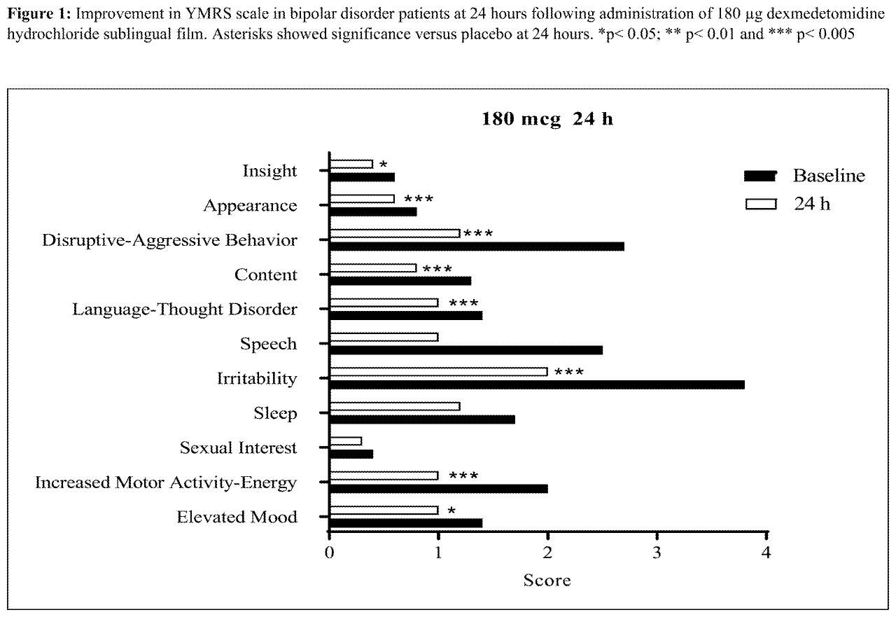

Dexmedetomidine sublingual film significantly improved the severity of bipolar mania from baseline as measured by YMRS scale. Key efficacy findings at 24 hours post-dose are presented in the FIG. 1 and tabulated below (table 10):

TABLE 10
Change from Baseline of YMRS (Intent-to-Treat Population)

| | | | |
|---|---|---|---|
| | 180 μg | 120 μg | |
| | Dexmedetomidine | Dexmedetomidine | |
| Time Point | sublingual film | sublingual film | Placebo |
| Statistics | (N = 126) | (N = 126) | (N = 126) |

Pre-Dose (Baseline

| | | | |
|---|---|---|---|
| n | 126 | 126 | 126 |
| Mean (SD) | 18.3 (7.38) | 18.0 (7.11) | 19.0 (8.11) |
| Median | 18.0 | 18.0 | 18.0 |
| Min, Max | 2, 42 | 4, 40 | 5, 50 |

24 Hours Post-Dose

| | | | |
|---|---|---|---|
| n | 124 | 125 | 125 |
| Mean (SD) | 10.9 (6.64) | 11.5 (6.54) | 14.2 (8.83) |
| Median | 10.0 | 11.0 | 12.0 |
| Min, Max | 0, 33 | 0, 27 | 0, 48 |

Change from Baseline

| | | | |
|---|---|---|---|
| n | 124 | 125 | 125 |
| Mean (SD) | −7.4 (6.96) | −6.6 (6.33) | −4.8 (6.74) |
| Median | −6.0 | −6.0 | −4.0 |
| Min, Max | −36, 5 | −22, 7 | −39, 11 |
| LS Mean, SE [1 | −7.5, 0.5 | −6.9, 0.5 | −4.4, 0.5 |
| LSM Difference, SE | −3.1, 0.7 | −2.5, 0.7 | |
| [2] | | | |
| 95% CIs [2] | −4.5, −1.7 | −3.9, −1.1 | |
| p value [3] | <0.0001 | 0.0005 | |

Young Mania Rating Scale (YMRS) is an 11item scale evaluating mania symptoms based on the patient’s subjective report of their clinical condition.
[1 Least square (LS) mean and standard error (SE) per treatment group.
[2] Treatment Effect: Least square mean (LSM) difference, standard error (SE), and 95% confidence intervals (CIs) between dexmedetomidine sublingual film and Placebo.
[3] p value comparing dexmedetomidine sublingual film and Placebo.

### CONCLUSION

Dexmedetomidine sublingual film treatment significantly improved mania from baseline as measured by YMRS in bipolar disorder patients. As given in FIG. 1, the most robust effects were measured on motor activity, irritability, thought disorder, content, aggressive behavior and appearance. The treatment effect shows a decrease of 3.1+/−0.7 SE for 180 ug and a decrease of 2.5+/−0.7 SE for 120 ug. These data show that a YMRS reduction of about 2 to 3 is achieved (approximately 30% to 40% reduction). By removing the EC (excited component) items from the PANSS score, the effect of dexmedetomidine in non-agitated patients is identified. Here those data confirm that dexmedetomidine reduces mania in non-agitated patients.

### Example 4: Treatment of Anxiety and Psychosis in Schizophrenia Patients

Schizophrenia patients were administered 120 μg and 180 μg doses in a film and monitored over 24 hours. The PANSS total minus PEC was calculated. The results are shown in table 11.

| | | | Treatment groups | | |
|---|---|---|---|---|---|
| | | | Dex | Dex | |
| | | | 180 μg | 120 ug | Placebo |
| Baseline | | N | 126 | 129 | 126 |
| | | Mean | 68.52 | 69.36 | 68.15 |
| | | Std | 12.03 | 12.32 | 10.78 |
| | | Median | 68 | 71 | 68 |
| | | Min | 43 | 40 | 45 |
| | | Max | 106 | 99 | 98 |
| 6 | Analysis | N | 125 | 127 | 124 |
| Hours | Value | Mean | 61.78 | 62.12 | 64.07 |
| | | Std | 11.83 | 11.69 | 12 |
| | | Median | 62 | 62 | 63 |
| | | Min | 36 | 38 | 41 |
| | | Max | 91 | 87 | 102 |
| | Change | N | 125 | 127 | 124 |
| | from | Mean | −6.73 | −7.35 | −4.17 |
| | Baseline | Std | 7.89 | 7.12 | 6.46 |
| | | Median | −5 | −6 | −4 |
| | | Min | −30 | −32 | −31 |
| | | Max | 12 | 7 | 16 |
| | | LSMean, | −6.9, 0.6 | −7.0, 0.6 | −4.4, 0.6 |
| | | SE | | | |
| | | LSMean | −2.5, 0.8 | −2.6, 0.8 | |
| | | Differ- | | | |
| | | ence, SE | | | |
| | | 95% CI | −4.1, −0.9 | −4.3, −1.0 | |
| | | P-value | 0.0029 | 0.0016 | |
| | | (vs. | | | |
| | | Placebo) | | | |
| 24 | Analysis | N | 125 | 127 | 125 |
| Hours | Value | Mean | 61.17 | 62.73 | 62.39 |
| | | Std | 11.45 | 11.72 | 12.33 |
| | | Median | 62 | 63 | 62 |
| | | Min | 37 | 38 | 41 |
| | | Max | 88 | 89 | 99 |
| | Change | N | 125 | 127 | 125 |
| | from | Mean | −7.46 | −6.92 | −5.58 |
| | Baseline | Std | 8.09 | 6.32 | 7.51 |
| | | Median | −6 | −6 | −5 |
| | | Min | −31 | −26 | −29 |
| | | Max | 10 | 10 | 23 |
| | | LSMean, | −7.6, 0.6 | −6.5, 0.6 | −5.8, 0.6 |
| | | SE | | | |
| | | LSMean | −1.7, 0.9 | −0.7, 0.8 | |
| | | Differ- | | | |
| | | ence, SE | | | |
| | | 95% CI | −3.5, −.1 | −2.4, 0.1 | |
| | | P-value | 0.0404 | 0.4062 | |
| | | (vs. | | | |
| | | Placebo) | | | |

Conclusion: The patients showed significant improvement PANSS total minus PEC that demonstrates Dexmedetomidine sublingual film treatment significantly improved psychosis from baseline as shown in table 11.

### Example—5: Antipsychotic Effect of Dexmedetomidine Hydrochloride in Mice Using Smart-Cube System

To confirm that dexmedetomidine has anti-psychotic effects, we used the SmartCube® system (Psychogenics, Inc., Paramus, NJ; See also U.S. Pat. No. 7,580,798 incorporated herein by reference in its entirety). This system uses features derived from mouse behavioral data to classify compounds for their ability to treat neuro-psychiatric symptoms by comparing the features to a proprietary reference database of behavioral feature sets that are linked to classes of marketed drugs known to treat neuro-psychiatric symptoms. Thus the system can be used as a model to identify the psychiatric effect of a compound by comparing the effects of the compounds against drugs with known validated effects.

By comparing the responses of animals to known drugs, the test drug can be categorized according to its function; for example, hallucinogen, anxiogenic, analgesic, cognitive enhancer, psychostimulant, mood stabilizer, high dose anti-psychotic, anti-psychotic, sedative/hypnotic, anxiolytic, high dose antidepressant, antidepressant.

Once all features are extracted from the raw data through an automated pipeline, proprietary bioinformatics algorithms are used to decorrelate groups of features and find the combination of values that best separate different groups of interest. For each compound, at each dose, they system provides a probability that the drug is active and breaks down such putative activity into the different classes of interest.

SmartCube reference data used herein include anti-psychotics tested at several dose ranges. “Anti-psychotic” versus “high dose anti-psychotic” as indicated in the legend, reflects the notion that anti-psychotics reference data is dose-dependent. Anti-psychotics, when administered at higher doses, can engage additional receptor systems and thus affect mouse behavior differently.

### Materials and Methods:

Animals: Male C57/B16 mice (N=12 per group) from Taconic Laboratories were used. Upon receipt, mice were group-housed in OPTI mouse ventilated cages with 4 mice per cage. Mice were acclimated to the colony room for at least one week prior to test and subsequently tested at approximately 8-9 weeks of age. All animals were examined, handled, and weighed prior to initiation of the study to assure adequate health and suitability and to minimize non-specific stress associated with manipulation.

During the course of the study, 12/12 light/dark cycles were maintained. The room temperature was maintained between 20 and 23° C. with a relative humidity maintained around between 30-70%. Chow and water were provided ad libitum for the duration of the study.

Animals were acclimated to the vivarium for up to one week prior to commencing study. Room temperature and humidity was recorded continuously in the holding room. The experimenter(s) were blind to the treatment distribution. The behavioral tests were conducted according to established protocols approved by the IACUC committee and PGI's Standard Operation Procedures (SOP). The standard safety precautions were applied to all studies. Personnel working in the animal room and laboratory wore protective clothing.

Treatment: 7 groups (N=12 per group)

All compounds tested were formulated in NP3 (vehicle solution): 5% Pharmasolve; 30% P3 (1:1:1 PEG200:PEG400:propylene glycol); 65% saline; pH is 5.1-6.

All SmartCube runs were conducted with NP3 as vehicle and under identical settings.
Test groups were:
Vehicle: NP3
Dexmedetomidine at 0.001, 0.002, 0.005, 0.01, 0.02, 0.05 mg/kg
   Data on the reference compounds used herein (Guanfacine and Clonidine) were provided by Psychogenics.
   Test compounds were injected intraperitoneally (IP) for 15 min before animals were placed in SmartCube for assessment.

### Explanation of SmartCube Legend:

Vehicle: The activity profile of mice injected Intraperitonealy (IP) with vehicle (NPS) Unknown: This label is assigned when SmartCube algorithm can differentiate and classify mouse behaviour from animals on drug versus vehicle control but cannot assign a drug-class signature.
Antipsychotic: SmartCube classifies the mouse behaviour with test compound as similar to treatment with marketed anti-psychotics at therapeutically relevant doses.
High Dose Antipsychotic: SmartCube classifies the mouse behaviour on test compound as similar to treatment with marketed anti-psychotics at doses that are considered as high therapeutically. High doses of anti-psychotics often cause sedation.
Antidepressant: SmartCube classifies the mouse behaviour with test compound as similar to treatment with marketed anti-depressants at therapeutically relevant doses.
High dose Antidepressant: SmartCube classifies the mouse behaviour on test compound as similar to treatment with marketed anti-depressants at doses that are considered high therapeutically.
Side Effects: SmartCube classifies the mouse behaviour with test compound as similar to some of the side effects observed with high doses of therapeutically active compounds. Side effects can be for example severe sedation, impaired locomotion or seizures.

### Results:

The results for the class analyses are presented as standardized bar charts with percentages that sum to 100 for each dose. The percentage indicates the probability the classifier can differentiate between the vehicle group and test group. The pattern indicates what Class signature was assigned.

### Dexmedetomidine has an Antipsychotic Signature in SmartCube.

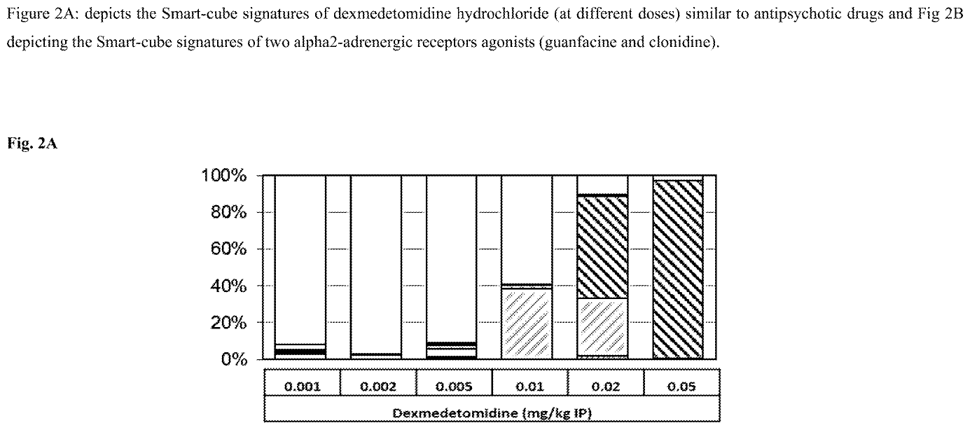

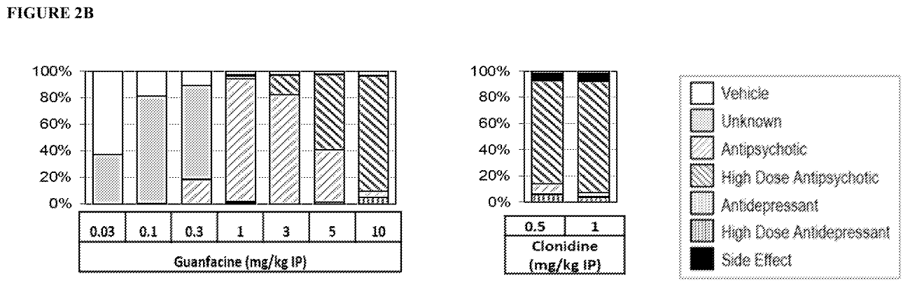

Smart-Cube signatures from mice dosed (IP mg/kg; N=12 per group) with increasing concentrations of dexmedetomidine, an alpha2-adrenergic receptor agonist (FIG. 2 (A). Smart-Cube deep learning classifiers assign activity signatures by comparing phenotypic behavior of mice injected with dexmedetomidine to a library of reference data obtained with known compounds. At 10, 20 and 50 mcg/kg (0.01, 0.02, 0.05 mg/kg), Smart-Cube dose-dependently classifies the dexmedetomidine group with increased accuracy (compared to vehicle) and assigns an antipsychotic signature. With higher doses of dexmedetomidine the antipsychotic signature changes to that of a high dose of anti-psychotic.

At 1, 2 and 5 mcg/kg SmartCube cannot distinguish the mouse behaviour from behaviour of mice on vehicle. This does not mean Dexmedetomidine at these concentrations is inactive, it merely indicates the observed behavioural changes in the mice are too subtle to detect by the classifier.

Reference data from Psychogenics showing that Guanfacine and Clonidine, both agonists of alpha2-adrenergic receptors exhibit similar signatures in a dose-dependent manner. Doses are in mg/kg (FIG. 2(B)). At therapeutically effective doses the classifier is unable to assign signature to Guanfacine whereas at higher doses the anti-psychotic signature transitions to that of a high dose anti-psychotic.

These data confirm that dexmedetomidine changes behavioral features similar to validated anti-psychotics used to treat humans.

1. A method of treating mania or hypomania in a subject in need thereof, comprising administering oromucosally a therapeutically effective amount of dexmedetomidine or a pharmaceutically acceptable salt thereof to the subject on a daily basis wherein said subject is in a non-agitated state.

2. A method of treating psychosis in a subject in need thereof, comprising administering oromucosally a therapeutically effective amount of dexmedetomidine or a pharmaceutically acceptable salt thereof to the subject on a daily basis wherein said subject is in a non-agitated state.

3. A method of treating mania or hypomania in a subject in need thereof, comprising administering intramuscularly a therapeutically effective amount of dexmedetomidine or a pharmaceutically acceptable salt thereof to the subject on a daily basis wherein said subject is in a non-agitated state.

4. A method of treating psychosis in a subject in need thereof, comprising administering intramuscularly a therapeutically effective amount of dexmedetomidine or a pharmaceutically acceptable salt thereof to the subject on a daily basis wherein said subject is in a non-agitated state.

5. The method according to any of claims 1 to 4, wherein dexmedetomidine or a pharmaceutically acceptable salt thereof is dexmedetomidine hydrochloride.

6. The method according to any of claims 1 to 5, wherein the therapeutically effective amount of dexmedetomidine hydrochloride is about 2 μg to about 300 μg.

7. The method according to any of claims 1 to 5, wherein the therapeutically effective amount of dexmedetomidine hydrochloride is about 10 μg to about 200 μg.

8. The method according to any of claims 1 to 5, wherein the therapeutically effective amount of dexmedetomidine hydrochloride is about 30 μg to about 180 μg.

9. The method according to any of claims 1 to 5, wherein the therapeutically effective amount of dexmedetomidine hydrochloride is about 30 μg to about 100 μg.

10. The method according to claims 1 and 3, wherein the mania or hypomania is associated with neuropsychiatric disorder selected from the group comprising bipolar illness such as bipolar disorder (e.g. bipolar I disorder and bipolar II disorder).

11. The method according to claims 2 and 4, wherein the psychosis is associated with a neuropsychiatric disorder selected from the group consisting of schizophrenia, bipolar illness, delirium, depression, including dementia or mood disorder in subject with major depression, preferably schizophrenia.

12. The method according to claims 2 and 4, wherein the psychosis is associated with substance abuse withdrawal and the substance is an alcohol or opioid.

13. The method according to any one of claims 1, 3, 5 to 10, wherein the subjects suffers from episodes of acute mania, recurring mania, or both.

14. The method according to any one of claims 2, 4 to 9, 11 and 12, wherein the subject suffers from episodes of acute psychosis, chronic psychosis, or both.

15. The method according to any one of claims 1, 3, 5 to 10, wherein the subject suffers from hypomania, dysphoric mania, mixed mania, mania associated with depressive episodes, or combinations thereof.

16. The method according to any of claims 1 to 4, wherein the subject is a human.

17. The method according to claim 1 or claim 2, wherein the oromucosal administration is sublingual or buccal.

18. The method according to claim 17, wherein dexmedetomidine or a pharmaceutically acceptable salt thereof is administered sublingually in the form of a tablet, film, spray, gel or drops.

19. The method according to claim 18, wherein dexmedetomidine or a pharmaceutically acceptable salt thereof is administered in the form of a film.

20. The method according to claim 18, wherein dexmedetomidine or a pharmaceutically acceptable salt thereof is administered in the form of a spray.

21. The method according to claim 18, wherein dexmedetomidine or a pharmaceutically acceptable salt thereof is administered in the form of a tablet.

22. The method according to claim 18, wherein dexmedetomidine or a pharmaceutically acceptable salt thereof is administered in the form of a gel.

23. The method according to claim 18, wherein dexmedetomidine or a pharmaceutically acceptable salt thereof is administered in the form of a drop.

24. The method according to claim 17, wherein the dexmedetomidine or a pharmaceutically acceptable salt thereof is administered buccally in the form of a tablet, film, spray, gel or drops.

25. The method according to claim 24, wherein the dexmedetomidine or a pharmaceutically acceptable salt thereof is in the form of a film.

26. The method according to any of claims 1 to 4, wherein the subject is treated without causing significant sedation.

27. The method according to any of claims 1 to 4, wherein the subject is treated without experiencing clinically significant cardiovascular effects.

28. The method according to any of claims 1 to 4, wherein dexmedetomidine or a pharmaceutically acceptable salt thereof is administered one to six times a day.

29. The method according to claim 28, wherein dexmedetomidine or a pharmaceutically acceptable salt thereof is administered once daily.

30. The method according to any of the preceding claims, wherein dexmedetomidine or a pharmaceutically acceptable salt thereof is administered for at least one week, two weeks, three weeks, one month, at least two months, at least three months, at least four months, at least five months, at least six months, at least seven months, at least eight months, at least nine months, at least ten months, at least eleven months, or at least one year.

31. The method according to any of the preceding claims, wherein dexmedetomidine or a pharmaceutically acceptable salt thereof is administered at night-time once a day.

32. The method according to claim 31, further comprising administering dexmedetomidine or a pharmaceutically acceptable salt thereof in the day-time on an as-needed basis.

33. The method according to claim 31 or 32, wherein the dexmedetomidine or a pharmaceutically acceptable administered on as-needed basis is at a different dose than the night-time dose.

34. The method according to claim 33, wherein the dexmedetomidine or a pharmaceutically acceptable salt thereof is administered at a dose of 120 μg at night-time.

35. The method according to claim 33, wherein the dexmedetomidine or a pharmaceutically acceptable salt thereof is administered at a dose of 180 μg at night-time.

36. A method of achieving YMRS score reduction in mania for a sustained period of time in a subject with bipolar disorder or other neuropsychiatric disorders, comprising administering to the subject a pharmaceutical composition comprising dexmedetomidine or a pharmaceutically acceptable salt thereof at a dose of about 120 μg to about 180 μg on a daily basis for at least one month, wherein YMRS score reduction is at least about 30% to about 50%.

37. A method of achieving a PANSS score reduction in psychosis for a sustained period of time in a subject with schizophrenia or other neurological disorders (e.g. neuropsychiatric disorders, neurodegenerative disorders or so on) comprising administering to the subject a pharmaceutical composition comprising dexmedetomidine or a pharmaceutically acceptable salt thereof at a dose of about 120 μg to about 180 μg on a daily basis for at least one month wherein said subject is in a non-agitated state and the PANSS score reduction is at least about 20% to about 50% from baseline score.

38. The method according to claims 36 and 37, wherein the sustained period is about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

39. A pharmaceutical composition for the treatment of mania in a subject in need thereof, comprising effective amount of dexmedetomidine or a pharmaceutically acceptable salt thereof, together with one or more pharmaceutically acceptable excipients and/or carriers wherein said composition is administered on a daily basis wherein said subject is in a non-agitated state.

40. A pharmaceutical composition for the treatment of psychosis in a subject in need thereof, comprising effective amount of dexmedetomidine or a pharmaceutically acceptable salt thereof, together with one or more pharmaceutically acceptable excipients and/or carriers wherein said composition is administered on a daily basis wherein said subject is in a non-agitated state.

41. The pharmaceutical composition according to claim 39 or 40, wherein dexmedetomidine is present as dexmedetomidine hydrochloride.

42. The pharmaceutical composition according to claim 39-41, wherein the composition is formulated for oromucosal (sublingual or buccal) administration

43. The pharmaceutical composition according to claim 42, wherein the composition is formulated for sublingual administration in the form of a tablet, film, spray, gel or drops.

44. The pharmaceutical composition according to claim 42, wherein the composition is formulated for buccal administration in the form of a film, patch or tablet.

45. The pharmaceutical composition according to claim 43 or 4, is in the form of a film.

46. The pharmaceutical composition according to claim 39, wherein the mania is associated with a neuropsychiatric disorder selected from the group comprising bipolar illness such as bipolar disorder.

47. The pharmaceutical composition according to claim 40, wherein the psychosis is associated with a neuropsychiatric disorder selected from the group comprising schizophrenia, schizoaffective disorder, depression, dementia and bipolar disorder (e.g. bipolar I disorder and bipolar II disorder).

48. The pharmaceutical composition according to claim 40, wherein the psychosis is associated with substance abuse withdrawal (e.g. alcohol, opioid or other substance abuse withdrawal).

49. The pharmaceutical composition according to claim 39 or 40, wherein the dexmedetomidine or a pharmaceutically acceptable salt thereof is administered by intramuscular route.

50. The pharmaceutical composition according to claim 49, wherein the amount of dexmedetomidine or a pharmaceutically acceptable salt thereof is about 2 μg to about 100 μg.

51. A sublingual film composition for treating mania in a subject in need thereof, comprising:
i. a therapeutically effective amount of dexmedetomidine or a pharmaceutically acceptable salt thereof,
ii. one or more water-soluble polymers and
iii. one or more pharmaceutically acceptable excipients and/or carriers.
wherein said composition is administered on a daily basis and said subject is in a non-agitated state.

52. A sublingual film composition for treating psychosis in a subject in need thereof, comprising:
i. a therapeutically effective amount of dexmedetomidine or a pharmaceutically acceptable salt thereof,
ii. one or more water-soluble polymers and
iii. one or more pharmaceutically acceptable excipients and/or carriers.
wherein said composition is administered on a daily basis and said subject is in a non-agitated state.

53. The film composition according to claim 51 or 52, wherein dexmedetomidine is present as dexmedetomidine hydrochloride.

54. The film composition according to claim 51 or 52, in the form of dosage unit, wherein amount of dexmedetomidine or a pharmaceutically acceptable salt thereof present per unit is about 0.5 μg to about 300 μg.

55. The film composition according to claim 54, wherein said dosage is about 2 μg to about 200 μg.

56. The film composition according to claim 51 or 52, wherein the film comprises dexmedetomidine or a pharmaceutically acceptable salt thereof together with one or more additional therapeutic agents.

57. The film composition according to claim 56, wherein said additional therapeutic agents are administered simultaneously, sequentially or separated by an appropriate period of time.

58. A method of stabilizing mood in a subject comprising administering dexmedetomidine or a pharmaceutically acceptable salt thereof in a range of about 10 μg to about 300 μg to the subject, optionally about 100 μg to about 300 μg to the subject,
wherein the subject has bipolar 1 disorder;
wherein the subject has mania, and
wherein the subject is not agitated.

59. The method of any of claims 51 to 58 wherein a first daily dose is administered in the morning and a second daily dose is administered in the evening.

60. The method of any of claim 59 wherein at least one dose is about 120 μg or about 180 μg.

61. A method of stabilizing mood in a subject comprising administering dexmedetomidine or a pharmaceutically acceptable salt thereof in a range of about 10 μg to about 300 μg to the subject, optionally about 100 μg to about 300 μg to the subject,
wherein the subject has bipolar 2 disorder;
wherein the subject has hypomania, and
wherein the subject is not agitated.

62. The method of any of claims 58 to 61 wherein a first daily dose is administered in the morning and a second daily dose is administered in the evening.

63. The method of claim 62 wherein at least one dose is about 120 μg or about 180 μg.
